(19) United States
(12) Patent Application Publication
(10) Pub. No.: US 2009/0281065 A1
(43) Pub. Date: Nov. 12, 2009

(54) Use of Lysophospholipids to Treat Inflammation

(75) Inventors: C. N. Ramchand, Chennai (IN); Bnedikt Sas, Stekene (BE); P. Kavitha, Chennai (IN)

Correspondence Address: DAVIS, BROWN, KOEHN, SHORS & ROBERTS, P.C.;THE DAVIS BROWN TOWER, 215 10TH STREET SUITE 1300, DES MOINES, IA, 50309, US

(73) Assignee: KEMIN INDUSTRIES, INC., Des Moines, IA (US)

(21) Appl. No.: 12/437,673

(22) Filed: May 8, 2009

Related U.S. Application Data
(60) Provisional application No. 61/051,703, filed on May 9, 2008.

(51) Int. Cl. A61K 31/66

(52) U.S. Cl. 514/114

(57) ABSTRACT

Phospholipids and lysophospholipids, more particular lysophospatidylcholine (LPC), known to be pro-inflammatory in certain amounts, are demonstrated to be useful in the prevention, the treatment or amelioration of the effects of inflammation.

This application claims priority to U.S. Patent Application Ser. No. 61/051,703, filed May 9, 2008, which is incorporated herein in its entirety by this reference.

### BACKGROUND OF THE INVENTION

The invention relates generally to methods of treatment or prevention of inflammation and, more specifically, to the administration of phospholipids and/ or lysophospholipids, more particularly lysophosphatidylcholine (LPC), to subjects to treat or prevent inflammation.

Diseases characterized by the presence of undesirable inflammation contribute significant loss of life expectancy and loss of well-being to a vast number of humans. These diseases include rheumatoid arthritis, psoriasis, asthma, colitis, multiple sclerosis, and systemic lupus erythematosus. Even diseases such as juvenile diabetes, atherosclerosis, and hypothyroidism have an inflammatory basis.

Treatment of inflammatory diseases in the past has been non-specific, based on broad-spectrum immunosuppressant drugs such as corticosteroids. Though beneficial, these drugs have universal side effects, which limit their use.

Recently, advances in science have identified ways to specifically block known targets in inflammation. The most successful of these approaches has been to block the molecule TNF alpha, a hormone-like molecule that signals information from one cell to other cells in the body. Such molecules are collectively known as cytokines. Cytokines like TNF play an essential role in normal immunity, but in some disease situations their levels are elevated and they exert deleterious effects on cell function and hence the well being of the individual. In disease situations such as colitis and rheumatoid arthritis, blockade of TNF has been associated with considerable benefit in patients.

Despite these successes, currently available TNF treatments are ineffective in approximately half of the patients and in most cases are administered with other drugs. They must be given by injection, and are often associated with adverse injection site reactions as well as inconvenience, and can be very costly, potentially limiting their availability to many patients.

Autoimmune disorders and asthma have drawn a great deal of attention from the pharmaceutical industry. The most successful new approach to treating inflammatory diseases in the last decade has addressed the pro-inflammatory role of cytokines, notably TNF-alpha, in these types of conditions. Three TNF-alpha blockers-Enbrel (etanercept), Remicade (infliximab), and Humira (adalimumab)-are now marketed for treating a range of autoimmune diseases, and they enjoy true blockbuster status, with aggregate sales topping $9 billion in 2006.

Nevertheless, the door remains open for improved therapeutics. Some patients do not respond to the TNF-alpha inhibitors; the effectiveness of the agents depends on long-term, even lifelong, administration; and they have been linked to tuberculosis, lymphoma, and other adverse effects.

Several new biological agents have begun to carve their own niches in the massive edifice of the TNF-alpha blockers. Orencia (abatacept), a T-cell co-stimulation modulator, was approved for the treatment of rheumatoid arthritis in 2005; Rituxan (rituximab), an anti-CD20 antibody, for rheumatoid arthritis in 2006; and Tysabri (natalizumab), an adhesion molecule blocker, for multiple sclerosis in 2006 and Crohn's disease in 2008.

A recurrent theme in discussions of treatment options for autoimmune diseases is the inadequacy of the standard of care. Management and pharmacotherapy are seeing only incremental improvements. Patients with asthma, an allergic disease, tend to fare well with the drugs currently available, though better treatment options with less potential for side effects are needed.

The complexity of the immune system provides both opportunity and challenge for the pharmaceutical industry. There is a seemingly endless list of cytokines, receptors, and enzymes that can be disrupted in patients with autoimmune and inflammatory diseases, and the sheer number of options leaves plenty of chances for companies-large established players and specialized newcomers alike-to carve out niches. On the other hand, the transition from brainstorm to marketed drug is fraught with pitfalls. Targeting a single receptor or protein often means being foiled by the immune system's redundancy, while cutting too wide a swath through the system can result in unexpected side effects.

Despite recent advances, there is still a requirement for new treatments for inflammatory diseases which are poorly controlled in some patients who do not respond to current medicines. New targets for treatment and potential new medicines need to be discovered and developed to help reduce this unmet need.

It is essential to test compounds in vivo because although the preliminary in vitro experiments will prove that a novel mechanism is involved in the process of inflammation/immunomodulation or that a novel compound can inhibit these processes, it does not reflect the complex interactions which take place in a whole animal, and is necessary in order to predict activity in humans.

Inflammatory response is an essential mechanism of defense against the attack of infectious agents, and it is also implicated in the pathogenesis of many acute and chronic diseases, including autoimmune disorders. Inflammation is a complex process, triggered by tissue injury that further evokes a response in terms of cell migration, release of chemical mediators, increase in the vascular permeability, extravasations of the blood fluids, all aimed to destroy and repair the injured tissue.

However, an inflammatory burst or an over-expression of the inflammatory response would be devastating. It is therefore necessary to curtail the amplification of inflammation with the use of anti-inflammatory drugs. Inflammation is commonly treated with the non-steroid anti-inflammatory drugs (NSAIDs). Although chemically diverse, basically all the NSAIDs exert anti-inflammatory effects by inhibiting the cyclooxygenase activity, thereby suppressing the production of the pro-inflammatory prostaglandins. It is to be noted that most of the potent and classical NSAIDs that are administered to alleviate inflammatory conditions, are non-specific and along with their inhibition towards the cyclooxygenase 2 (COX-2) enzyme, they also impair COX-1, which leads to gastrointestinal toxic side effects. COX2 is usually absent or present in only small amount in the cells under basal levels. However, it is induced in cells involved in inflammatory stimuli or cytokines.

Typical inflammatory based diseases are asthma, arthritis, psoriasis and Inflammatory Bowel Diseases.

Classic asthma definitions focus on the disease's impact on lung function, including airflow limitation, its reversibility and airway hyperresponsiveness. However, the Global Initiative for Asthma (GINA) proposed a more practical definition that appreciates the underlying role of inflammation: “Asthma is a chronic inflammatory disorder of the airways in which many cells and cellular elements play a role. The chronic inflammation causes an associated increase in airway hyperresponsiveness that leads to recurrent episodes of wheezing, breathlessness, chest tightness, and coughing, particularly at night or in the early morning. These episodes are usually associated with widespread but variable airflow obstruction that is often reversible either spontaneously or with treatment.” (GINA Global Strategy for Asthma Management and Prevention, 2005) Asthma is an epidemic that currently affects approximately 7% of the general population in the seven major markets. The disease is estimated to affect as many as 300 million people worldwide, a number that could increase by a further 100 million by 2025, according to the Global Burden of Asthma (Beasley et al., 2004).

Rheumatoid arthritis (RA) is an inflammatory, autoimmune disease that affects the lining of the joints, causing pain, swelling, and reduced mobility for the patient. The prevalence of RA ranges from 0.24% to 1.0% among the populations of the seven major markets (the US, Japan, France, Germany, Italy, Spain and the UK. A geographical/ethnic trend seems to be present in Europe, where RA is more common in the Anglo-Saxon population than in the Mediterranean population. In addition, RA cases seem to be less severe (Drosos et al., 1992) in southern Europe; however, epidemiological studies of RA in that region are rare. The available data suggest that RA in southern Europe presents a particular clinical profile, in that it is less prevalent, and milder, with fewer extra-articular and radiological manifestations. Environmental and lifestyle factors may contribute to this different profile. Dietary factors such as olive oil and fish consumption could offer a protective effect against disease development and severity (Alamanos et al., 2005). The RA market is growing rapidly in terms of sales, with an estimated 19% CAGR for 2003-06. The majority of this growth is driven by the US market.

The key unmet need of the RA market can generally be considered the discovery of a cure. However, while basic research into the origin and exact mechanisms of the disease provide increasingly effective medication, a cure remains elusive.

Therefore, the unmet needs of the current market do focus on the key clinical factors of improving the safety and efficacy of the available treatments. It is impossible to deny that improvement in these factors has moved forward significantly in the last decade, however, a number of new barriers to using this technology exist in both clinical and environmental areas.

Psoriasis is a chronic, recurring disease that causes one or more raised, red patches that have silvery scales and a distinct border between the patch and normal skin. The patches of psoriasis occur because of an abnormally high rate of growth of skin cells. The reason for the rapid cell growth is unknown, but a problem with the immune system is thought to play a role. Psoriasis is a relatively common skin condition, affecting 1-3% of the world's population and can be hereditary. It is rarely life threatening, but it often results in severe morbidity and disability. Psoriasis prevalence is more common in the Caucasian population and is low in Japan and East Asia in general and is extremely low to non-existent in ethnic groups native to North and South America. There is currently no cure for psoriasis. Psoriasis can have a particularly negative effect on quality of life, affecting a sufferer's physical, social, and psychological functioning so the need for effective therapies is high. Prevalence of psoriasis is the highest of the autoimmune diseases, but this is deceptive in terms of market size, as the severe patient population is comparatively small.

In 2005 Datamonitor estimated the psoriasis market to have sales of $1.95 billion, up 41% from a 2004 value of $1.39 billion across the seven major markets. This increase is driven by the use of biologic therapies, exemplified by the fact that the market volume increased by a much slower rate of 4%, a trend that is forecast to decline in the future.

Crohn's disease and ulcerative colitis occur due to inappropriate and/or excessive responses to antigens present in the normal bacterial micro flora (Stefan Fichtner-Feigl et al, 2005). Crohn's disease is characterized by a transmural, granulomatous inflammation occurring anywhere in the alimentary canal but is usually centered in the terminal ileum and ascending colon; ulcerative colitis, in contrast, is marked by a superficial inflammation causing epithelial cell destruction (ulceration) that is centered in the rectum and colon (Podolsky, 2002 and Bouma & Strober, 2003). Despite having a common basis in over responsiveness to mucosal antigens, the 2 diseases have considerably different pathophysiologies. Crohn's disease is associated with a Th1 T cell-mediated response induced by IL-12 and possibly IL-23, whereas ulcerative colitis is associated with an atypical Th2-mediated response characterized by NKT cell secretion of IL-13 (Uhlig et al, 2003; Monteleone, 1997; Mannon, 2004; Fuss, 2004 and Schmidt, 2005).

The worldwide incidence of IBD is estimated at approximately 1/1000 persons (Podolsky, 2002), with incidence rates for ulcerative colitis (UC) and Crohn's disease (CD) in European countries, calculated at 10.4/100 000 and 5.6/100 000, respectively (Binder, 2004). Individuals suffering from other autoimmune diseases (Miller et al., 2003) are more likely to experience IBD, as arguably are Caucasians in comparison to ethnic African (Mwantembe et al., 2001). The economic cost of annual health care in Europe is estimated at [symbol] 1524 and [symbol] 2548 per patient for UC and CD respectively (Odes et al., 2006), and in excess of $55,000 per patient in the US (Cohen and Roth, 2000). IBD epitomizes one of the pathological disorders posing the most compelling need for new therapeutic strategies. Pharmacological therapies, including glucocorticoids, immunosuppressants and aminosalicylates have proven difficult and complex (Sands, 2000). The efficacy of these drugs in managing inflammatory conditions has been overshadowed by substantial side effects, thus limiting the continuation of treatment. Furthermore, some patients are refractory even to the combined use of these agents (Lichtenstein, 2001; Baidoo & Lichtenstein, 2005), and surgical resection of the colon and ileostomy is frequently required (Lapidus et al., 1998).

The precise etiology of the disease remains an enigma, but is likely to involve multifactor interactions among genetic factors, immunological factors and environmental triggers (Sandborn & Targan, 2002). Chromosome 16 in particular is a well-replicated linkage of CD and contains the CARD15 susceptibility gene that is involved in the activation of the pro-inflammatory transcription factor, NF-Kb (Ogura et al., 2001), via the interaction with MAP-Kinase and ERK1 (Hugot et al., 2001). CARD 15, contains three rare disease susceptibility alleles (Hugot et al., 2001), and at least one of these alleles is present in 30-40 percent of European patients (Lesage et al., 2002), compared with 14 percent of controls.

The most notable factor in the etiology of the disease however, is the disregulation of mucosal inflammatory response to bacterial flora (Shanahan, 2002). Studies on patients and animal models of colitis suggest that an initial immunological challenge involving an abnormal host response to endogenous or environmental antigens or microbes causes tissue injury (Bamias et al., 2005; Hendrickson, 2002). The mucosal immune system normally controls the down regulation of the enhanced host defense system, thereby preventing damage to the tissue. In IBD, however, the loss or deterioration of the immunomodulating mechanisms provides the opportunity for the normally down-regulated responses to amplify uncontrollably into a chronic, inflammatory condition (Strober et al., 2002).

Lysophosphatidylcholine is acting on a very different and early step in the inflammation pathway compared to the COX2 inhibitors or NSAIDS. We have found that LPC at the right dose will act on the phosporlipase A2 via a negative feed back mechanism, driving the inflammation back. LPC can be obtained from nature, e.g., via enzymatic hydrolyses of lecithin into lysolecithin (U.S. Pat. No. 6,068,997). A typical content of LPC is >3% of the total phospholipids. In addition, LPC can also be obtained in a pure form via chemical synthesis. The synthetic pathway is very well known and various approaches exist.

Rat model of TNBS colitis, commonly used model for human IBD, is characterized by exaggerated Th1 cell response associated with up regulation of proinflammatory cytokines and down regulation of regulatory cytokines. The current study was undertaken to highlight the functions of the primary inflammatory pathway, phospholipase A₂ (PLA₂), in the production of eicosanoid and cytokines, and their regulation and significance to IBD. It also further cited the potential inhibitory pathways and emphasized the grounds that render the inhibition of the PLA₂ pathway particularly important in the treatment of this disease.

There have been several reports on the immunopotentiating activity of LPC, which is one of the key ingredients of Lysoforte™. LPC is a pro-inflammatory and atherogenic serum lysophospholipid, which activates a variety of cell types, including the vascular endothelium (Kohno et al., 1998; Kume et al., 1992). LPC is known to exert its effects by binding to G2A and/or GPR4 receptors present on the cell surface. LPC has been found to be associated with a wide range of activities such as induction of growth factor expression by endothelial cells and monocytes; the suppression of endothelium dependent vasorelaxation, chemoattraction of monocytes, stimulation of smooth muscle cell proliferation and activation of protein kinase C in intact vascular segments leading to increased superoxide production (McIntyre et al., 1999; Wu et al., 1998).

LPC could enhance antibody formation and act as an antigen for antibodies binding to oxidized low density lipoproteins (LDL) (Huang, 2002). The antigenicity of LPC is suggested to depend on its special structure with an empty sn-2 position. It has also been proposed that LPC itself is not likely to act as an antigen, instead it forms immunogenic complexes with peptides, which may induce and potentiate immune reactions (Wu et al., 1998). C-reactive protein, which is elevated in the blood in inflammatory conditions, is known to bind to LPC in membranes of injured cells, thus forming a potential candidate for an LPC complexing protein (Hack et al., 1997). Elevated activity of phospholipase A2, which produces LPC, has been observed in a variety of immunological/inflammatory diseases (Pruzanski et al., 1994). However, it is important to highlight that all the immunostimulatory effects of LPC is observed at a concentration range between 10 and 50 mM in serum (McIntyre et al., 1999).

### SUMMARY OF THE INVENTION

The invention consists of the use of lysophospholipids, more particular lysophosphatidylcholine (LPC), to treat or prevent inflammation in animal subjects, including humans. The effect of lysophospholipids, more particular lysophosphatidylcholine (LPC), on inflammation has been demonstrated in chickens, rats, and humans. Administration of effective amounts of LPC was specifically demonstrated to improve feed conversion rate and weight gain in chickens, ameliorate the effects of TNBS-induced colitis in rats, and ameliorate the symptoms of arthritis and asthma in humans.

Another embodiment of the invention combines one or more lysophospholipids with one or more pholpholipids, the combination of which is administered in a therapeutically effective amount to treat or prevent the effects of inflammation.

Yet another embodiment of the invention combines one or more lysophospholipids with either or all of omega-3 fatty acids, prebiotics, probiotics, and lutein, the combination of which is administered in a therapeutically effective amount to treat or prevent the effects of inflammation.

Still another embodiment of the invention combines one or more known anti-inflammatory compounds with compounds of the present invention, the combination of which is administered in a therapeutically effective amount to treat or prevent the effects of inflammation.

The invention also includes formulations of one or more phospholipids and/or one or more lysophospholipids, either alone or in combination with known anti-inflammatory compounds, omega-3 fatty acids, prebiotics, probiotics, and lutein.

### BRIEF DESCRIPTION OF THE FIGURES

FIG. 1 is a chart of the increase in toe web thickness in response to treatment with PHA-P.

FIG. 2 is a chart of feed conversion rate consolidated over 6 weeks, in groups.

FIG. 3 is a chart of consolidated weight gain in all the groups over 45 days.

FIG. 4 is a chart of feed consumption in all the groups over six weeks.

FIG. 5 is a chart of the difference in the ESR % pre and post trial.

FIG. 6 is a chart of the % difference in the CRP pre and post trial.

FIG. 7 is a chart of the body weight changes of rats induced with TNBS colitis; the data are expressed as means ±SEM (n=6 in each group).

FIG. 8 is a chart of the results of a cytotoxicity test of different sources of LPC; the concentrations listed in the box are the concentration of LPC contained in each sample.

FIG. 9 is a chart of the results of an inflammation test without TNF-α; the inflammation activity was represented by luciferase activity measured in HepG₂-NFκB stable cells; all the concentrations listed are LPC content; the asterisk means statistical difference (p<0.01).

FIG. 10 is a chart of the results of an inflammation test with TNF-α; the inflammation activity was represented by luciferase activity measured in HepG₂-NFκB stable cells; all the concentrations listed are LPC content; the asterisk means statistical difference (p<0.01).

### DETAILED DESCRIPTION OF PREFERRED EMBODIMENTS

### Definitions

As used herein, the term “inflammatory disease” refers to either an acute or chronic inflammatory condition, which can result from infections or non-infectious causes. Various infectious causes include meningitis, encephalitis, uveitis, colitis, tuberculosis, dermatitis, and adult respiratory distress syndrome. Non-infectious causes include trauma (burns, cuts, contusions, crush injuries), autoimmune diseases, and organ rejection episodes. Thus, in specific embodiments, an inflammatory condition results from a condition selected from the group that includes: atherosclerosis (arteriosclerosis); autoimmune conditions, such as multiple sclerosis, systemic lupus erythematosus, polymyalgia rheumatica (PMR), gouty arthritis, degenerative arthritis, tendonitis, bursitis, psoriasis, fibrosis, arthrosteitis, rheumatoid arthritis and other forms of inflammatory arthritis, Sjogren's Syndrome, progressive systemic sclerosis (scleroderma), ankylosing spondylitis, polymyositis, dermatomyositis, pemphigus, pemphigoid, Type I diabetes mellitus, myasthenia gravis, Hashimoto's thyroditis, Graves' disease, Goodpasture's disease, mixed connective tissue disease, sclerosing cholangitis, inflammatory bowel disease including Crohn's Disease (regional enteritis) and ulcerative colitis, pernicious anemia, inflammatory dermatoses; usual interstitial pneumonitis (UIP), asbestosis, silicosis, bronchiectasis, berylliosis, talcosis, all forms of pneumoconiosis, sarcoidosis (in the lung and in any other organ), desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa); inflammatory dermatoses not presumed to be autoimmune; chronic active hepatitis; delayed-type hypersensitivity reactions (e.g., poison ivy dermatitis); pneumonia or other respiratory tract inflammation due to any cause; Adult Respiratory Distress Syndrome (ARDS) from any etiology; encephalitis with inflammatory edema; immediate hypersensitivity reactions including, but not limited to, asthma, hayfever, cutaneous allergies, acute anaphylaxis; diseases involving acute deposition of immune complexes, including, but not limited to, rheumatic fever, acute and/or chronic glomerulonephritis due to any etiology, including specifically post-infectious (e.g., post-Streptococcal) glomerulonephritis, acute exacerbations of Systemic Lupus Erythematosus; pyelonephritis; cellulitis; cystitis; acute and/or chronic cholecystitis; and conditions producing transient ischemia anywhere along the gastrointestinal tract, bladder, heart, or other organ, especially those prone to rupture; sequelae of organ transplantation or tissue allograft, including allograft rejection in the acute time period following allogeneic organ or tissue transplantation and chronic host-versus-graft rejection. The term “inflammatory disease” also includes appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, tonsillitis, uveitis, vaginitis, vasculitis, vulvitis, and vulvovaginitis, angitis, chronic bronchitis, osteomylitis, optic neuritis, temporal arteritis, transverse myelitis, necrotizing fascilitis, hepatitis, and necrotizing enterocolitis.

Any anti-inflammatory compound(s), or a pharmaceutically acceptable salt thereof, may be used in combination with the compound(s) of the present invention for the treatment of an inflammatory disease, or for the treatment of inflammation and pain, according to the present invention. Suitable anti-inflammatory compounds include, but are not limited to: non-steroidal anti-inflammatory drugs (NSAIDs) (e.g., aspirin, ibuprofen, naproxen, methyl salicylate, diflunisal, indomethacin, sulindac, diclofenac, ketoprofen, ketorolac, carprofen, fenoprofen, mefenamic acid, piroxicam, meloxicam, celecoxib, valdecoxib, parecoxib, etoricoxib, and nimesulide), corticosteroids (e.g., prednisone, betamethasone, budesonide, cortisone, dexamethasone, hydrocortisone, methylprednisolone, prednisolone, tramcinolone, and fluticasone), anti-malarial agents (e.g., hydroxychloroquine), acetaminophen, glucocorticoids, steroids, beta-agonists, anticholinergic agents, methyl xanthines, gold injections, sulphasalazine, penicillamine, anti-angiogenic agents, dapsone, psoralens, anti-viral agents, and antibiotics.

As used herein, the term “therapeutically effective amount” refers to the amount/dose of a compound or pharmaceutical composition that is sufficient to produce an effective response (i.e., a biological or medical response of a tissue, system, animal or human sought by a researcher, veterinarian, medical doctor or other clinician) upon administration to a subject. The “therapeutically effective amount” will vary depending on inter alia the disease and its severity, and the age, weight, physical condition and responsiveness of the subject to be treated.

As used herein, the term “treating” refers to preventing or delaying the appearance of clinical symptoms of a disease or condition in a subject that may be afflicted with or predisposed to the disease or condition, but does not yet experience or display clinical or subclinical symptoms of the disease or condition. “Treating” also refers to inhibiting the disease or condition, i.e., arresting or reducing its development or at least one clinical or subclinical symptom thereof. “Treating” further refers to relieving the disease or condition, i.e., causing regression of the disease or condition or at least one of its clinical or subclinical symptoms. The benefit to a subject to be treated is either statistically significant or at least perceptible to the subject and/or the physician.

As used herein, the term “pharmaceutically acceptable” refers to molecular entities and compositions that are “generally regarded as safe”—e.g., that are physiologically tolerable and do not typically produce an allergic or similar untoward reaction, such as gastric upset and the like, when administered to a human. In another embodiment, this term refers to molecular entities and compositions approved by a regulatory agency of the federal or a state government or listed in the U.S. Pharmacopeia or another generally recognized pharmacopeia for use in animals, and more particularly in humans.

Phospholipids are a class of lipids and are a major component of all cell membranes. Most phospholipids contain a diglyceride, a phosphate group, and a simple organic molecule such as choline. Lysophospholipids are derivatives of phospholipids that lack one of its fatty acyl chains due to its hydrolytic removal. The names and structures of the principal lysophospholipids are set out below:

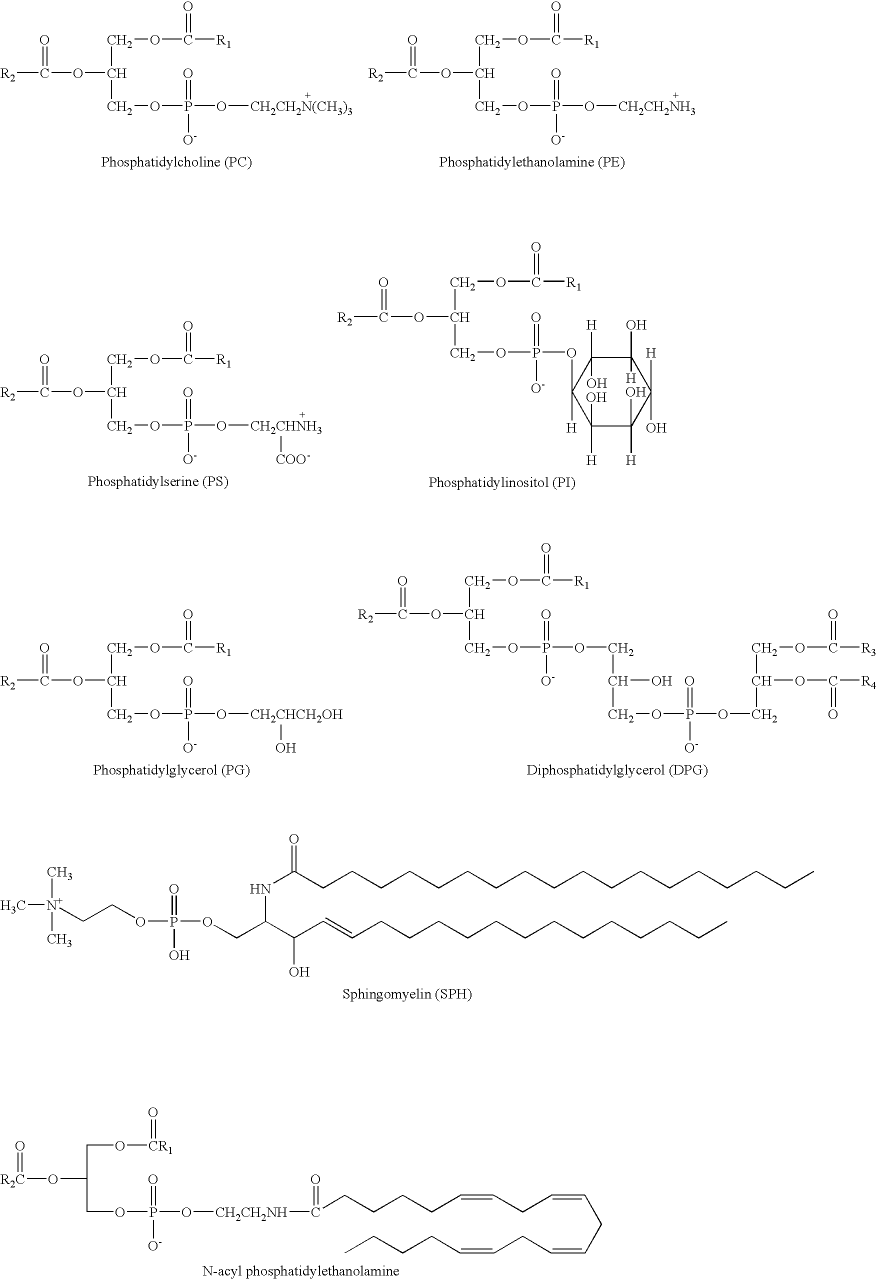

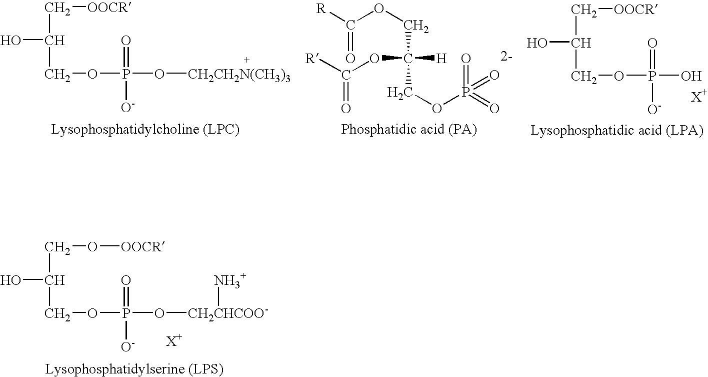

One commercially available source of lysophospholipids is Lysoprin™. The composition of Lysoprin™ is provided in the following table:

| Abbreviation | Lysophospholipids | Wt % |
|---|---|---|
| PC | Phosphatidylcholine | 13.02% |
| LPC - 1 | Lysophosphatidylcholine | * |
| LPC - 2 | Lysophosphatidylcholine | 2.29 |
| PI | Phosphatidylinositol | 8.14 |
| LPI | Lysophosphatidylinositol | 1.08 |
| PS Na | Phosphatidylserine Sodium | 0.40 |
| LPS | Lysophosphatidylserine | * |
| SPH | Sphingomyelin | * |
| PE | Phosphatidylethanolamine | 9.74 |
| LPE | Lysophosphatidylethanolamine | 1.05 |
| APE | N-acylphosphatidylethanolamine | 1.29 |
| PG | Phosphatidylglycerol | 0.64 |
| DPG | Diphosphatidylglycerol | 0.63 |
| PA | Phosphatidic acid | 3.15 |
| LPA | Lysophosphatidic acid | 0.42 |
| other | | 1.42 |
| | | 42.50 |

* below the detection limit

### Formulations

The invention also provides the use as above wherein the medicament is for administration as a unit dose. In another embodiment of the invention, the unit dose is containing the active ingredient in an amount from about 10 μg/kg to 10 mg/kg body weight, in another embodiment from about 25 μg/day/kg to 1.0 mg/day/kg, in yet another embodiment from about 0.1 mg/day/kg to 1.0 mg/day/kg body weight. In another embodiment, the unit dose is containing the active ingredient in an amount from 0.1 mg/day/kg to 1.0 mg/day/kg body weight.

According to the invention, the compounds mentioned above may be used as the base of the compound or as a pharmaceutically acceptable acid addition salt thereof or as an anhydrate or hydrate of such salt. According to the invention, the compounds mentioned above or a pharmaceutically acceptable salt thereof may be administered in any suitable way e.g. orally or parenterally, and it may be presented in any suitable form for such administration, e.g. in the form of tablets, capsules, powders, syrups or solutions or dispersions for injection. In another embodiment, and in accordance with the purpose of the present invention, the compound of the invention is administered in the form of a solid pharmaceutical entity, suitably as a tablet or a capsule or in the form of a suspension, solution or dispersion for injection. The compound of the invention is most conveniently administered orally in unit dosage forms such as tablets or capsules, containing the active ingredient in an amount from about 10 μg/kg to 10 mg/kg body weight, for example 25 μg/day/kg to 1.0 mg/day/kg.

Compounds of the present invention may be administered as an oral dose form, such as a solid oral dose form, typically tablets or capsules, or as a liquid oral dose form, or may be administered in an immediate release dosage form or a controlled or sustained release dosage form. The compounds may be conveniently administered orally in unit dosage forms, such as tablets or capsules, containing the active ingredient in an amount from about 0.1 to about 150 mg/day, from about 0.2 to about 100 mg/day, from about 0.5 to about 50 mg/day, from about 0.1 to about 50 mg/day, from about 1 to about 15 mg/day, or from about 2 to about 5 mg/day. Typically, the pharmaceutical composition comprises from about 0.5 mg to about 20 mg, such as about 0.5 mg, about 1 mg, about 1.5 mg, about 2 mg, about 2.5 mg, about 3 mg, about 3.5 mg, about 4 mg, about 4.5 mg, about 5 mg, about 5.5 mg, about 6 mg, about 6.5 mg, about 7 mg, about 7.5 mg, about 8 mg, about 8.5 mg, about 9 mg, about 9.5 mg, about 10 mg, about 10.5 mg, about 11 mg, about 11.5 mg, about 12 mg, about 12.5 mg, about 13 mg, about 13.5 mg, about 14 mg, about 14.5 mg, about 15 mg, about 15.5 mg, about 16 mg, about 16.5 mg, about 17 mg, about 17.5 mg, about 18 mg, about 18.5 mg, about 19 mg, about 19.5 mg or about 20 mg of one or more of the compounds.

In one embodiment, the compound(s) of the present invention are administered once daily (for example, in the morning or afternoon) using doses of about 2.5 mg to about 20 mg. In another embodiment, the compound(s) are administered in a more prolonged and continuous release, e.g., administration 2-3 times daily with low doses or a modified release formulation prepared using conventional methods known in the art, such that about 5 to about 50 mg administered to the subject per 24 hour period.

According to the present invention, the compound(s) of the present invention or a pharmaceutically acceptable salt thereof may be administered in any suitable way, e.g., orally or parenterally, and it may be presented in any suitable form for such administration, e.g., in the form of tablets, capsules, powders, syrups or solutions or dispersions for injection, or as an inhalant. In another embodiment, and in accordance with the purpose of the present invention, the compound(s) of the present invention are administered in the form of a solid pharmaceutical entity, suitably as a tablet or a capsule or in the form of a suspension, solution or dispersion for injection. Additionally, the compound(s) of the present invention may be administered with a pharmaceutically acceptable carrier, such as an adjuvant and/or diluent.

Methods for the preparation of solid or liquid pharmaceutical preparations are well known in the art. See, e.g., Remington: The Science and Practice of Pharmacy, 21st ed., Lippincott Williams & Wilkins (2005). Tablets may thus be prepared by mixing the active ingredients with an ordinary carrier, such as an adjuvant and/or diluent, and subsequently compressing the mixture in a tabletting machine. Non-limiting examples of adjuvants and/or diluents include: corn starch, lactose, talcum, magnesium stearate, gelatine, lactose, gums, and the like. Any other adjuvant or additive such as colorings, aroma, and preservatives may also be used provided that they are compatible with the active ingredients. The pharmaceutical compositions of the invention thus typically comprise an effective amount of the compound(s) of the present invention and a pharmaceutically acceptable carrier.

The compounds may be administered systemically in a form selected from the group consisting of: an aerosol suspension of respirable particles; a liquid or liquid suspension for administration as nose drops or nasal spray; a nebulized liquid for administration to oral or nasopharyngeal airways; an oral form; an injectable form; a suppository form; and a transdermal patch or a transdermal pad; such that a therapeutically effective amount of said compound contacts the sites of allergic reactions and inflammation of said subject via systemic absorption and circulation.

One such means involve an aerosol mixture of respirable particles comprised of the active compounds, which the subject inhales. The therapeutic compound is absorbed into the bloodstream via the lungs in a pharmaceutically effective amount. The respirable particles may be liquid or solid, with a particle size sufficiently small to pass through the mouth and larynx upon inhalation; in general, particles ranging from about 1 to 10 microns, but more preferably 1-5 microns, in size are considered respirable.

Another means of delivering the therapeutic compound to sites of allergic reactions and inflammation involve administering a liquid/liquid suspension in the form of nasal drops of a liquid formulation, or a nasal spray of respirable particles which the subject inhales. Liquid pharmaceutical compositions of the active compound for producing a nasal spray or nasal drops are prepared by combining the active compounds with a suitable vehicle, such as sterile pyrogen free water or sterile saline by techniques known to those skilled in the art.

### EXAMPLE 1

### Effect of Lysophospholipids on Inflammation in Broilers

The immunostimulating activity of LPC (in the form of Lysoforte™ brand lysophospholipids from Kemin Industries, Inc.) was evaluated by assessing the cell mediated and humoral immune responses in broilers fed with Lysoforte™ and the efficacy was compared with two other sources of lysophospholipids. Cutaneous delayed type hypersensitivity (DTH) response elicited in chickens by an intra dermal injection of phytohemagglutinin-P (PHA-P) was used to assess the in vivo cell mediated immune response. The results obtained indicated that Lysoforte™ fed group has significantly improved (p<0.05) inflammatory response to PHA-P injection compared to the control (untreated) and groups treated with competitor products. Birds fed with competitor products having lysophospholipids did not show any enhancement in the cell-mediated immune response. The humoral immune response in birds was measured from the antibody titers produced against sheep RBC (SRBC). The current study shows that Lysoforte™ has a positive effect on cell-meditated immune response in broilers, which is an added benefit of Lysoforte™ supplementation to the already established advantages such as weight gain and feed efficiency in broilers.

### Materials and Methods

### Materials

Samples of LPC, other lysophospholipids and lecithins for the trial were Lysoforte™; Lysoprin™;(Lovesgrove Research) and Bolec™ (Loders Crokaan) brands.

Reagents and equipments: 1. Phosphate buffered saline (PBS); (pH-7.4) 2. 0.01% EDTA (Himedia, India); 3. Sterile syringes (2 ml); 4. ‘V’ bottom 96 well assay plates (Tarson); 5. Centrifuge tubes (Tarson); 6. Phytohemagglutinin-P (PHA-P) (GeNei, India); 7. Digital Micrometer (Multiyo, Japan); and 8. 0.5% and 0.75% sheep RBC suspension in PBS.

### Methods

The experiment was conducted on straight run commercial hybrid broiler chicks (Vencobb 400) for a period of six weeks starting on the first day of hatching. Each group consisted of 8 birds. Table 1 shows the treatment given to each group of birds during the trial period.

TABLE 1
Experimental groups

| Group | Treatment |
|---|---|
| Control | Commercial broiler feed (PS: 15 d, S: 10 d, F: 17 d). |
| Treatment 1 (T1) | Control feed + Lysoforte ™ (500 g/T) |
| Treatment 2 (T2) | Control feed + Competitor brand A |
| | (500 g/T; as per supplier instructions) |
| Treatment 3 (T3) | Control feed + Competitor brand B |
| | (600 g/T; as per supplier instructions) |

Note:
PS: Prestarter;
S: Starter;
F: Finisher

### Antibody Response to Sheep RBC:

The humoral immune response in birds was measured from the antibody titers produced against sheep RBC (Wegmann and Simthies, 1966). About 10 ml of blood was collected from jugular vein of an adult sheep into sterile tubes containing 1 ml EDTA (0.01%) as anticoagulant. Red blood cells were harvested by repeated centrifugation (3000 rpm/10 min) and washing (4 times) with phosphate buffered saline (pH-7.4). Finally, the RBC was diluted with PBS to get 0.1% RBC suspension. 0.5 ml of 0.1% SRBC suspension (in PBS) was injected into wing vein of birds on 28th and 35th day of the experiment and the subsequent immune response was quantified by collection of blood on the 7th day post inoculation (PI). Antibody titres against SRBC injection were measured using 0.75% SRBC suspension (in PBS) by Haemagglutination test procedure.

### Haemagglutination Test

This test was performed in ‘V’ bottom 96 well assay plates. Serum was separated from the collected blood samples. Thirty μl serum samples from each bird were added separately to the first well in each row of the plate and were serially diluted with 30 μl of PBS (pH 7.4) in the subsequent wells up to the 10th well in each row. Last two wells in each row were kept as controls without serum sample. Thirty pt of 0.75% SRBC suspension (in PBS) was added into each well and the plates were incubated at room temperature for 60 min. After incubation, the agglutination results were read and the antibody titer was expressed as the reciprocal of the highest dilution that gave agglutination of SRBC.

### Cutaneous Delayed Type Hypersensitivity (DTH) Response to PHA-P

Cutaneous DTH response in broilers was performed as per the method of Corrier and De Loach (1990). The pre-injection thickness of the right and left toe web of the birds was measured using a digital micrometer on 23rd day of the trial. After the measurement, 0.5 ml of PHA-P (50 mg) was injected into right toe web, whereas the left toe web was used as a control (injected with 0.5 ml PBS). Thickness of the toe web was measured 24 h post-injection using a digital micrometer. The increase in thickness of the right toe web compared to that of the left toe web was used as an index of cell-mediated immune response. The same experiment was repeated on 41st day of the trial.

### Results and Discussion

### Effect of Lysophospholipids (Lysoforte™) on the in Vivo Cell Mediated Immune Response

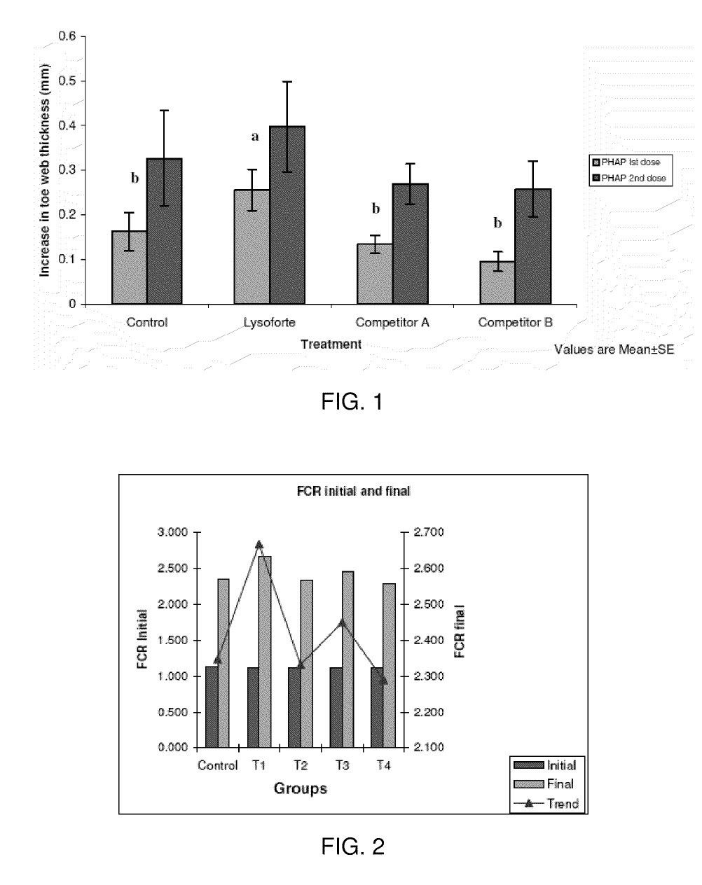

FIG. 1 shows the mean increase in the toe web thickness for each treatment group 24 h after PHA-P injection. It was observed that the Lysoforte™ fed group of birds had improved (p<0.05) inflammatory response to PHA-P injection compared to the control and competitor products. Birds treated with competitor products did not show any enhancement in the cell-mediated immune response, in fact there was a reduced inflammatory response compared to the control although not significant. The results of second experiment with PHA-P performed on 41st day of trial also showed a similar trend with the Lysoforte™ fed group giving a better immune response (although not significant) than the competitor groups.

The amount of LPC (48 mM) fed to the birds through the existing dose of Lysoforte™ falls within the range (10-50 mM) that is known to exert immunostimulatory activity (McIntyre et al., 1999). On the contrary, LPC at concentrations above 50 mM in serum have been shown to be cytotoxic (Frostegard et al., 1997).

### Effect of Lysophospholipids (Lysoforte™) on Humoral Immune Response

Table 2 and Table 3 show the antibody response to the administration of primary and booster doses of SRBC, respectively.

TABLE 2
Antibody titer levels against SRBC (Primary dose)

| Samples | Control | T1 | T2 | T3 |
|---|---|---|---|---|
| 1 | 16 | 64 | 128 | 8 |
| 2 | 64 | 512 | 128 | ND |
| 3 | ND | 64 | 128 | 256 |
| 4 | 128 | 32 | ND | 512 |
| 5 | 16 | 16 | 32 | 32 |
| 6 | 32 | 32 | 8 | 8 |
| 7 | 32 | 64 | 8 | 1024 |
| 8 | 128 | 64 | 8 | 64 |

TABLE 3
Antibody titer levels against SRBC (Booster dose)

| Samples | Control | T1 | T2 | T3 |
|---|---|---|---|---|
| 1 | 256 | 256 | 128 | 8 |
| 2 | 2048 | 1024 | 1024 | ND |
| 3 | 512 | 512 | 1024 | 1024 |
| 4 | ND | 4 | ND | ND |
| 5 | 128 | 8 | 1024 | 128 |
| 6 | 4096 | 1024 | ND | 1024 |
| 7 | 512 | 1024 | ND | 1024 |
| 8 | 4096 | 1024 | 1024 | 1024 |

ND: Not detected

### CONCLUSION

Lysophospholipids (Lysoforte™) stimulate the cell-mediated immunity of broiler birds as observed by the enhanced inflammatory response to PHA-P administration. This is an added benefit of feeding the broilers with this product in addition to the proven advantages such as weight gain and feed efficiency.

### REFERENCES

1. Corrier, D. E. and DeLoach, J. R. (1990) Evaluation of cell-mediated cutaneous basophil hypersensitivity in young chickens by an interdigital skin test. Poult. Sci., 69(3): 403-408.
2. Frostegard, J., Huang, Y. H., Ronnelid, J. and Elinder, L. S. (1997) Platelet-Activating factor and oxidized LDL induce immune activation by a common mechanism. Arterioscler. Thromb. Vasc. Biol., 17: 963-968.
3. Hack, C. E., Wolbink, G. J., Schalkwijk, C., Speijer, H., Hermens, W. T. and van den Bosch, H. (1997) A role for secretory phospholipase A2 and C-reactive protein in the removal of injured cells. Immunol Today.,18:111-115.
4. Huang, M. C., Graeler, M., Shankar, G., Spencer, J. and Goetzl, E. J. (2002) Lysophospholipid mediators of immunity and neoplasia. Biochim Biophys Acta, 1582 (1-3): 161-167.
5. Kohno, M., Yokokawa, K., Yasunari., K., Minami, M., Kano, H., Hanehira, T. and Yoshikawa, J. (1998). Induction by lysophosphatidylcholine, a major phospholipid component of atherogenic lipoproteins, of human coronary artery smooth muscle cell migration. Circ., 98: 353-359.
6. Kume, N., Cybulsky, M. I. and Gimbrone, Jr, M. A. (1992) Lysophosphatidylcholine, a component of atherogenic lipoproteins, induces mononuclear leukocyte adhesion molecules in cultured human and rabbit arterial endothelial cells. J. Clin. Invest., 90: 1138-1144.
7. McIntyre, T. M., Zimmerman, G. A. and Prescott, S. M. (1999) Biologically active oxidized phospholipids. J. Biol. Chem., 274(36); 25189-25192.
8. Pruzanski, W., Goulding, N. J., Flower, R. J., Gladman, D. D., Urowitz, M. B., Goodman, P. J., Scott, K. F. and Vadas, P. (1994) Circulating group II phospholipase A2 activity and antilipocortin antibodies in systemic lupus erythematosus; correlative study with disease activity. J Rheumatol., 21: 252-257.
9. Vandaveer, S. S., Erf, G. F. and Durdik, J. M. (2001). Avian T helper one/two immune response balance can be shifted toward inflammation by antigen delivery to scavenger receptors. Poult. Sci., 80:172-181.
10. Wegmann, T. G. and Smithies, O. (1966) A simple hemagglutination system requiring small amounts of cells and antibodies. Transfusion, 6:67-73.
11. Wu, R., Huang, Y. H., Elinder, L. S. and Frostegard, J. (1998) Lysophosphatidylcholine is involved in the antigenicity of oxidized LDL. Arterioscler. Thromb. Vasc. Biol., 18:626-630.

### EXAMPLE 2

### Effect of Lysophospolipids in Immune Potentiation in Broilers

An experiment was conducted in broilers to evaluate the efficacy of lysophosphatidylcholine (LPC) as Lysoforte™ booster, lutein and chromium propionate, individually and in a combination, towards immune potentiation in poultry. The study was carried out in Cobb 400 broiler chickens and the specific antibody response to New Castle disease virus (NDV) and Infectious Bursal Disease Virus (IBD) was quantified. The relative weights of the immune organs were measured twice during the study period of 45 days and the growth parameters like feed intake, weight gain and FCR were recorded. From the antibody titer values and immune organ weights obtained, it was observed that the LPC treated group showed a tendency towards enhanced immune response. It was also found that the combination group showed a lower immune response in terms of the antibody titers and the immune organ weights. There was a significant reduction in the weight gain and the feed intake in the LPC treated group. The FCR was higher in the LPC treated group. This is indicative of a higher metabolic rate in the LPC treated group. Further studies need to be conducted elaborately to evaluate the dose response with LPC treatment.

Lysophosphatidylcholine (LPC), 1-acyl-sn-glycero-3-phosphocholine, is an intermediate of phosphatidylcholine (PC) metabolism, which is the main component in all eukaryotic and many prokaryotic cells. The animal and human blood plasma have an LPC content of around 20% of the total phospholipids. Lysophospholipids, LPC in particular have been shown to exhibit very good chemotactic properties to lure the immune components. LPC is classified as a second messenger that is produced by activation of cytosolic hormone-activated PLA2 (Prokazova et al., 1998). It has been shown that LPC exerts this transduction mechanism through a G2A receptor. Coupled to this receptor, LPC regulates the proliferation and differentiation of T lymphocytes and activates the release of cytokines at the site of tissue damage. The physiological role of the elevation of LPC in tissues is associated with its ability to enhance or even evoke cell proliferation, stimulate adhesion and differentiation of lymphoid cells, have mitogenic effect on macrophages activate human T-lymphocytes, initiate monocyte chemotaxis, and modulate aggregation of platelets (Lum et al., 2003).

### Materials and Methods

### Materials

Lysophosphatidylcholine in the form of Lysoforte™ booster (Kemin Industries), at 500 g/ton of feed; lutein in the form of OroGLO® brand lutein from Kemin Asia at 2 kg/ton of feed; and chromium propionate from Kemin America at 0.5 ppm. The broilers were Cobb 400, day old chicks. A commercial broiler diet, spread out into pre-starter, starter and finisher phase (Vangili Feeds). The vaccines were New Castle Disease Virus (Intermediate strain) (NDV) and Infectious Bursal Disease Virus (Lentogenic strain) (IBD). The instruments comprised V-bottom 96 well assay plate (Tarson), multichannel pipettes, agar plates, 2 ml sterile disposable syringes for blood collection, saline, sterile tubes and vials for serum separation, Phosphate buffered solution, disposable tips, dissection kit for organ extraction, and an electronic weighing balance (Sartorius).

### Methods

Experimental design: The trial was conducted on straight run commercial hybrid broiler chicks for a period of six weeks. Duration of the trial: 0-45 days Breed: Vencobb Total number of birds: 330 Treatments: 4 Control: 1 Replicates: 3 No of birds/replicate 22

Treatments Diet: Control Commercial broiler feed (PS: 15d, S: 10d, F: 17d. Treatment 1 (LPC) Control feed+Lysoforte™ booster (500 g/T) Treatment 2 (Lutein) Control feed+OroGLO® brand lutein (2000 g/T) Treatment 3 (CP) Control feed+Chromium Propionate (0.5 ppm) Treatment 4 (Combination) Control feed+LPC+Lutein+CP

### Experimental Protocol

Day old chicks were purchased from VHL. The birds were segregated randomly into 5 groups with 3 replicates for each group and each replicate containing 22 birds. The maternal antibody levels in the birds were checked on the 7th day. As the maternal antibody levels were detectable, the first vaccination of NDV was done on the 9th day. The immune response for this primary dose of NDV was tested by quantifying the titers from three birds in each replicate for every treatment.

Based on the pattern of the maternal antibody levels, the chicks were immunized with the live attenuated lentogenic strain of New Castle Disease Virus.

The birds were bled after 7 days of the primary vaccination to assess the immunoglobulin level for NDV by Haemagglutination inhibition assay. On the 16th day, the birds were vaccinated with the IBD intermediate strain and bled after 7 days to measure vaccine response to IBD by Gel precipitation technique. A booster dose for NDV was administered on 23rd day, and for IBD on 30th day and the response was assessed after 7 days respectively by using the assay methods described herewith. The immune organs, the spleen, bursa and thymus, were extracted from three birds from each replicate for every treatment on the 23rd day and 37th day. These organs were evaluated physically by relative weights. The body weight of the bird, the feed consumption and the FCR was recorded throughout the trial on a weekly basis.

TABLE 4
Experimental Protocol

| | Study schedule - Day | | | | | | | |
|---|---|---|---|---|---|---|---|---|
| Activity | 0 | 7 | 9 | 16 | 23 | 30 | 37 | 45 |
| Animal receipt and acclimatization | • | | | | | | | |
| Health check and randomization | • | | | | | | | |
| Maternal antibodies (bleeding) | | • | | | | | | |
| Control feed without AGPs - Group I | • | • | • | • | • | • | • | • |
| LPC (Feed without AGPs) (Lysoforte ™ booster 500 g/T of | • | • | • | • | • | • | • | • |
| feed) - Group II | | | | | | | | |
| Lutein 200 g/T (Feed without AGPs) - Group III | • | • | • | • | • | • | • | • |
| Chromium Propionate 0.5 ppm (Feed without AGPs) - Group | • | • | • | • | • | • | • | • |
| IV | | | | | | | | |
| LPC + Lutein + Chromium Propionate (Feed without AGPs) - | • | • | • | • | • | • | • | • |
| Group V.: (500 g/T Lysoforte booster + 200 g/T OroGLO ® | | | | | | | | |
| Brand lutein + 0.5 ppm CP) | | | | | | | | |
| NDV vaccination - primary dose | | | • | | | | | |
| Vaccination response to NDV (bleeding) | | | | • | | | | |
| IBD vaccination - primary dose | | | | • | | | | |
| Vaccination response (bleeding) for both NDV and IBD | | | | | • | | | |
| Booster dose -NDV | | | | | • | | | |
| Vaccination response (bleeding) for NDV and booster | | | | | | • | | |
| vaccination for IBD | | | | | | | | |
| Immune organs (bursa, thymus and spleen) | | | | | • | • | | |
| Vaccine response - IBD booster | | | | | | | • | |
| Mortality and clinical signs/symptoms | • | • | • | • | • | • | • | • |
| Schedule sacrifice | | | | | • | | • | |

Blood collection: Blood was collected in sterile test tubes and allowed to remain in the slanting position for serum to separate out. The separated serum was collected in vials.

Haemagglutination test for NDV: This test was performed in “V” bottom 96 well assay plate (Tarson). The virus was serially diluted (Two fold) in 50 μl of sterile saline. The agglutination was checked by adding the blood serum in the respective wells of a row. The titer was the reciprocal of the highest dilution to give complete agglutination.

Infectious Bursal Disease Virus (IBD) Antigen: The Agar Gel Precipitation Technique (AGPT) was used to confirm the virus as per the method described by Sukumar and Dorairajan (2001).

Immune organ weights: The immune organs were extracted using the dissection kit. The spleen, Bursa and thymus were extracted and the organs were weighed immediately using the digital balance. The bird weight was also noted. The relative weight of the organ was calculated thus, (Organ weight)/(Body weight of the bird in kg).

Statistical analysis: Analysis was done by one-way ANOVA using the SAS package.

### Reults and Discussion

NDV—Haemagglutination titers: The titer values obtained in the Haemagglutination assay for NDV are tabulated in Tables 5 and 6. Table 2 represents the titer values in response to the primary vaccination. The values indicate a higher response in the LPC group and in the Chromium propionate group. The titer values obtained in the combination group are lower than the response from the individual components. From the values obtained in the secondary vaccine response also it can be observed that the response is higher in the LPC group and in the chromium propionate group. In the secondary response also, the titers obtained in the combination group were lower than the other groups. From the titer values obtained it can be interpreted that the combination did not have a positive effect in the immune potentiation against ND antigen.

TABLE 5
Titer of immunoglobulin against primary ND vaccination

| | | | | | | | | | | | | | LPC + | | |
|---|---|---|---|---|---|---|---|---|---|---|---|---|---|---|---|
| | Control | | | LPC | | | Lutein | | | CP | | | Lutein + CP | | |
| R1 | 256 | 128 | 32 | 256 | 128 | 128 | 128 | 256 | 256 | 128 | 128 | 256 | 64 | 64 | 128 |
| R2 | 128 | 128 | 64 | 128 | 128 | 64 | 64 | 64 | 64 | 64 | 64 | 64 | 64 | 64 | 32 |
| R3 | 512 | 256 | 64 | 256 | 256 | | 64 | 256 | 64 | 256 | 512 | 256 | 64 | 64 | 64 |

TABLE 6
Titer of immunoglobulin against secondary ND vaccination

| | | | | | | | | | | | | | LPC + | | |
|---|---|---|---|---|---|---|---|---|---|---|---|---|---|---|---|
| | Control | | | LPC | | | Lutein | | | CP | | | Lutein + CP | | |
| R1 | 64 | 64 | 32 | 64 | 128 | 64 | 64 | 64 | 64 | 64 | 64 | 64 | 32 | 64 | 64 |
| R2 | 64 | 64 | 64 | 128 | 128 | 64 | 128 | 128 | 32 | 64 | 64 | 128 | 64 | 64 | 32 |
| R3 | 64 | 64 | 64 | 64 | 64 | 64 | 64 | 256 | 64 | 128 | 128 | 64 | 32 | 32 | 64 |

IBD—AGPT titers: The titer values obtained in the quantification of antibodies against IBD vaccine are depicted in Table 7 and Table 8. The values were not interpretive of any specific increase in the immune levels in any specific group. The titers were almost the same in all the groups.

TABLE 7
Titer values against primary IBD vaccination

| | Control | | | Treatment 1 | | | Treatment 2 | | | Treatment 3 | | | Treatment 4 | | |
|---|---|---|---|---|---|---|---|---|---|---|---|---|---|---|---|
| R1 | Log₂¹ | Log₂² | Log₂² | Log₂¹ | Log₂¹ | Log₂² | Log₂¹ | Log₂² | Log₂² | Log₂² | Log₂² | Log₂² | Log₂¹ | Log₂¹ | Log₂² |
| R2 | Log₂² | Log₂² | Log₂¹ | Log₂² | Log₂² | Log₂¹ | Log₂² | Log₂² | Log₂¹ | Log₂¹ | Log₂² | Log₂¹ | Log₂¹ | Log₂² | NA |
| R3 | Log₂² | Log₂¹ | Log₂² | Log₂² | Log₂¹ | Log₂¹ | Log₂¹ | NA | Log₂² | Log₂² | Log₂¹ | Log₂² | Log₂² | Log₂¹ | Log₂¹ |

NA: Not available

TABLE 8
Titer values against secondary IBD vaccination

| | Control | | | Treatment 1 | | | Treatment 2 | | | Treatment 3 | | | Treatment 4 | | |
|---|---|---|---|---|---|---|---|---|---|---|---|---|---|---|---|
| R1 | Log₂³ | Log₂⁴ | Log₂⁴ | Log₂² | Log₂³ | Log₂³ | Log₂³ | Log₂² | Log₂⁴ | Log₂⁴ | Log₂⁴ | Log₂² | Log₂³ | Log₂² | Log₂² |
| R2 | Log₂⁴ | Log₂³ | Log₂³ | Log₂³ | Log₂³ | Log₂⁴ | Log₂³ | NA | Log₂³ | Log₂³ | Log₂² | NA | Log₂² | Log₂² | Log₂³ |
| R3 | Log₂³ | Log₂³ | Log₂⁴ | Log₂³ | Log₂³ | Log₂² | Log₂² | Log₂³ | Log₂³ | Log₂³ | Log₂³ | Log₂³ | Log₂² | Log₂³ | Log₂³ |

NA: Not available

Overall, from the vaccine response in the levels of antibodies produced against NDV and IBD, it appears that the LPC group and the chromium propionate group have shown some positive response towards immune potentiation. Of all the groups, there has been a positive increase in these two groups.

Relative weights of immune organs: From the relative weights of the immune organs measured, it could be observed that the relative weight of the bursa was higher in the LPC treated group and the relative weight of the thymus was higher in the chromium propionate treatment group. However, the relative weight of spleen was higher in the control group in both the sampling (23rd day and 37th day). In the first sampling, the relative weight of spleen was higher in the LPC group next only to the control in comparison to all the groups.

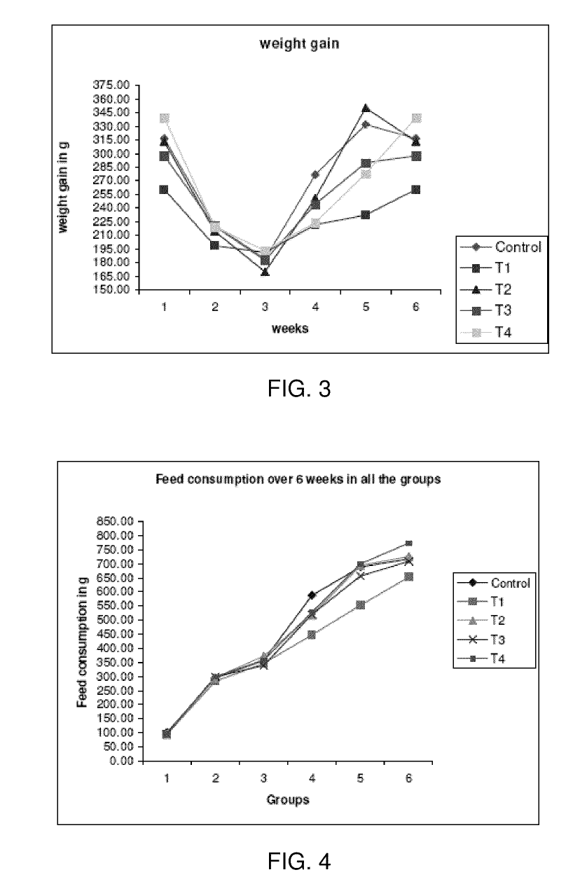

Performance parameters: The feed intake, body weight, FCR, mortality, any other signs and symptoms were monitored throughout the trial period. It was observed that the weight gain was lowest in the LPC treated group. The FCR was also found to be the highest in the LPC treated group. A similar trend was observed in the chromium propionate treatment group. The performance of the lutein group was comparable to that of the control group. FIGS. 2, 3 and 4 are representative of the performance of the birds in terms of FCR, weight gain and feed consumption respectively. FIG. 2 represents the FCR pointed on two scales, plotting the initial and the final week's FCR.

There has been an overall poor weight gain in all the birds in this trial. The decline in the weight gain was more apparent in week II and week III in all the groups. This trend could be due to the impact of heat stress during that period. Nevertheless, week II and III are considered to be the crucial stages during growth of the birds. However during the subsequent weeks, the weight gain revived in the control and the T2 groups, whereas, the trend of lowered weight gain continued to be so in the other three groups, the LPC, chromium propionate and the combination groups, throughout the trial period. Also the very high variability in the data could be due to bird sacrificing and blood sampling schedules. Pertaining to the performance parameters, the results obtained in the combination group are also quite comparable to the LPC group. It could be so because of the presence of LysoforteTm Booster in that group also. However, this similarity is not reflected in the immune response or in the immune organ weights.

The increased FCR and poor weight gain in the LPC treatment are indicative of a higher rate of metabolism in the birds. Similar results have been obtained in trials conducted so far in the application of Lysoforte™ Dry at a higher inclusion rate. In this particular trial also, the inclusion of Lysoforte™ Booster at the rate of 500 g per ton of feed is comparable to the application of Lysoforte™ Dry at the rate of 2.5 Kg/ton of feed. 62.5 g of Lysoprin+Bolec present in 100 g of Lysoforte™ booster, i.e. at the rate of 2% LPC actual in the formulation. Earlier trials conducted with Lysoforte™ at over 1 Kg/Ton application, showed a similar negative performance in terms of Feed intake, weight gain and FCR. The results obtained in this trial are also in line with this previous finding. This leads to the thought that Lysoforte™ perhaps has a bimodal effect of action in the body. If the role of Lysoforte™ was limited to that of a surfactant, then a higher level of application would have just produced a sigmoidal curve with no further enhancement in performance beyond a certain level of inclusion. But in this case, an apparent negative performance is indicative of the possibility of a bell-curve in correlation to the inclusion level and performance. This has to be further validated by conducting dose response studies with the inclusion of LPC (Lysoforte™) at different levels, and the ideal inclusion rate for the maximum performance and enhanced immune potentiation will have to be determined. Overall from the trials as it could be observed that the HI titers for NDV and the immune response for IBD, measured during the trial period have shown an increased response in the T1, T3 and control groups and from the relative weights of the immune organs, also measured during that period, it follows that the spleen and bursal weights were higher for the T1 group and control, whereas, the weight of thymus was higher in the T3 group. The effect of LPC and chromium propionate has thus shown a tendency towards potentiating immunity in birds. Prior studies have recorded the chemotactic properties of LPC. However the inclusion level being so low in the current treatment, it appears to be that the functional attribute is expressed at a therapeutic level where it acts through the binding with G2A receptor. The G2A mediated activities of LPC are being explored through many in vitro experiments elsewhere. It could also be possible that LPC acts through the G2A receptor in mediating the lipid metabolism in vivo. It has also been described that LPC is found attached to the oxidized LDL. This is suggestive of the possible mechanism of action of LPC, mediated through its binding to the G2A receptor, to bring about lipid catabolism at specific levels of inclusion. This could be possible through its action on the signal transduction; by its G-a subunit binding to the s or the q subunit it could specifically mediate the increase in the calcium levels or the camp levels respectively. G-protein-coupled receptors (GPCRs) mediate various cellular functions including proliferation, differentiation, adhesion, and migration and play pivotal roles in development, homeostasis, inflammation, immunity, oncogenesis, and cancer metastasis (1-5) G2A is expressed in pro-B and T cells, and the expression of G2A is inducible upon activation and stress stimulation in the cells (6,7). G2A is an immunoregulatory G protein-coupled receptor predominantly expressed in lymphocytes and macrophages. Ectopic over expression studies have implicated G2A as a receptor for the bioactive lysophospholipid, lysophosphatidylcholine (LPC). However, the functional consequences of LPC-G2A interaction at physiological levels of receptor expression, and in a cellular context relevant to its immunological role, remain largely unknown. (8).

### CONCLUSION

From the results of this screening experiment to determine the effect of LPC, lutein and chromium propionate, individually and in a combination to bring about Immune potentiation in birds, it has been inferred that the LPC treated group has shown a tendency towards increased immune response. From the results it was also inferred that the combination did not enhance the immunity.

### REFERENCES

1. Fromm, C., Coso, O. A., Montaner, S., Xu, N., and Gutkind, J. S. (1997). The small GTP-binding protein Rho links G protein-coupled receptors and Ga 12 to the serum response element and to cellular transformation. Proc. Natl. Acad. Sci. U.S.A. 94, 10098-10103.
2. Luther, S. A., and Cyster, J. G. (2001). Chemokines as regulators of T cell differentiation. Nat. Immunol. 2, 102-107.
3. Chemin, J., Patel, A., Duprat, F., Zanzouri, M., Lazdunski, M. and Honoré, E. (2005). Lysophosphatidic Acid-operated K+ Channels. J. Biol. Chem., 280: Issue 6, 4415-4421.
4. Ishii, I. Fukushima, N., Ye, X. and Chun, J. (2002), Lysophospholipid Receptors: Signaling and Biology. Annual Review of Biochemistry, 73: 321-354.
5. Anliker B. and Chun, J. (2004). Lysophospholipid G Protein-coupled Receptors. J. Biol. Chem., 279: 20555-20558.
6. Weng, Z., Fluckiger, A.-C. Nisitani, S. Wahl, M. I. Le, L. Q. Hunter C. A., Fernal, A. A., Le Beau, M. M. and Witte, O. N. (1998). A DNA damage and stress inducible G protein-coupled receptor blocks cells in G2/M.; Proc. Natl. Acad. Sci. Cell Biology, 95(21): 12334-12339.
7. Caius, G. Li, Radu, Yang, V., Riedinger, M., Au, M. and Witte, O. N., (2003) T cell chemotaxis to lysophosphatidylcholine through the G2A receptor. PNAS, Proc. Natl. Acad. Sci., Jan. 6, 2004, vol. 101, no. 1, 245-250
8. Soon, D. (2002). Orphan G Protein-Coupled Receptors and Beyond. J. Pharmacol. 90: 101-106
9. Chew, B. P. Park, J. S., Wong, T. S., Kim, H. W., Brian, B. C. Weng, Byrne, K. M., Hayek, M. G. and Reinhart, G. A. (2000). Dietary B-Carotene Stimulates Cell-Mediated and Humoral Immune Response in Dogs. Journal of Nutrition. 130: 1910-1913.
10. Luml, H., Qiao, J., Walter, R. J., Huang, F., Subbaiah, P. V., Kim, K. S., and Holian, O. (2003). Inflammatory Stress Increases Receptor for Lysophosphatidylcholine in Human Microvascular Endothelial Cells. Am J Physiol Heart Circ Physiol 285: HI756-HI789,June 2003.
11. Prokazova, N. V., Zvezdina, N. D., Korotaeva, A. A., (1998). Effect of Lysophosphatisylcholine on transmembrane signal transduction. Biochemistry (Mosc). January: 63(1): 31-37.
12. Sukumar K and Dorairajan N. Hemagglutination and Hemagglutination Inhibition tests for assessment of antibody levels against New Castle Disease. Compendium. Winter school conducted by Veterinary College and Research Institute (VC&RI), Namakkal, India. Molecular Diagnosis and Modern trends in the control of poultry, diseases. 2001 December 162-164.

### EXAMPLE 3

### Effect of Lysophospholipids, More Particular Lysophosphatidylcholine (LPC) on Inflammatory Bowel Disease

The prokaryotic and eukaryotic cells of the colon exist in a highly complex, but harmonious relationship. Disturbances in this remarkable symbiosis result in the development of inflammatory bowel diseases (IBD).Although the etiology of IBD is not entirely understood, it is known that the chronic inflammation of Crohn's disease and ulcerative colitis are a result of overly aggressive immune response to the commensal intestinal flora in genetically susceptible hosts. Despite having a common basis in over responsiveness to mucosal antigens, the two diseases have considerably different pathophysiologies. Crohn's disease is associated with a Th1 T cell-mediated response induced by IL-12 and possibly IL-23, whereas ulcerative colitis is associated with an atypical Th2-mediated response characterized by NKT cell secretion of IL-13 in humans. However, animal models of colitis have yielded evidence of both Th1- and Th2-mediated conditions.

TNBS induced rat model is widely accepted for the study of colon inflammation and its treatment. The major role proinflammatory cytokines such as IL-1 β, IL-6, TNF-α and IFN-γ play in the pathogenesis of IBD is well established. A pro-inflammatory cytokine mediated inflammatory response is a cascade of gene products generally not evident in healthy individuals. Pro-inflammatory genes, including phospholipase A2 (PLA2), cyclooxygenase (COX)-2, and inducible NO synthase code for enzymes that increase the production of platelet-activating factor and prostanoids, leukotreines, and NO. These small mediator molecules are up-regulated during inflammation. IL-1β and TNF act synergistically in stimulating the expression of these genes. Anti-inflammatory cytokines can mediate effects that down-regulate inflammatory responses thereby suppressing the intensity of the cascade. Cytokines such as IL-10 and transforming growth factor (TGF)-b restrains the production of IL-1β and TNF-α. Several studies have shown that elevated levels of TNF-α in particular, feature strongly in IBD Therapeutic agents are being developed to limit or neutralize the action of agents involved in the inflammatory process.

### Experiment 1: Using Lysolecithin as a Natural Source of LPC

Experiments were conducted using TNBS induced colitis model in rats. The experiments were conducted in 10-12 weeks old male Wistar rats for a period of 10 days. Colitis was induced by intracolonic instillation of TNBS at a dose of 100 mg/kg body weight. Forty two animals were divided into seven groups with 6 animals in each group: 1) Positive Control sacrificed on day 4; 2) Positive control sacrificed on day 11; 3) Negative control (saline instillation); 4) Positive control treated with PB6 at 1.5 10⁸ CFU/Kg body weight (thrice daily from day 4-10) 5) Positive control treated with Lysophospholipids at 21.5 mg/kg body weight (twice daily from day 4-10) 6) Positive control treated with a combination of Lysophospholipids, Omega-3 fatty acid and Carotenoids at 21.5 mg /kg body weight (twice daily from day 4-10); 6) Positive control treated with Mesacol® (5-aminosalicylic acid) at 250 mg/kg body weight (once daily from day 4-10).

Body weights were recorded on day 1, 4, 7 and 10. Blood sampling was done on day 4 (groupl and 3) and on day 7 and 11(all other groups) for determining the plasma levels of IL1β, IL6, and IL10, TNFα, IFNγ and TGFβ. The colon were collected on day 4 (group1) and on day 11 (all other groups) for scoring of gross morphology and colon weight.

### Materials and Methods

### Materials

The source of mesalazine was Mesacol® 5-amino salicylic acid tablets obtained from Sun Pharmaceutical Industries. The source of 2,4,6 -trinitrobenzene sulfonic acid was Sigma-Aldritch as picrylsulfonic acid solution, 5% (w/v). The source of lysophospholipids was Lysoprin™ from Kemin Industries, Inc. The source of omega-3 fatty acids was C38 Powder from Loder's Croklaan. The source of lutein was FloraGLO® brand lutein from Kemin Industries, Inc. The source of Bacilis subtilis PB6 was PB6 dry concentrate FG from Kemin Health, L.C.

### Preparation and Administration

Mesalazine (5-aminosalicyclic acid), the active component in Mesacol®, is widely used for the treatment of IBD (Small et al., 1994) and therefore acted as a positive comparative treatment. Tablets were powdered and suspended in an appropriate volume of distilled water to attain the concentrations of 25 mg/ml. A dose of 250 mg/kg/day was administered orally using an 18G feeding tube attached to appropriately graduated syringe, starting on day four until day ten. A dose volume of 10 ml/kg was employed. The dose of the drug was selected based on its ability to exert significant anti-inflammatory effects and patently decrease proinflammatory cytokines IL-1, IL-8 and TNF-α (Mao-Mao et al., 2005).

2,4,6 -Trinitrobenzene sulfonic acid was diluted with 50% ethanol (v/v) to attain the dilution to 25 mg/ml TNBS. TNBS was administered on day 1 by intrarectal route using a 2 ml syringe attached to a 10 cm polyethylene catheter at a dose of 100 mg/kg. A dose volume of 4 ml/kg bodyweight was employed.

The combination treatment consisted of Lysoprin as a source of lysophospholipids, omega-3 fatty acids powder as a source of omega-3 fatty acid and FloraGLO® brand lutein as a source of carotenoids. Due to the identified dose-response relationship of this combination treatment in the trials with ulcerative colitis rats, the same combinations were used in this trial. The dose consisted of 10.3 mg/kg lysophospholipids, 10.3 mg/kg omega-3 fatty acids, and 5.1 mg/kg of carotenoids. The dose was administered thrice daily beginning on day four, after the evidential effects of TNBS-induction, and continued up to and including day ten. The components were blended well to accomplish the concentration of 2.15 mg/ml. The combination of these compounds was water insoluble, thus to maximize the drug potential, distilled water was used as a diluents to emulsify the lipids. The combinations were stirred with 20 ml of distilled water as an emulsion for 10 ml/kg body weight, and vigorously shaken. Doses were prepared daily twice and used immediately. An 18G feeding tube affixed to a suitably adjusted syringe was utilized to employ 10 ml/kg of the drug. The dose of administration was arrived at based on the effective employment of the test item in the previous trials with Wistar rats in the treatment of inflammation associated with ulcerative colitis. (Selvam et al., unpublished).

Lysoprin as a source of lysophospholipids was administered orally by using 18G feeding tube attached to appropriately graduated syringe at a dose of 10.3 mg/Kg/day starting on day 4 and up to and including day 10. Dose volume employed was 10 ml/kg body weight.

The required quantity of PB6 for the test item were weighed and an appropriate volume of distilled water was added and mixed well to attain concentrations of 1.5 mg/ml. Doses were prepared daily twice and dosed starting on day 4 and up to and including day 10.

### Study Design

The study was conducted with all relevant federal guidelines and institutional policies. Thirty six Male Wister rats, aged 10-12 weeks and weighing 180-200 g were obtained from the National Centre for Laboratory Animal Sciences, Hyderabad, India. Animals were housed for one week before the onset of the experiment and acclimatized under standard laboratory conditions at a constant temperature of 19-25° C., with a relative humidity of 30-70%, and a 12-h light/dark cycle (Johnson & Besselsen, 2002). Each cage contained three animals (Donnelly, 1995), with readily available rat pellet food and filtered water ad libitum throughout the study, with the exception of the twelve hours before the initiation of the trial. Veterinary health checks were carried out during acclimatization; the animals were randomly categorized into seven groups (1 to 7).

Group 1 (n=6) were induced with TNBS colitis and was sacrificed three days following instillation to determine the resulting degree of colonic damage. The administration of TNBS in untreated rats in group 2 (n=6) provided a drug-free TNBS-positive control group. Group 3 (n=6) received saline and served as vehicle controls to determine baseline levels of different parameters and for comparison with TNBS Group 4 (n=6) were induced with TNBS-colitis and received PB6. Group 5 (n=6) were induced with TNBS-colitis and received lysophospholipids. Group 6 (n=6) were induced with TNBS-colitis and received a combination of lysophospholipids, omega-3 fatty acid and carotenoids and Group 7 (n=6) was induced with TNBS and received a standard treatment drug Mesacol®. The mean weight variation between the groups was minimal and did not exceed ±20%. The use of identification numbers, cage cards, and animal marking allowed the recognition of animal groups. All experimental protocols described in this report were approved by the Institutional Animal Ethics Committee in accordance with the procedures specified by the Committee for the Purpose of Control and Supervision of Experiments on Animals, regarding animal care and handling. All modus operandi were strictly followed the standard operating procedures of the laboratory and good laboratory practice was maintained at all times. The details of treatment groups, dosage and concentration given are detailed in Table 9.

TABLE 9
Details of treatment groups, dosage and concentration

| Dose Group | Dose | | Concentration | |
|---|---|---|---|---|
| TNBS Baseline Group. | 4 | ml/kg | 25 | mg/ml |
| TNBS induced. No treatment. | | | | |
| TNBS Positive-Control Group. | 10 | ml/kg | — | |
| TNBS induced. Distilled water given orally as a | | | | |
| control vehicle thrice/day from day 4-10. | | | | |
| TNBS Negative Group (Saline). | 10 | ml/kg | — | |
| Saline induced. Distilled water given orally as a | | | | |
| control vehicle thrice/day from day 4-10. | | | | |
| PB6 Treatment Group | 4 | ml/kg | 25 | mg/ml |
| TNBS induced. Pb6 given orally thrice/day from day 4-10. | 1.5 10⁸ | CFU/Kg | 1.5 10⁷ | CFU/ml |
| | (10 | ml/kg) | | |
| Lysophospholipids treatment Group | 4 | ml/kg | 25 | mg/ml |
| TNBS induced. Lysophospholipids given orally twice/ | 10.3 | mg/Kg | 1.03 | mg/ml |
| day from day 4-10. | (10 | ml/kg) | | |
| Combination of Lysophospholipids, Omega-3 Fatty | 4 | ml/kg | 25 | mg/ml |
| Acids, Carotenoids Group | 22 | mg/Kg | 2.2 | mg/ml |
| TNBS induced. Combination of Lysophospholipids, | (10 | ml/kg) | | |
| Omega-3 Fatty Acids, Carotenoids orally twice/day | | | | |
| from day 4-10. | | | | |
| Standard Treatment Group (Mesacol ®) | 4 | ml/kg | 25 | mg/ml |
| TNBS induced. Mesacol ® given orally once/day from | 250 | mg/kg | 25 | mg/ml |
| day 4-10. | (10 | ml/kg) | | |

The animals were weighed after randomization, day 1, day 4, day 7 and day 11. The weight gains were calculated with this data on day 4, day 7 and day 11.

### Blood Collection

Blood samples were taken through the retro-orbital plexus to quantify the cytokine response to treatment. Samples were collected from treated and control rats on days 4, 7 and 11 after colitis was induced. Following general anesthesia, 5 -8 ml of blood was collected through retro-orbital plexus puncturing in heparinised 15 ml centrifuge tube and centrifuged at 2000 rpm for 10 minutes for separation of plasma. Sequential to the separation of the plasma, a micropipette was used to collect the plasma in vials and was stored at −80° C. until analysis for cytokines.

### Determination of Colonic Inflammation

To determine the early effects of TNBS, group1 rats were sacrificed on day 4 after the stimulation of inflammation. The remaining animal groups were sacrificed on day eleven to assess macroscopic colonic changes. A 5 cm segment of distal colon was removed from each animal, opened longitudinally, and fecal material was cleaned using ice-cold saline. It was then blotted against a blotting paper and examined by one independent observer blinded to the treatment. The colon samples were weighed, and the wet weight of the segment was determined as a reflection of colonic edema. Following scoring, the colons were blotted dry and again weighed. The colon weight:body weight ratio was quantified, as an indicator of colonic inflammation (Rolandelli et al., 1988).

The time points chosen to sacrifice the animals were considered illustrative of acute and chronic inflammation that can be induced in the TNBS model. Previous studies have indicated that a time frame of three days is needed to determine colonic inflammation (Whittle et al., 2006). The levels of neutrophil infiltration and acute tissue inflammation, following TNBS caused damage, plateau at this point (Boughton-Smith et al., 1988a,b). A monocytic/lymphocytic infiltration is evident after eleven days combined with substantial neutrophils and eosinophils near ulcers (Morris et al., 1989). Furthermore, many studies investigating the effects of experimental drugs in the TNBS model have used the 11-day time point (Wallace and Keenan, 1990; Woodruff et al., 2005).

### Macroscopic Examination of Intestinal Damage

The extent of mucosal damage was assessed macroscopically according to the scale described by Wallace and Keenan, 1990, which included the area of inflammation and the presence or absence of ulcers. Each colon was assigned a score on this scale ranging from 0 to 5 (Table 10) indicative of areas of mucosal discoloration, erosion, exudation, ulceration, and bowel wall thickening. The degree of intestinal damage was assessed by a naked-eye examination immediately after the scarification of the animals. The inflammatory response was measured as both area of involvement of the colonic segment and its severity as a macroscopic score.

TABLE 10
TNBS Colitis Macroscopic Grading Standards.

| Grade | Finding |
|---|---|
| 0 | No damage |
| 1 | No ulceration, localized hyperaemia |
| 2 | Ulceration with no significant inflammation |
| 3 | Ulceration with inflammation at one site |
| 4 | 2 or more sites of inflammation and/or ulceration |
| 5 | 2 or more major sites of inflammation and/or |
| | ulceration or 1 major site of inflammation and |
| | ulceration extending 2 cm along the colon. |

### Measurement of Cytokine Concentration in the Blood

The effects of the test items on plasma cytokine levels as inflammatory biomarkers were evaluated using ELISA development kits and levels were expressed as pico-grams per milliliter. The cytokines analyzed were IL1-β, IL6, IL10, TNF-α, IFN-γ and TGF-beta. The cytokines measured were approved based on their substantial association with the inflamed mucosa of patients with IBD (Martinez-Borra, 2002; Maerten et al., 2004) and TNBS-induced colitis in the rat (Villegas et al., 2003).

### ELISA Procedure

The DuoSet ELISA development kits were utilized to quantify cytokine concentrations in various groups. The ELISA (Enzyme Linked Immunosorbent Assay) technique was based on the antibody sandwich principle, a sensitive enzyme immunoassay that can specifically identify and enumerate the concentration of soluble cytokine proteins. Assays were performed according to the instructions provided with each kit. In brief, a capture antibody specific to the analyte of interest was bound to a plastic microwell plate overnight to create the solid phase. After binding of the antibody to the well, the plate was washed and coated with a non-reactive material to reduce background interference. The plate was washed again to remove unbound material and the immobilizing surface was contacted with the samples, standards and controls to be tested under conditions effective to allow immune complex (antigen/antibody) formation. This incorporated the incubation of the samples, standards and control with the solid phase antibody, which captured the analyte. Unbound analyte was washed away and a biotin-conjugated antibody was added to detect the captured cytokine proteins. A detection reagent (stretavidin-HRP) was also added, following a wash to remove unbound detection antibody. The plate was washed for the final time, a substrate solution was added and color developed in proportion to the amount of bound analyte. Color development was stopped and the optical densities (OD) were measured using SPECTRAmax 190 microplate spectrophotometer at a wavelength of 450 nm. Data were analyzed against the linear portion of the generated standard curve of the corresponding recombinant rat cytokine.

The ELISA development kits had been standardized and validated prior to the quantification of the cytokines. Serial dilutions of the standard cytokine protein solutions were prepared at known concentrations in order to obtain a standard curve. This provided assurance that the OD fell within the linear portion of the standard curve. Different levels of dilution were tested to provide flexibility to the assay samples with different levels of analyte. The employment of the spike-and-recovery experiment verified whether analyte detection was influenced by a difference between the diluent used to prepare the standard curve and the biological sample matrix. The lower limit of detection of ELISA was 31.25 and 16.125 (pg/ml) for TNFα and IL-10 respectively. All interassay and intraassay coefficients of variation were less than 10%. Ambiguous results were repeated.

### Statistical Analysis

The data were subjected to one-way analysis of variance (ANOVA) and Duncan's multiple range test using Statgraphics plus 5® (StatPoint, Inc.; 2000) and all statements of significance are based on the 0.05 and 0.01 level of probability.

### Results and Discussion

A study was conducted investigating the effect of a combination of lysophospholipids, more particular lysophosphatidylcholine (LPC), omega-3 fatty acids and carotenoids, lysophospholipids and PB6 on the TNBS model of colitis. The TNBS rat model is widely accepted for the study of colon inflammation and its treatment and is therefore described as the model of choice in this study. In the present study the effect of combination of lysophospholipids, omega-3 fatty acids and carotenoids, lysophospholipids and PB6 was investigated in the control of inflammation in TNBS induced colitis rats. The test items have previously been found to provide beneficial effects in the treatment of inflammation in Wistar rats in TNBS induced colitis model.

The results obtained showed that TNBS induced colitis animals (positive control) displayed a severe diarrhea, weight loss and a severe colonic inflammation characterized by mucosal edema, ulceration, erosion and adhesion. Prior to treatment, all animals administered with TNBS-colitis experienced significant weight loss compared with saline-control rats (p<0.05) during day 1 to 4. Among treatment groups, all animals showed weight loss including the saline treated groups during day 4 to 10 except PB6 treated group which showed weight gain but of insignificance. Although treatment with Mesacol® reduced the severity of inflammation to some degree, it was not as effective as the PB6 treated group.

The saline group demonstrated significantly (p<0.01) lower colon weights in comparison to the TNBS positive control groups. All the other treatment groups also demonstrated significantly (p<0.01) lower colon weights in comparison to the TNBS positive control group. Even though there was no significant difference between the treatment groups, the PB6 treated group showed improvement on the wet colon weight, followed by combination of lysophospholipids, omega-3 fatty acid & carotenoids, Mesacol® and lysophospholipids alone. The colons of saline control vehicles showed no macroscopic lesions at all. Among the treatment groups, PB6, combination of lysophospholipids, carotenoids and omega-3 fatty acid, lysophospholipids and Mesacol® resulted in a significant improvement in macroscopic scores compared with drug-free colitis control rats. However, the PB6 treated group showed the least colon score, followed by the combination of lysophospholipids, omega-3 fatty acid and carotenoids and Mesacol® which showed same colon scores and finally lysophospholipids alone. Among the treatment groups, there was significant difference between lysophospholipids treated and the other treatment groups.

Macroscopic examination of inflamed lesion specimens of the two TNBS colitis control groups demonstrated severe damage, with obvious mucosal injury and inflammation, including hyperemia and swollen tissue. The saline control groups showed no macroscopic lesions in the colon. The PB6 at 1.5 10⁸ CFU/kg treated groups showed no visible inflammation or injury in the colonic tissue. The lysophospholipids given at 10.3 mg/kg showed visible inflammation and injury in few colonic tissues but of insignificance compared to TNBS control. The combination of lysophospholipids, carotenoids & omega-3 fatty acid at 21.63 mg/kg showed no visible inflammation or injury in the colonic tissue The Mesacol® group showed no lesions with any visible inflammation or injury in the colonic tissue.

The plasma levels of TNF-α were high in TNBS induced control vehicle than the saline instilled group on day 7 and significantly high (p<0.05) on day 11. Among the treatment groups, only Mesacol® showed significant reduction (p<0.05) in TNF-α. In day 11 only PB6 showed significant reduction (p<0.05) in the plasma TNF-α.

The plasma levels of IL1-β were high in TNBS induced control vehicle than the saline instilled group in day 7 and significantly high (p<0.05) in day 11. Among the treatment groups, PB6 showed significant reduction in day 7 and 11. Lysophospholipids and combination of lysophospholipids, omega-3 fatty acids and carotenoids showed reduction in day 7 and significant reduction (p<0.05) in day 11. Mesacol® showed no improvement in the plasma IL1-β levels.

There was no change in the plasma levels of IL 6 among TNBS induced control vehicle and the saline instilled group and other treatment groups on day 7. However on day 11, the combination of lysophospholipids, omega-3 fatty acids and carotenoids and the Mesacol® treated groups showed significantly decreased levels (p<0.05) of IL 6. This showed that the drug treatment required a minimum of 7 days for its effectiveness in improving the cytokine levels and elicit the inflammatory mechanism.

The plasma levels of IFN-γ were significantly high (p<0.05) in TNBS induced control vehicle than the saline instilled group in day 4, 7 and 11. Among the treatments, all the groups showed significant reduction in day 7. On day 11 only PB6 showed reduction in plasma IFN-γ and Mesacol® showed a higher level than TNBS control.

The plasma level of IL 10 was higher in saline instilled rats than TNBS induced control vehicle but of insignificance in day 7 and 11. The levels of plasma IL 10 was significantly higher (p<0.05) in PB6 treated group in both day 7 and 11 than TNBS induced control vehicle. However in day 11, the standard drug Mesacol® treated group showed significantly highest (p<0.05) value of IL10 than TNBS induced control and saline instilled group.

Clinical and Physiological Changes of Rats Induced with TNBS-Colitis

The administration of TNBS in 50% ethanol resulted in the development of mild diarrhea, rectal bleeding and increased mucus within hours of instillation. Anorexia, weight loss and reduced activity ensued, and were most progressive during the first three days. The hunchback appearance of the majority of animals for 48 hours post TNBS-induction, which is an indication of severe abdominal pain, continued for the TNBS positive control group throughout the study. The severe weight loss witnessed in animals induced with TNBS-colitis mirrors human development of IBD (Podolsky et al., 2002), with prominent features including increased resting energy expenditure, body weight loss and likelihood of anorexia, especially during acute stages (Sawczenko and Sandhu, 2003). Morris and colleagues (1989) demonstrated significant correlations between weight loss and anorexia with increases in the severity of colitis. The occurrence of diarrhea associated with all TNBS groups also corresponded with the literature on IBD (Bell and Kamm, 2000). Zareie and colleagues (2001) provided evidence that physiological epithelial ion transport and transepithelial electrical resistance were altered through the activation of macrophages by bacteria and their products. A combination of lysophospholipids, omega-3 fatty acids and carotenoids was markedly effective in reducing the degree of diarrhea among the rats with TNBS-induced colitis. The saline controls demonstrated normal bowel function, consistent weight and activity throughout the trial.

Animal Performance with Respect to Body Weight and Changes

The % body weight gain/loss is given in Table 11. Prior to treatment, all animals administered with TNBS-colitis experienced significant weight loss compared with saline-control rats (p<0.05) during day 1 to 4. The animals fed with PB6 at 1.5×108 CFU/Kg orally thrice daily from day 4 up to and including day 10 showed weight gain that was significant (p<0.05). Although treatment with Mesacol® reduced the severity of inflammation to some degree, it was not as effective as the PB6 treated group.

TABLE 11
% Body weight gain/loss (Day 1-4 & day 7-10)

| | % Body weight | |
|---|---|---|
| | gain/loss | |
| Treatment Details | Day 1-4 | Day 4-10 |
| TNBS intrarectally day 1 (Sacrificed on day 4) | −6.5 | |
| TNBS intrarectally day 1 + Control Vehicle | −6.9 | −3.37 |
| (Distilled water) orally/thrice daily from day 4-10 | | |
| Saline intrarectally day 1 + Control Vehicle | 4.1 | 0.04 |
| (Distilled water) orally/thrice daily from day 4-10 | | |
| TNBS intrarectally day 1 + PB6 1.5 10⁸ CFU/Kg | −6.2 | 2.88 |
| orally/thrice daily from day 4-10 | | |
| TNBS intrarectally day 1 + Lysophospholipids | −6.1 | −0.65 |
| 10.3 mg/Kg orally 2 times daily from day 4-10 | | |
| TNBS intrarectally day 1 + Lysophospholipids + | −7.5 | 0.20 |
| Omega-3 fatty acids + Carotenoids combination | | |
| 25.75 mg/ml orally 2 times daily from day 4-10 | | |
| TNBS intrarectally day 1 Mesacol 250 mg/Kg orally | −6.6 | −1.87 |
| once daily from day 4-10 | | |

Morphological and Macroscopic Changes in Colon of Rats Induced with TNBS Colitis

The degree of oedema determined by the colon weight and the degree of macroscopic lesions in the colon as colon score is given in Table 12. The saline group demonstrated significantly (p<0.01) lower colon weights in comparison to the TNBS positive control group. All the other treatment groups also demonstrated significantly (p<0.01) lower colon weights in comparison to the TNBS positive control group. Even though there was no significant difference between the treatment groups, the PB6 treated group showed improvement on the wet colon weight, followed by lysophospholipids, omega-3 fatty acid and carotenoids combination, Mesacol® and lysophospholipids. The colons of saline control vehicles showed no macroscopic lesions at all. Among the treatment groups, PB6, combination treatment of lysophospholipids, carotenoids and omega-3 fatty acid, lysophospholipids alone and Mesacol® resulted in a significant improvement in macroscopic scores compared with drug-free colitis control rats. However, the PB6 treated group showed the least colon score, followed by lysophospholipids, omega-3 fatty acids, carotenoids combination and Mesacol® which showed same colon scores and finally lysophospholipids treated groups. Among the treatment groups, there was significant difference between lysophospholipids treated and the rest treatment groups.

TABLE 12
Colon weight and colon score

| | Colon wet | Colon |
|---|---|---|
| Dose group | weight (g) | Score |
| TNBS intrarectally day 1 (Sacrificed on day 4) | 1.207^{b} | 4.167^{c} |
| TNBS intrarectally day 1 + Control Vehicle | 1.287^{b} | 3.667^{c} |
| (Distilled water) orally/thrice daily from day 4-10 | | |
| Saline intrarectally day 1 + Control Vehicle | 0.452^{a} | 0.000^{a} |
| (Distilled water) orally/thrice daily from day 4-10 | | |
| TNBS intrarectally day 1 + PB6 1.5 108 CFU/Kg | 0.570^{a} | 0.667^{a} |
| orally/ thrice daily from day 4-10 | | |
| TNBS intrarectally day 1 + Lysophospholipids | 0.690^{a} | 2.000^{b} |
| 10.3 mg/Kg orally 2 times daily from day 4-10 | | |
| TNBS intrarectally day 1 + Lysophospholipid + | 0.663^{a} | 0.833^{a} |
| Omega-3 fatty acids + Carotenoids combination | | |
| 25.75 mg/ml orally 2 times daily from day 4-10 | | |
| TNBS intrarectally day 1 Mesacol 250 mg/Kg orally | 0.669^{a} | 0.833^{a} |
| once daily from day 4-10 | | |

The intracolonic administration of TNBS consistently produced severe colonic damage at three days post induction that continued until day 11. Macroscopic examination of inflamed lesion specimens of the two TNBS-colitis control groups demonstrated widespread damage, with obvious mucosal injury and inflammation, including hyperemia and swollen tissue. A large number of distal colon segments appeared thickened, exposing a deep reddish purple color with many hemorrhagic erosions and linear ulceration in the entire colonic wall in some cases. Damage scores were high in both groups with a mean score of 4.2 and 3.7 for TNBS baseline and TNBS positive control groups respectively (Table 12). The inflammatory response is a collective intricate series of events induced by tissue damage. The multifaceted sequences of interactions that initiate these episodes comprise of various chemical mediators, albeit with inadequately understood relations. During the early phase of inflammation, an excessive quantity of interleukins and lipid-mediators act significantly in the pathogenesis of intestinal dysfunction. The membrane phospholipids release arachidonic acid (AA), resulting in the metabolism of pro-inflammatory prostaglandins (PGs) and leukotrienes. The bioactive mediators produced in response to inflammatory stimulus, such as prostanoids, cytokines and chemokines exhibit complex, pleitropic effects and interact with many cell types to magnify the inflammatory response. The deregulation of these processes may determine acute and chronic inflammatory disease.

The saline control groups showed no macroscopic lesions in the distal colon.

The PB6 treated groups (Group 4) showed no visible inflammation or injury in the colonic tissue. The Lysophospholipid given at 10.3 mg/kg (Group 5) showed visible inflammation and injury in the colonic tissue. The combination of lysophospholipids, omega-3 fatty acids and carotenoids at 21.63 mg/kg (Group 6) showed no visible inflammation or injury in the colonic tissue. The Mesacol® group (Group 7) showed no lesions with any visible inflammation or injury in the colonic tissue.

Changes in the Cytokine Levels in Plasma of Rats Induced with TNBS Colitis

The changes in the pro inflammatory cytokines such as TNF-α and IL1-β are given in Table 13. The plasma levels of TNF-α were high in TNBS induced control vehicle than the saline instilled group in day 7 and significantly high (p<0.05) on day 11. Among the treatment groups, only Mesacol® showed significant reduction (p<0.05) in TNF-α. In day 11 only PB6 showed significant reduction (p<0.05) in the plasma TNF-α.

The plasma levels of IL1-β were high in TNBS induced control vehicle than the saline instilled group in day 7 and significantly high (p<0.05) in day 11. Among the treatment groups, PB6 showed significant reduction in day 7 and 11. Lysophospholipids and combination of lysophospholipids, omega-3 fatty acids and carotenoids showed reduction in day 7 and significant reduction (p<0.05) in day 11. Mesacol® showed no improvement in the plasma IL1-β levels.

TABLE 13
Pro inflammatory cytokines (TNF-α and IL1-β) in Plasma

| | Cytokine levels in Plasma (picogram/ml) | | | | | |
|---|---|---|---|---|---|---|
| | TNF-α | | | IL1-β | | |
| Treatment details | Day 4 | Day 7 | Day 11 | Day 4 | Day 7 | Day 11 |
| TNBS intrarectally day 1 | 35.83^{a} | | | 942.5 | | |
| (Sacrificed on day 4) | | | | | | |
| TNBS intrarectally day 1 + | | 36.61^{b} | 34.38^{b} | | 934.58^{b} | 966.80^{c} |
| Control Vehicle (Distilled | | | | | | |
| water) orally/thrice daily from | | | | | | |
| day 4-10 | | | | | | |
| Saline intrarectally day 1 + | 30.05^{a} | 30.22^{ab} | 21.05^{a} | 530.7 | 649.44^{ab} | 566.94^{a} |
| Control Vehicle (Distilled | | | | | | |
| water) orally/thrice daily from | | | | | | |
| day 4-10 | | | | | | |
| TNBS intrarectally day 1 + PB6 | | 31.66^{b} | 21.22^{a} | | 561.66^{a} | 604.44^{ab} |
| 1.5 108 CFU/Kg orally/thrice | | | | | | |
| daily from day 4-10 | | | | | | |
| TNBS intrarectally day 1 + | | 34.88^{b} | 30.83^{b} | | 737.91^{ab} | 695.83^{ab} |
| Lysophospholipid 10.3 mg/Kg | | | | | | |
| orally 2 times daily from day 4-10 | | | | | | |
| TNBS intrarectally day | | 33.27^{b} | 26.16^{ab} | | 740.13^{ab} | 631.66^{ab} |
| 1 + Lysophospholipid + Omega-3 | | | | | | |
| fatty acids + Carotenoids | | | | | | |
| combination 25.75 mg/ml orally | | | | | | |
| 2 times daily from day 4-10 | | | | | | |
| TNBS intrarectally day | | 22.77^{a} | 30.27^{b} | | 881.52^{b} | 848.33^{bc} |
| 1 Mesacol 250 mg/Kg orally | | | | | | |
| Once daily from day 4-10 | | | | | | |

Values within the column are statistically significant (p < 0.05)

The changes in the pro inflammatory cytokines such as IFN-γ and IL-6 are given in Table 14.

There was no change in the plasma levels of IL 6 among TNBS induced control vehicle and the saline instilled group and other treatment groups in day 7. However in day 11, the lower dose combination and the Mesacol® treated groups showed significantly decreased levels of IL 6. This showed that the drug treatment required a minimum of 7 days for its effectiveness in improving the cytokine levels and elicit the inflammatory mechanism.

The plasma levels of IFN-γ were significantly high (p<0.05) in TNBS induced control vehicle than the saline instilled group in day 4, 7 and 11. Among the treatments, all the groups showed significant reduction in day 7 and in day 11 only PB6 showed reduction in plasma IFN-γ and Mesacol® showed a higher level than TNBS control.

TABLE 14
Pro inflammatory cytokines (IL6 and IFN-γ) in Plasma

| | Cytokine levels in Plasma (picogram/ml) | | | | | |
|---|---|---|---|---|---|---|
| | IL6 | | | IFN-γ | | |
| Treatment details | Day 4 | Day 7 | Day 11 | Day 4 | Day 7 | Day 11 |
| TNBS intrarectally day 1 | 130 | | | 23.1 | | |
| (Sacrificed on day 4) | | | | | | |
| TNBS intrarectally day 1 + | | 117.4^{a} | 162.6^{b} | | 28.4^{b} | 34.43^{bc} |
| Control Vehicle (Distilled | | | | | | |
| water) orally/thrice daily from | | | | | | |
| day 4-10 | | | | | | |
| Saline intrarectally day 1 + | 106 | 116.4^{a} | 140^{ab} | 14.8 | 20.0^{a} | 26.06^{a} |
| Control Vehicle (Distilled | | | | | | |
| water) orally/thrice daily from | | | | | | |
| day 4-10 | | | | | | |
| TNBS intrarectally day 1 + | | 103.0^{a} | 140.1^{ab} | | 18.7^{a} | 29.96^{ab} |
| PB6 1.5 108 CFU/Kg orally/ | | | | | | |
| thrice daily from day 4-10 | | | | | | |
| TNBS intrarectally day 1 + | | 112.4^{a} | 144.6^{ab} | | 20.33^{a} | 34.23^{bc} |
| Lysophospholipid 10.3 mg/Kg | | | | | | |
| orally 2 times daily | | | | | | |
| from day 4-10 | | | | | | |
| TNBS intrarectally day | | 98.61^{a} | 126.7^{a} | | 17.9^{a} | 35.05^{bc} |
| 1 + Lysophospholipid + Omega- | | | | | | |
| 3 fatty acids + Carotenoids | | | | | | |
| combination 25.75 mg/ml | | | | | | |
| orally 2 times daily from day 4-10 | | | | | | |
| TNBS intrarectally day | | 103.9^{a} | 127.4^{a} | | 20.8^{a} | 40.23^{c} |
| 1 Mesacol 250 mg/Kg orally | | | | | | |
| Once daily from day 4-10 | | | | | | |

Values within the column are statistically significant (p < 0.05)

The major role pro-inflammatory cytokines (e.g. IL-1, IL-6, TNF-α) play in the pathogenesis of IBD is well established. A pro-inflammatory cytokine mediated inflammatory response is a cascade of gene products generally not evident in healthy individuals. Pro-inflammatory genes, including phospholipase A₂ (PLA₂), Cyclooxygenase (COX)-2 and inducible NO synthase code for enzymes that increase the production of platelet-activating factor and prostanoids, leukotreines, and NO. These small mediator molecules are up-regulated during inflammation. IL-1β and TNF act synergistically in stimulating the expression of these genes. Anti-inflammatory cytokines can mediate effects that down-regulate inflammatory responses (Wahl, 1992; Cassatella et al., 1993), thereby suppressing the intensity of the cascade. Consistent with the identified pro-inflammatory functions of NF-kB, the expression of many factors that are controlled by NF-kB, such as cytokines TNFα and IL-6 are also elevated in IBD (Martinez-Borra, 2002).

The changes in the anti-inflammatory cytokines such as IFN-γ and IL-6 are given in Table 15. The plasma level of IL 10 was higher in saline instilled rats than TNBS induced control vehicle but of insignificance in day 7 and 11. The levels of plasma IL 10 was significantly higher (p<0.05) in PB6 treated group in both day 7 and 11 than TNBS induced control vehicle. However in day 11, the standard drug Mesacol® treated group showed significantly highest (p<0.05) value of IL10 than TNBS induced control and saline instilled group.

TABLE 15
Anti inflammatory cytokines in plasma

| | Cytokine levels in Plasma (picogram/ml) | | | | | |
|---|---|---|---|---|---|---|
| | IL10 | | | TGF-β | | |
| Treatment details | Day 4 | Day 7 | Day 11 | Day 4 | Day 7 | Day 11 |
| TNBS intrarectally day 1 | 247 | | | 18042 ± 796a | | |
| (Sacrificed on day 4) | | | | | | |
| TNBS intrarectally day 1 + Control | | 177.3a | 250a | | 17845 ± 796a | 23853 ± 649a |
| Vehicle (Distilled water) orally/ | | | | | | |
| thrice daily from day 4-10 | | | | | | |
| Saline intrarectally day 1 + | 230 | 207.6ab | 290ab | 52676 ± 8459b | 33060 ± 3780b | 45851 ± 4724c |
| Control Vehicle (Distilled water) | | | | | | |
| orally/thrice daily from day 4-10 | | | | | | |
| TNBS intrarectally day 1 + PB6 | | 277.5b | 394bc | | 24143 ± 2173b | 34261 ± 2114b |
| 1.5 108 CFU/Kg orally/thrice | | | | | | |
| daily from day 4-10 | | | | | | |
| TNBS intrarectally day 1 + | | 219.8ab | 310ab | | 28679 ± 6805b | 29435 ± 4733a |
| Lysophospholipid 10.3 mg/Kg | | | | | | |
| orally 2 times daily from day 4-10 | | | | | | |
| TNBS intrarectally day | | 263.4ab | 399abc | | 34189 ± 8935b | 47995 ± 7378c |
| 1 + Lysophospholipid + Omega-3 | | | | | | |
| fatty acids + Carotenoids | | | | | | |
| combination 25.75 mg/ml orally 2 | | | | | | |
| times daily from day 4-10 | | | | | | |
| TNBS intrarectally day 1 Mesacol | | 217.9ab | 444c | | 17535 ± 2261a | 32542 ± 5294b |
| 250 mg/Kg orally once daily from | | | | | | |
| day 4-10 | | | | | | |

The plasma levels of TGF-β were significantly higher (p<0.01) in saline instilled rats than TNBS induced control vehicle in day 4, 7 and 11. The combination group and Lysophospholipid showed significantly higher levels (p<0.05) of plasma TGF-β. The PB5 treated groups showed significantly higher levels (p<0.05) in day 7 and maintained the levels there by in day 11 it was on par with TNBS induced control groups. The Mesacol® treated group showed lower levels of TGF-β than TNBS induced control vehicle in day 7 and significantly increased (p<0.05) levels in day11. Cytokines such as IL-10 and transforming growth factor TGF-β restrain the production of IL-1β and TNF-α. Several studies have shown that elevated levels of TNF-α in particular, feature strongly in IBD (1, 2). Therapeutic agents are being developed to limit or neutralize the action of agents involved in the inflammatory process.

### Role of Lysophospholipids in the Inflammatory Pathway

The lyso-pl components of the drug have the ability to regulate the expression, formation, and provocation of PLA2, thus controlling the synthesis and/or stimulation of lipid mediators, including eicosanoids and cytokines. Lyso-pls prevent any further release of these inflammatory mediators by combining with free AA present at the site of inflammation, thus modifying the lipid membrane composition. The mechanism responsible for altering this composition is the deacylation/reacylation cycle (Lands cycle: Lands & Crawford, 1976). The levels of AA must be firmly regulated and maintained at low concentrations by cells due to their strong involvement in the production of eicosanoids. Unlike saturated fatty acids, AA does not generally enter cellular phospholipids via the de novo pathway but accesses it at a later stage, via direct acylation of pre-existing lyso-pl acceptors. The availability of lyso-pl acceptors, predominantly Lysophospholipids, is essential for this incorporation of AA into phospholipids (Chilton et al., 1996). The incorporation of AA into Lysophospholipids involves the action of CoA-dependent acyltransferases. Subsequently, AA is transferred to other lyso-pls, particularly LPE, through CoA-independent transacylase (CoA-IT) activity (Chen & Wang, 2002). During cellular activation, increased AA reacylation results in the proportion of AA being incorporated back into phospholipids and only a minor portion is converted into proinflammatory eicosanoids (Lands, 2000). Thus, the reacylation pathway, catalyzed by lysophosphatidylcholine:acyl-CoA acyltransferases, could be a fundamental regulatory mechanism controlling the total quantity of prostaglandins and leukotrienes, by decreasing the absolute concentration of free AA through conversion of Lysophospholipids into arachidonate-containing phospholipids.

Lysophospholipids, the most bioactive component of the compound, demonstrates a high degree of specificity for cPLA2, which is principally the primary enzyme mediating AA mobilization in activated cells (Balsinde et al., 2000). By controlling the levels of cPLA2, the production of AA can be consistently maintained, in addition to an optimum balance of eicosanoid metabolites. Furthermore, the mucosal cell membranes can absorb lysophospholipids and PC, leading to its involvement in signaling processes that inhibit proinflammatory signalling associated with inflammation (Anes et al., 2003). Treede and colleagues (2007) molecularly described the anti-inflammatory properties of lysophospholipids. This phospholipid strongly inhibited TNF-α-induced NF-kB activation in Caco-2 cells, and clearly delayed the upregulation of pro-inflammatory genes ICAM-1, MCP-1, IL-8 and IP-10.

Additional plausible mechanisms have been proposed for the protective use of lysophospholipids against inflammation. Among them comprise a reduction in circulating levels of HMGB1 (Chen et al., 2005), decreased expression of tissue factor by monocytes/macrophages (Engelmann et al., 1999), enhanced bacterial killing by neutrophils, increased bacterial clearance (Yan et al., 2004), and the increased activation of eNOS by upregulating transcription and/or decreasing mRNA degradation (Zembowicz et al., 1995; Cieslik et al., 1998). The therapeutic effects could also be attributed to the proposed conception that Lysophospholipids is deficient in intestinal disease, namely UC. Analysis of rectoscopically acquired mucus aliquots exhibited a significantly diminished content of PC and lysophospholipids in patients with UC compared with controls or patients with CD (Ehehalt et al., 2004). As aforementioned, AA progresses at the expense of essential fatty acids. Thus, the decrease in Lysophospholipids may be associated with the modifications of other lipids, or indeed of PC. Phospholipids encircle the entire gastrointestinal tract, consisting mainly of PC and Lysophospholipids (Bernhard et al., 1995). The intestinal mucosal cells may be protected against injurious luminal products by a hydrophobic barrier formed by these phospholipids.

Numerous classes of Lysophospholipids exist that have shown beneficial effects against inflammation. Natural Lysophospholipids afforded protection against endotoxaemic animals (Murch et al., 2006). Synthetic lysophospholipids dose-dependently reduced organ injury and dysfunction caused by gram-negative shock, and was associated with reducing circulating levels of IL-1β (Murch et al., 2006). Stearoyl lysophospholipids (s lysophospholipids) is a potential therapeutic agent to controlling systemic inflammation that inhibits a late appearing high-mobility group box 1 (HMGB-1) protein secretion from activated macrophages (Chen et al., 2005). S lysophospholipids provides further protection against lethal sepsis in experimental animals (Yan et al., 2004; Chen et al., 2005) and demonstrates a reduction in circulating levels of TNF-α and IL-1β. Yan and colleagues (2004) reported protective effects of lysophospholipids against septic shock attributable to the ability of lysophospholipids to intensify bacterial clearance, diminish neutrophil deactivation and decrease the levels of TNF-α and IL-1β. Finally, lysophospholipids has also demonstrated an ability to reduce tissue factor release generated by LPS from monocytes (Engelmann et al., 1999), which may be of benefit in inflammatory conditions.

### Role of Omega-3 Fatty Acids in the Inflammatory Path Way

The interaction between omega-3 fatty acids and AA on COX activity could also partially explain the reduction of inflammation. Omega-3 fatty acids compete with AA to produce less inflammatory and chemotactic derivatives. Consequently, the most critical mediators of inflammation, eicosanoids and cytokines, are hindered. The protection offered against mucosal damage correlates with the synthesis of anti-inflammatory derivates of 0o3 fatty acids, down-regulation of pro-inflammatory cytokines, and up-regulation of protective factors in the colon (Babcock et al., 2000). The capacity of monocytes to generate proinflammatory IL-1I and TNF-α is suppressed by ω3 fatty acids through the suppression of IL-1β mRNA and expression of COX2 mRNA that is induced by IL-1β mRNA. Additional immunomodulatory mechanisms posed for ω3 fatty acids in IBD include modifying cell signal transduction, intraluminal bacterial content, cell membrane fluidity and gene expression (Teitelbaum et al., 2001).

Experimental evidence from cell culture (Chu et al., 1999), rodent (Wallace et al., 2000) and human clinical (Belluzzi, 2002) studies support the robust immunomodulatory effects mediated through an apparent distortion of eicosanoid synthesis and eicosanoid-independent inhibitory effects on the pro-inflammatory cytokine IL-1. Geerling and colleagues (2000) revealed a significantly decreased proportion of AA in patients with CD following three weeks supplementation of ω3 fatty acids, which could result in the generation of eicosanoids with a lesser amount of pro-inflammatory activity. Thus, the ameliorating potential of these PUFAs towards intestinal inflammation is noteworthy. Data are insufficient to draw conclusions concerning the ability of ω3 PUFAs to solely suppress IBD (MacLean et al., 2005), however, many studies have concluded that a higher intake of ω3 PUFA might exercise anti-inflammatory effects (Belluzzi, 2002). Furthermore, a new area has surfaced for the use of ω3 fatty acids in potentiating the effects of drugs or decreasing their toxicity (Clarke et al., 2001; Simopoulos, 2002).

### Role of Carotenoids in the Inflammatory Path Way

An exceptionally enhanced quantity of unsaturated double bonds may cause further oxidative damage in tissue that is previously depleted of antioxidants (Belluzi et al., 1996). This can effectively be prevented by administration of antioxidant compounds. The scavenging abilities of carotenoids for oxidants, oxygen radicals and nitric oxide, surmount the lipid peroxidation by-products and cell injury associated with long-chain PUFA (Nieto et al., 1998), thus providing protection to cells, tissues and plasma lipoproteins.

Antioxidants may further act in the inhibition of NFkB activation, and the subsequent reduction in the synthesis of pro-inflammatory mediators and adhesion molecules. Evidence from experimental colitis demonstrated their ability to prevent tissue damage (Choudhary et al., 2001). Thus, maintaining adequate antioxidant status may enhance the attenuation of cellular injury.

### Role of PB6 in the Inflammatory Path Way

Bacillus subtilis PB6, a natural probiotic, was found to secrete surfactins (cyclic lipopeptides) which have antibacterial, antimicrobial, antiviral, anti-inflammatory and antitumor potential. The surfactins secreted by PB6 inhibit phospholipase A2, a rate limiting enzyme involved in the arachidonic acid associated inflammatory pathway. Diverse of these have been found to be strong mediators of intestinal inflammation (Wallace and Ma, 2001). Cytosolic phospholipase A2 (cPLA2) requires phosphorylation by mitogen-activated protein kinase (MAPK) to be fully activated. During the inflammatory cascade, P38 MAPK is activated by its phosphorylation which in turn activates NF-kB which play a vital role in cytokine gene expression (TNF-α, IL-1β, IL-6, IL-10, TGF-β and IFN-γ) (Treisman, R.1996). The inhibition of cPLA2 could down regulate the inflammatory response by regulating the eicosanoid and cytokine production pathways.

In conclusion, the present result demonstrated that PB6 exerts a beneficial effect in the TNBS induced colitis in rats. As a possible mechanism, Bacillus subtilis PB6 could regulate the inflammation by down regulating the PLA2-p38 MAPK-NF-Kb pathway as reflected by inhibition of synthesis of pro-inflammatory cytokines and elevation in the level of anti-inflammatory cytokine and thereby re-establishing the cytokines balance in inflammation.

### CONCLUSION

The rationale for conducting this study was to investigate the efficacy of lysophospholipids, PB6 and a combination drug of lysophospholipids, omega-3 fatty acids and carotenoids at different concentrations on the amelioration of inflammation in the TNBS-rat model. The combination of lysophospholipids, omega-3 fatty acids and carotenoids at a lower concentration (21.3 mg/kg) was significantly effective in alleviating the symptoms associated with TNBS induced colitis in rats. The combination of lysophospholipids, omega-3 fatty acids and carotenoids at a lower concentration (21.3 mg/kg) was found to decrease the colon wet weight and colon morphological score significantly (p<0.01). Empirical evidence indicates that IBD activity is strongly correlated with pro and anti-inflammatory cytokine and our biochemical results also robustly signified this. The combination of lysophospholipids, omega-3 fatty acids and carotenoids at a lower concentration (21.3 mg/kg) significantly reduced (p<0.05) the pro inflammatory cytokines IL-1β and IL6 in day 11 and IFN-γ in day 7 than TNBS control. The combination of lysophospholipids, omega-3 fatty acids and carotenoids at a lower concentration (21.3 mg/kg) increased an anti-inflammatory cytokine ILIO and significantly increased (p<0.05) TGF-β in day 7 and 11. PB6 treated group was found to decrease the colon wet weight and colon morphological score significantly (p<0.01), and significantly reduced (p<0.05) the pro inflammatory cytokines TNF-α and IFN-γ in day 11 and IL-1β and IFN-γ IL6 in day 11 and in day 7 than TNBS control. Empirical evidence indicated that IBD activity is strongly correlated with pro and anti-inflammatory cytokines. Our biochemical results also robustly signified this. The possible way for the better activity of combination of lysophospholipids, omega-3 fatty acids and carotenoids at a lower concentration (21.3 mg/kg) in ameliorating the inflammation in colitis could be through restoring the Th1-Th2 cytokines balance by inhibiting the PLA2-MAPKinase pathway.

### REFERENCES

1. Anes E., Kuhnel M., Bos E. 2003. Selected lipids can activate phagosome actin assembly and maturation leading to the killing of pathogenic mycobacteria. Nat Cell Biol. 5; 793-802.
2. Asaoka Y., Oka M., Yoshida K., Sasaki Y., & Nishizuka Y. 1992. Role of Lysophosphatidylcholine in T-Lymphocyte Activation: Involvement of Phospholipase A2 in Signal Transduction through Protein Kinase C Proc. Natl. Acad. Sci. USA. 89(14);6447-6451.
3. Babcock T., Helton W. S & Espat N. J. 2000. Eicosapentanoic acid (OMEGA-3 FATTY ACIDS): an anti-inflammatory ω-3 fat with potential clinical applications. Nutrition 16; 1116-1118.
4. Baidoo L & Lichtenstein G. R. 2005. What next after infliximab? Am J Gastroenterol. 100; 80-83.
5. Balsinde J., Balboa M. A., Yedgar S & Dennis E. A. 2000. Group V phospholipase A₂-mediated oleic acid mobilization in lipopolysaccharide-stimulated P388D₁ macrophages. J. Biol. Chem. 275; 4783-4786.
6. Bamias G., Nyce M. R., De La Rue S. A., & Cominelli C. 2005. New concepts in the pathophysiology of inflammatory bowel disease, Ann Intern Med .143; 895-904.
7. Bell S J & Kamm M. A. 2000. Antibodies to tumor necrosis factor alpha as treatment for Crohn's disease. Lancet. 355;858-860
8. Belluzzi A. 2002. N-3 fatty acids for the treatment of inflammatory bowel diseases. Proc Nutr Soc. 61;391-395.
9. Belluzzi A., Brignola C, Campieri M, Pera A, Boschi S. & Miglioli, M. (1996) Effect of an Enteric-Coated Fish-Oil PrOmega-3 fatty acidsration on Relapses in Crohn's Disease N. Engl. J. Med. 334, 1557-1560.
10. Bernhard W., Postle A. D., Linck M., Sewing K. F. 1995.Composition of phospholipid classes and phosphatidylcholine molecular species of gastric mucosa and mucus. Biochim Biophys Acta. 1255;99-104.
11. Binder V. 2004. Epidemiology of IBD during the twentieth century: an integrated view. Best Pract Res Clin Gastroenterol. 18; 463-79.
12. Boughton-Smith N. K., Wallace J. L & Whittle B. J. R. 1988b. Relationship between arachidonic acid metabolism, myeloperoxidase activity and leukocyte infiltration in a rat model of inflammatory bowel disease. Agents Actions. 25; 115-123.
13. Boughton-Smith N. K., Wallace J. L., Morris G. P & Whittle B. J. R. 1988a. The effect of anti-inflammatory drugs on eicosanoid formation in a chronic model of inflammatory bowel disease in the rat. Br. J. Pharmacol. 94; 65-72.
14. Bouma G., and Strober W. 2003. The immunological and genetic basis of inflammatory bowel disease. Nat. Rev. Immunol. 3::521-533.
15. Chen G., Li J., Qiang X., Czura C. J., Ochani M., Ochani K., Ulloa L., Yang H., Tracey K. J., Wang P., Sama A. E., Wang H.2005. Suppression of HMGB-1 release by stearoyl lysophosphatidylcholine:an additional mechanism for its therapeutic effects in experimental sepsis. J Lipid Res. 46;623-627.
16. Chen M and Wang J.2002. Initiator caspases in apoptosis signaling pathways. Apoptosis 7; pp.313-319
17. Chilton F. H., Fonteh A. N., Surette M., Triggiani M and Winkler J. D. 1996. Control of arachidonate levels within inflammatory cells, Biochim. Biophys. Acta. 1299; pp. 1-15
18. Choudhary S., Keshavarzian A., & Yong S. 2001. Novel antioxidants zolimid and AEOL11201 ameliorate colitis in rats. Dig Dis Sci. 46; 2222-30.
19. Chu A. J., Walton M. A., Prasad J. K & Seto A. 1999. Blockade by polyunsaturated n-3 fatty acids of endotoxin-induced monocytic tissue factor activation is mediated by the depressed receptor expression in THP-1 cells. Journal of Surgical Research. 87;217-224.
20. Cohen R. D., Larson L. R., Roth J. M., Becker R. V & Mummert L. L. 2000. The cost of hospitalization in Crohn's disease. Am J Gastroenterol. 95; 524-530.
21. Ehehalt R., Wagenblast J& Erben G. 2004. Phosphatidylcholine and lysophosphatidylcholine in intestinal mucus of ulcerative colitis patients. A quantitative approach by electrospray-tandem mass spectroscopy. Scand J Gastroent. 39;737-42.
22. Fuss I. J. 2004. Nonclassical CD1d-restricted NK T cells that produce IL-13 characterize an atypical Th2 response in ulcerative colitis. J. Clin. Invest. 113:1490-1497.
23. Geerling B. J., Badart-Smook A & van Deursen C. 2000. Nutritional supplementation with N-3 fatty acids and antioxidants in patients with Crohn's disease in remission effects on antioxidant status and fatty acid profile. Inflamm Bowel Dis .6 (2); 77-84.
24. Hendrickson B. A., Gokhale R & Cho J. H. 2002. Clinical aspects and pathophysiology of inflammatory bowel disease. Clin. Microbiol. Rev. 15 (1);79-94.
25. Hugot J. P., Chamaillard M., Zouali H., Lesage S., Cezard J. P., Belaiche J., Almer S., Tysk C., O'Morain C. A., Gassull M. 2001. Association of NOD2 leucine-rich repeat variants with susceptibility to Crohn's disease. Nature. 411; 599-603.
26. Johnson P. D & Besselsen D. G. 2002.Practical Aspects of Experimental Design in Animal Research. ILAR Journal. 43 (4); 202-206.
27. Lands W. E & Crawford G. C. 1976. Enzymes of membrane phospholipid metabolism in animals. Synthesis of an unsaturated fatty acid analogue (18-(4′-azido-2′-hydroxybenzoylamino)-oleic acid) and its interaction with lysophosphatidylcholine: acyl-CoA-O-acyltransferase. The Journal of Lipid Research. 39; 1118-1126.
28. Lands W. E. M. 2000. Stories about acyl chains. Biochim. Biophys. Acta. 1483; 1-14
29. Lapidus A., Bernell O., Hellers G & Lofberg R. 1998. Clinical course of colorectal Crohn's disease: a 35-year follow-up study of 507 patients. Gastroenterology. 114; 1151-1160.
30. Lesage S., Zouali H., Cezard J. P., Colombel J. F., Belaiche J., Almer S., Tysk C., O'Morain C., Gassull M., Binder V. 2002. CARD15/NOD2 mutational analysis and genotype-phenotype correlation in 612 patients with inflammatory bowel disease. A J Hum Genet. 70; 845-57.
31. Lichtenstein G. R. 2001. Approach to corticosteroid-dependent and corticosteroid-refractory Crohn's disease. Inflamm Bowel Dis. 7(1); S23-S29.
32. MacLean C. H., Mojica W. A., Newberry S. J., Pencharz J., Garland R. H., Tu W., Hilton L. G., Gralnek I. M., & Rhodes S. 2005.Systematic review of the effects of n-3 fatty acids in inflammatory bowel disease. Am J Clin Nutr. 82; 611-19.
33. Maerten P., Shen C., Colpaert S., Liu Z., Bullens D. A & Van Assche G. 2004. Involvement of interleukin 18 in Crohn's disease: evidence from in vitro analysis of human gut inflammatory cells and from experimental colitis models. Clin Exp Immunol. 135 (2); 310-317.
34. Mannon P. J. 2004.Anti-interleukin-12 antibody for active Crohn's disease. N. Engl. J. Med. 351::2069-2079.
35. Martinez-Borra J., Lopez-Larrea C., Gonzalez S., Fuentes D., Dieguez A., Deschamps E. M., Perez-Pariente J. M., Lopez- Vazquez A., De Francisco R & Rodrigo L. 2002. High serum tumor necrosis factor-alpha levels are associated with lack of response to infliximab in fistulizing Crohn's disease. Am J Gastroenterol.97; 2350-2356.
36. Miller D. H., Khan O. A., Sheremata W. A., Blumhardt L. D., Rice G. P. A., Libonati M. A., Willmer-Hulme A. J., Dalton C. M., Miszkiel K. A., O'Connor P. W. 2003. A controlled trial of natalizumab for relapsing multiple sclerosis. New Eng. J. Med. 348, 15-23.
37. Monteleone G. 1997. Interleukin 12 is expressed and actively released by Crohn's disease intestinal lamina propria mononuclear cells. Gastroenterology. 112:1169-1178
38. Morris G. P., Beck P. L., Herridge M. S., Depew W. T., Szewczuk M. R., Wallace J. L.1989. Hapten-induced model of chronic inflammation and ulceration in the rat colon. Gastroenterology. 96;795-803.
39. Murch O., Collin M., Sepodes B., Foster S. J., Mota-Filip H & Thiemermann C. 2006. Lysophosphatidylcholine reduces the organ injury and dysfunction in rodent models of Gram-negative and Gram-positive shock. British Journal of Pharmacology. 148; 769-777.
40. Mwantembe O., Gaillard M. C., Barkhuizen M., Pillay V., Berry S. D., Dewar J. B., Song E. 2001. Ethnic differences in allelic associations of the interleukin-1 gene cluster in South African patients with inflammatory bowel disease (IBD) and in control individuals. Immunogenetics. 52; 249-254.
41. Nieto N., Fema´ndez M.I., Torres M. I., Ri´os A., Sua´rez M. D., Gil A. 1998. Dietary n-3 and n-6 long-chain polyunsaturated fatty acids affect cellular antioxidant defense system in rats with experimental ulcerative colitis induced by trinitrobenzenesulfonic acid. Dig Dis Sci 43(12):2676 -2687.
42. Odes S., Vardi H., Friger M., Wolters F & Russel M. G. 2006. Cost Analysis and Cost Determinants in a European Inflammatory Bowel Disease Inception Cohort With 10 Years of Follow-up Evaluation. Gastroenterology. 131(6); 2029.
43. Ogura Y., Inohara N., Benito A., Chen F. F., Yamaoka S & Nunez G. 2001.Nod2, a Nod1/Apaf-1 family member that is restricted to monocytes and activates NFkappaB. J Biol Chem. 276; 4812-18.
44. Podolsky D. K. 2002. The current future understanding of inflammatory bowel disease. Best Pract Res Clin Gastroenterol. 16; 933-943.
45. Rolandelli R. H., Saul S. H., Settle R. G., Jacobs D. O., Trerotola S. O., Rombeau J. L. 1988. Comparison of parenteral nutrition and enteral feeding with pectin in experimental colitis in the rat. Am J Clin Nutr.47;715-721
46. Sandbom W. J & Targan S. R. 2002. Biologic therapy of inflammatory bowel disease. Gastroenterology. 122; 1592.
47. Sands B E. 2000. Therapy of inflammatory bowel disease. Gastroenterology. 118; S68-S82.
48. Sawczenko A & Sandhu B. K. 2003. Presenting features of inflammatory bowel disease in Great Britain and Ireland. Archives of Disease in Childhood. 88;995-1000
49. Schmidt C. 2005.Expression of interleukin-12-related cytokine transcripts in inflammatory bowel disease: elevated interleukin-23p19 and interleukin-27p28 in Crohn's disease but not in ulcerative colitis. Inflamm. Bowel Dis. 11:16-23.
50. Shanahan F. 2002. Crohn's disease. Lancet. 359;62-9.
51. Simopoulos A. P. 2002. Omega-3 Fatty Acids in Inflammation and Autoimmune Diseases. Journal of the American College of Nutrition. 21(6), 495-505.
52. Siviter R., Lancashire J & Choonara 1. 2007. Mesalazine induced drug toxicity may mimic symptoms in Crohn's disease. Paediatric and Perinatal Drug Therapy; 8 (2)
53. Stefan Fichtner-Feigl., Ivan J. Fuss., Jan C. Preiss., Warren Strober and Atsushi Kitani. 2005. Treatment of Murine Th1- And Th2-Mediated Inflammatory Bowel Disease with Nf-κB Decoy oligonucleotides. J. Clin. Invest. 115:3057-3071.
54. Strober W & Fuss I. J. 2006. Experimental models of mucosal inflammation. Adv Exp Med Biol. 579; 55-97
55. Teitelbaum J. E & Walker W. A. 2001. Review: the role of omega-3 fatty acids in intestinal inflammation. J Nutr Biochem. 12:21-32.
56. Treede I., Annika Braun., Richard Sparla., Mark Kühnel., Thomas Giese., Jerrold R., Turner., Elsa Anes., Hassan Kulaksiz., Joachim Füllekrug., Wolfgang Stremmel., Gareth Griffiths., and Robert Ehehalt. 2007. Anti-inflammatory effects of phosphatidylcholine. J. Biol. Chem. papers in press published online ahead of print Jul. 18, 2007, 10.
57. Treisman, R. 1996.Regulation of transcription by MAP kinase cascades. Current Opinion in Cell Biology, 8:205-215.
58. Uhlig H. H., and Powrie F. 2003.Dendritic cells and the intestinal bacterial flora: a role for localized mucosal immune responses. J. Clin. Invest. 112::648-651.
59. Villegas I., La Cassa C., Orjales .A & Alascon D. L. L. 2003. Effects of dosmalfate, a new cytoprotective agent, on acute and chronic trinitrobenzene sulphonic acid-induced colitis in rats. Eur. J. Pharmacol. 460; 209-218.
60. Wallace, J. L and L Ma. 2001. Inflammatory mediators in gastrointestinal defense and injury. Exp Biol Med, 226: 1003-1015.
61. Wallace F. A., Miles E. A & Calder P. C. 2000. Activation state alters the effect of dietary lipids on pro-inflammatory mediator production by murine macrophages. Cytokine. 12; 1374-1379.
62. Wallace J. L., Keenan C. M. 1990. An orally active inhibitor of leukotriene synthesis accelerates healing in a rat model of colitis. Am J Physiol.258:G527-G534.
63. Whittle B. J., Varga C., Pósa A., Molnár A., Collin M & Thiemermann C. 2006. Reduction of experimental colitis in the rat by inhibitors of glycogen synthase kinase-3^{β}B. J. Pharmal. 147.575-582
64. Woodruff T. M., Arumugam T. V., Shiels I. A., Newman M. L., Ross P. A. 2005. A potent and selective inhibitor of group Ia secretory phospholipase A₂ protects rats from TNBS-induced colitis. International Immunopharmacology. 5(5); 883-892.
65. Yan J. J., Jung J. S., Lee J. E., Lee J & Huh S. O. 2004. Therapeutic effects of lysophosphatidylcholine in experimental sepsis. Nat Med 10.161-167.
66. Zareie M., Singh P. K., Irvine E. J., Sherman P. M., McKay D. M., Perdue M. H. 2001. Monocyte/macrophage activation by normal bacteria and bacterial products. Implications for altered epithelial function in Crohn's disease. Am J Pathol. 158:1101-1109.
67. Zembowicz A., Jones S. L., & Wu K. K. 1995. Induction of cyclooxygenase-2 in human umbilical vein endothelial cells by lysophosphatidylcholine. J Clin Invest .96: 1688-1692.

### Example 4

### Effect of Two Dose Combination of Lysophospholipds, Essential Fatty Acid and Lutein in TNBS Induced Colitis Rat Model

Inflammatory bowel diseases (IBD) are identified as significant causes of gastrointestinal disease, characterized by severe inflammation within the intestinal tract, primarily due to Th1 cell mediated immunity with substantial incidence in the developed world (Russell, 2000). IBD epitomizes one of the pathological disorders posing the most compelling need for new therapeutic strategies. Pharmacological therapies, including glucocorticoids, immunosuppressants and aminosalicylates have proven difficult and complex (Sands, 2000). The efficacy of these drugs in managing inflammatory conditions has been overshadowed by substantial side effects, thus limiting the continuation of treatment. Furthermore, some patients are refractory even to the combined use of these agents (Baidoo&Lichtenstein, 2005; Lichtenstein, 2001), and surgical resection of the colon and ileostomy is frequently required (Lapidus et al., 1998).

The precise aetiology of the disease remains an enigma, but is likely to involve multifactor interactions among genetic factors, immunological factors and environmental triggers (Sandborn & Targan, 2002). Chromosome 16 in particular is a well-replicated linkage of CD and contains the CARD15 susceptibility gene that is involved in the activation of the pro-inflammatory transcription factor, NF-Kb (Ogura et al., 2001), via the interaction with MAP-Kinase and ERK1 (Hugot et al., 2001). CARD 15, contains three rare disease susceptibility alleles (Hugot et al., 2001), and at least one of these alleles is present in 30-40 percent of European patients (Lesage et al., 2002), compared with 14 percent of controls.

The most notable factor in the aetiology of the disease however, is the disregulation of mucosal inflammatory response to bacterial flora (Shanahan, 2002). Studies on patients and animal models of colitis suggest that an initial immunological challenge involving an abnormal host response to endogenous or environmental antigens or microbes causes tissue injury (Bamias et al., 2005; Hendrickson, 2002). The mucosal immune system normally controls the down regulation of the enhanced host defense system, thereby preventing damage to the tissue. In IBD, however, the loss or deterioration of the immunomodulating mechanisms provides the opportunity for the normally down-regulated responses to amplify uncontrollably into a chronic, inflammatory condition (Strober et al., 2002).

Rat model of TNBS colitis, commonly used model for human IBD, is characterized by exaggerated Th1 cell reponse associated with up regulation of proinflammatory cytokines and down regulation of regulatory cytokines. Current study was undertaken to highlight the functions of the primary inflammatory pathway, phospholipase A₂ (PLA₂), in the production of eicosanoid and cytokines, and their regulation and significance to IBD. It also further cited the potential inhibitory pathways and emphasized the grounds that render the inhibition of the PLA₂ pathway particularly important in the treatment of this disease. Finally, the rationale for investigating the effects of lysophospholipids, omega-3 fatty acids and lutein on an in-vivo model of intestinal inflammation was elucidated.

### Materials and Methods:

The source of mesalazine was Mesacol® 5-amino salicylic acid tablets obtained from Sun Pharmaceutical Industries. The source of 2,4,6-trinitrobenzene sulfonic acid was Sigma-Aldritch as picrylsulfonic acid solution, 5% (w/v). The source of lysophospholipids was Lysoprin™ from Kemin Industries, Inc. The source of omega-3 fatty acids was Marinol® Powder from Loder's Croklaan. The source of lutein was FloraGLO® brand lutein from Kemin Industries, Inc.

### Preparation and Administration

Mesacol® and TNBS solutions were prepared and administered as described in Example 3.

The combination treatment consisted of Lysoprin as a source of LPC, Marinol® powder as a source of EPA and FloraGLO® lutein as a source of lutein. Due to the unidentified dose-response relationship of this combination treatment a high and low dose were ascertained. The high and low doses were administered thrice daily beginning on day four, after the evidential effects of TNBS-induction, and continued up to and including day ten. The low dose consisted of 10.3 mg/kg Lysoprin, 10.3 mg/kg Marinol®, and 5.1 mg/kg of lutein. The high dose group contained 20.6 mg/kg Lysoprin, 20.6 mg/kg Marinol®, and 10.3 mg/kg of lutein. The components were blended well to accomplish high and low dose concentrations of 4.3 mg/ml and 2.15 mg/ml, respectively. The combination of these compounds is water insoluble, thus to maximize the drug potential, distilled water was used as a diluent to emulsify the lipids. The combinations were stirred with 20 ml of distilled water as an emulsion for 10 ml/kg body weight, and vigorously shaken. Doses were prepared daily thrice for four hours inter-dosing and used immediately. An 18G feeding tube affixed to a suitably adjusted syringe was utilized to employ 10 ml/kg of the drug. The dose of administration was arrived at based on the effective employment of the test item in a human intervention trial that utilized the item in the treatment of inflammation associated with asthma and rheumatoid arthritis (Kavitha et al., unpublished). The human conversion factor was utilized (US FDA) to establish the equivalent animal dose in mg/kg.

### Study Design:

The study complied with all relevant federal guidelines and institutional policies. Thirty six Male Wister rats, aged 10-12 weeks and weighing 180-200 g were obtained from the National Centre for Laboratory Animal Sciences, Hyderabad, India. Animals were housed for one week before the onset of the experiment and acclimatized under standard laboratory conditions at a constant temperature of 19 -25° C., with a relative humidity of 30-70%, and a 12-h light/dark cycle (Johnson & Besselsen, 2002). Each cage contained three animals (Donnelly, 1995), with readily available rat pellet food and filtered water ad libitum throughout the study, with the exception of the twelve hours before the initiation of the trial. Veterinary health checks were carried out during acclimatization; the animals were randomly categorized into six groups (1 to 6).

Group 1 (n=6) were induced with TNBS colitis and was sacrificed three days following instillation to determine the resulting degree of colonic damage. The administration of TNBS in untreated rats in group 2 (n=6) provided a drug-free TNBS-positive control group. Group 3 (n=6) received saline and served as vehicle controls to determine baseline levels of different parameters and for comparison with TNBS Groups 4 and 5 (n=6 each) were induced with TNBS-colitis and received a low and high dose combination of Lysoprin as a source foe LPC, Marinol® as a source of omega-3 fatty acids and FloraGLO® brand lutein as a source of lutein and Group 6 (n=6) was induced with TNBS and received a standard treatment drug Mesacol®. The mean weight variation between the groups was minimal and did not exceed ±20%. The use of identification numbers, cage cards, and animal marking allowed the recognition of animal groups. All experimental protocols described in this report were approved by the Institutional Animal Ethics Committee in accordance with the procedures specified by the Committee for the Purpose of Control and Supervision of Experiments on Animals, regarding animal care and handling. All modus operand was strictly followed the standard operating procedures of the laboratory and good laboratory practice was maintained at all times. The details of treatment groups, dosage and concentration given are detailed in Table 16.

TABLE 16
Details of treatment groups, dosage and concentration

| Dose Group | Dose | Concentration | | |
|---|---|---|---|---|
| 1. TNBS Baseline Group. | 4 | ml/kg | 25 | mg/ml |
| TNBS induced no treatment. | | | | |
| 2. TNBS Positive-Control Group. | 10 | ml/kg | — | |
| TNBS induced. Distilled water given orally as a control | | | | |
| vehicle thrice/day from day 4-10. | | | | |
| 3. TNBS Negative Group (Saline). | 10 | ml/kg | — | |
| Saline induced. Distilled water given orally as a control | | | | |
| vehicle thrice/day from day 4-10. | | | | |
| 4. Low Dose Combination Group. (LDC) | 21.5 | mg/kg | 2.15 | mg/ml |
| TNBS induced. A low dose combination of | (10 | ml/kg) | | |
| Lysophospholipids, Omega-3 Fatty Acids, Lutein given orally | | | | |
| thrice/day from day 4-10. | | | | |
| 5. High Dose Combination Group. (HDC) | 43 | mg/kg | 4.3 | mg/ml |
| TNBS induced. A high dose combination of | (10 | ml/kg) | | |
| Lysophospholipids, Omega-3 Fatty Acids, Lutein given orally | | | | |
| thrice/day from day 4-10. | | | | |
| 6. Standard Treatment Group (Mesacol ®) | 250 | mg/kg | 25 | mg/ml |
| TNBS induced. Mesacol ® given orally thrice/day from | (10 | ml/kg) | | |
| day 4-10. | | | | |

The animals were weighed after randomization, day 1, day4, day7 and day11. The weight gains were calculated with this data on day 4, day 7 and day11.

The processes of blood collection, determination of colonic inflammation, measurement of cytokine concentration in the blood, ELISA procedure, and statistical analysis were all carried out as described in Example 3.

### Results and Discussion

Clinical and Physiological Changes of Rats Induced with TNBS-Colitis

The administration of TNBS in 50% ethanol resulted in the development of mild diarrhea, rectal bleeding and increased mucus within hours of instillation. Anorexia, weight loss and reduced activity ensued, and were most progressive during the first three days. The hunchback appearance of the majority of animals for 48 hours post TNBS-induction, which is an indication of severe abdominal pain, continued for the TNBS positive control group throughout the study. The severe weight loss witnessed in animals induced with TNBS-colitis mirrors human development of IBD (Podolsky et al., 2002), with prominent features including increased resting energy expenditure, body weight loss and likelihood of anorexia, especially during acute stages (Sawczenko&Sandhu, 2003). Morris and colleagues (1989) demonstrated significant correlations between weight loss and anorexia with increases in the severity of colitis. The occurrence of diarrhea associated with all TNBS groups also corresponded with the literature on IBD (Bell & Kamm, 2000). Zareie and colleagues (2001) provided evidence that physiological epithelial ion transport and transepithelial electrical resistance were altered through the activation of macrophages by bacteria and their products. Animals treated with a low dose combination of LPC, EPA and lutein was markedly effective in reducing the degree of diarrhea among the rats with TNBS-induced colitis. The saline controls demonstrated normal bowel function, consistent weight and activity throughout the trial.

Animal Performance with Respect to Body Weight and Changes:

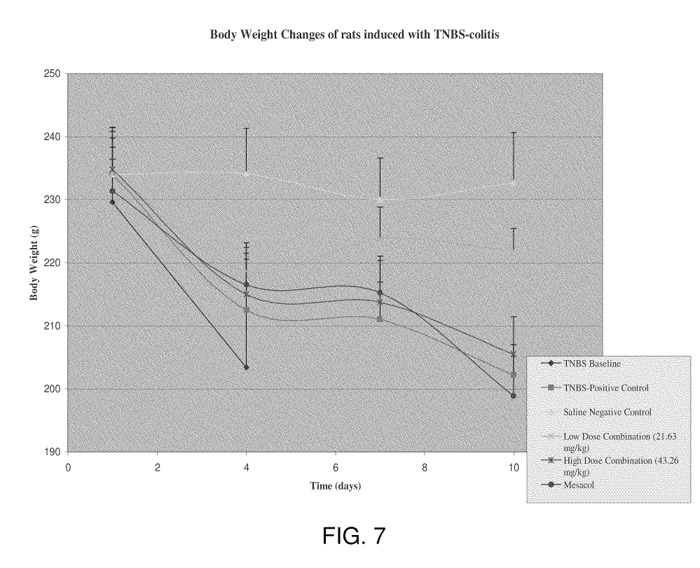

Prior to treatment, all animals administered with TNBS-colitis experienced significant weight loss compared with saline-control rats (p<0.05). The TNBS positive control group progressively lost weight with a mean loss of 31.83 g±6.65 (n =6), despite ad libitum feeding. In contrast, the weight of the saline-instilled group remained relatively consistent throughout the study, with a slight decrease after 10 days (1.33 g±1.82). All groups, except the group treated with a low dose combination of LPC, EPA and lutein revealed significant weight loss in comparison to the saline control group (p<0.01). Over the 11-day study period, highly significant changes in body weight were observed between groups (p<0.001) which is shown in FIG. 7. Although treatment with Mesacol® reduced the severity of inflammation to some degree, it was not as effective as the combination treatments at low doses. In fact, a significant difference in weight loss was observed between Mesacol® and the low dose combination of LPC, EPA and lutein group (20.88±6.41, p<0.05) following the initiation of treatment. Weight loss was so severe in the Mesacol® group that it resulted in two mortalities. This weight loss and accompanied diarrhoea emulates that of clinical trials in which diarrhea and weight loss is associated with mesalazine intake (Siviter et al., 2007). Additional medicines, including corticoids and immunosuppressants also produce a wide profile of substantial side effects (Sands, 2000).

Morphological and Macroscopic Changes in Colon of Rats Induced with TNBS Colitis

The degree of oedema determined by the colon weight and the degree of macroscopic lesions in the colon as colon score is given in Table 17 which significantly differed between groups (p<0.01). The combination of LPC, EPA and lutein at 21.63 mg/kg fed group demonstrated significantly (p<0.01) lower colon weights in comparison to the TNBS positive control group. The combination of LPC, EPA and lutein at 43.26 mg/kg fed group had higher colon weights that were comparable to the TNBS-control group. The low dose combination treatment of lysophospholipids, lutein and omega-3 fatty (21.63 mg/kg) resulted in a significant improvement in macroscopic scores compared with drug-free colitis control rats.

TABLE 17
Colon weight and colon score

| | Colon wet | Colon |
|---|---|---|
| | weight (g) | Score |
| TNBS intrarectally day 1 (Sacrificed on day 4) | 1.324^{c} | 4.200^{b} |
| TNBS intrarectally day 1 + Control Vehicle | 0.593^{b} | 3.500^{b} |
| (Distilled water) orally/thrice daily from day 4-10 | | |
| Saline Intrarectally day 1 + Control Vehicle | 0.222^{a} | 0.000^{a} |
| (Distilled water) orally/thrice daily from day 4-10 | | |
| TNBS intrarectally day 1 + Lysoprin + Marinol + | 0.225^{a} | 0.167^{a} |
| lutein at 21.5 mg/kg from day 4-10 orally/thrice | | |
| daily from day 4-10 | | |
| TNBS intrarectally day 1 + Lysoprin + Marinol + | 0.473^{ab} | 1.667^{a} |
| lutein at 43 mg/kg from day 4-10 orally/thrice | | |
| daily from day 4-10 | | |
| TNBS intrarectally day 1 + Mesacol 250 mg/Kg | 0.336^{ab} | 1.000^{a} |
| orally/once daily from day 4-10 | | |

Values within the column are statistically significant (p < 0.01)

The intracolonic administration of TNBS consistently produced severe colonic damage at three days post induction that continued until day 11. Macroscopic examination of inflamed lesion specimens of the two TNBS-colitis control groups demonstrated widespread damage, with obvious mucosal injury and inflammation, including hyperemia and swollen tissue. A large number of distal colon segments appeared thickened, exposing a deep reddish purple color with many hemorrhagic erosions and linear ulceration in the entire colonic wall in some cases. Damage scores were high in both groups with a mean score of 4.2 (±0.49) and 3.5 (±0.56) for TNBS baseline and TNBS positive control groups, respectively (Table 17). The inflammatory response is a collective intricate series of events induced by tissue damage. The multifaceted sequences of interactions that initiate these episodes comprise of various chemical mediators, albeit with inadequately understood relations. During the early phase of inflammation, an excessive quantity of interleukins and lipid-mediators act significantly in the pathogenesis of intestinal dysfunction. The membrane phospholipids release arachidonic acid (AA), resulting in the metabolism of pro-inflammatory prostaglandins (PGs) and leukotrienes. The bioactive mediators produced in response to inflammatory stimulus, such as prostanoids, cytokines and chemokines exhibit complex, pleitropic effects and interact with many cell types to magnify the inflammatory response. The deregulation of these processes may determine acute and chronic inflammatory disease.

The saline control groups showed no macroscopic lesions in the distal colon. The low dose combination of LPC, EPA and lutein at 21.63 mg/kg (Group 4) showed no visible inflammation or injury in the colonic tissue. Significant improvement, (p<0.01) in macroscopic scores were expressed by this group in comparison to colitis control rats. The high dose combination of LPC, EPA and lutein at 43.26 mg/kg (Group 5) showed reduced hyperemia, ulceration, and swelling in comparison to the colitis control. However, one sample demonstrated severe hyperemia and bowel wall thickening. The Mesacol® group (Group 6) (n=4/6) showed signs of amelioration with reduced localized ulceration, and a significant improvement (p<0.01) in macroscopic scoring in comparison to the TNBS-control group with no visible inflammation or injury in the colonic tissue.

Changes in the Cytokine Levels in Plasma of Rats Induced with TNBS Colitis

The changes in the pro inflammatory cytokines such as TNF-α, IL1-β and IL6 are given in Table 18. The plasma levels of TNF-a were significantly high (p<0.05) in TNBS induced control vehicle than the saline instilled group in day 7 and 11. Both the low dose combination and high dose combination given rats showed significantly lower (p<0.05) TNF-α levels in day 7 and day 11. However the levels of plasma TNF-α were least in lower dosage combination of LPC, EPA and lutein at 21.63 mg/kg and on par with the standard drug Mesacol®.

The plasma levels of IL 1beta were significantly high (p<0.05) in TNBS induced control vehicle than the saline instilled group in day 7 and 11. Both the low dose combination and high dose combination given rats showed significantly lower IL 1 beta levels. However the levels of plasma IL 1 beta was least in lower dosage combination of LPC, EPA and lutein at 21.63 mg/kg in day 11 and least in the standard drug Mesacol® at day 7.

The plasma levels of IL 6 were significantly high (p<0.05) in TNBS induced control vehicle than the saline instilled group in day 7 and 11. There was no improvement in the plasma levels of IL 6 in day 7 by both the lower and higher dosage combination groups and the standard drug Mesacol®. However the IL6 levels were significantly reduced in day 11 in the treatment group with lower dosage combination drugs. This showed that the drug dosage required a minimum of 7 days for its effective in improving the cytokine levels and elicit the inflammatory mechanism.

TABLE 18
Pro inflammatory cytokines in Plasma

| | Cytokine levels in Plasma (picogram/ml) | | | | | |
|---|---|---|---|---|---|---|
| | TNF-α | | | IL1-β | | |
| Treatment details | Day 4 | Day 7 | Day 11 | Day 4 | Day 7 | Day 11 |
| TNBS intrarectally day 1 (Sacrificed | 39±^{b} | | | 898±^{b} | | |
| on day 4) | | | | | | |
| TNBS intrarectally day 1 + Control | | 39±^{c} | 38±^{b} | | 854±^{b} | 811±^{c} |
| Vehicle (Distilled water) orally/thrice | | | | | | |
| daily from day 4-10 | | | | | | |
| Saline Intrarectally day 1 + Control | 28±^{a} | 30±^{ab} | 25±^{a} | 569±^{a} | 598±^{a} | 521±^{a} |
| Vehicle (Distilled water) orally/thrice | | | | | | |
| daily from day 4-10 | | | | | | |
| TNBS intrarectally day 1 + Lysoprin + | | 27±^{a} | 28±^{a} | | 697±^{ab} | 510±^{a} |
| Marinol + lutein at 21.5 mg/kg from | | | | | | |
| day 4-10 orally/thrice daily from day 4-10 | | | | | | |
| TNBS intrarectally day 1 + Lysoprin + | | 33±^{bc} | 30±^{a} | | 787±^{ab} | 562±^{ab} |
| Marinol + lutein at 43 mg/kg from | | | | | | |
| day 4-10 orally/thrice daily from day 4-10 | | | | | | |
| TNBS intrarectally day 1 + Mesacol | | 29±^{ab} | 28±^{a} | | 567±^{a} | 685±^{bc} |
| 250 mg/Kg orally/once daily from | | | | | | |
| day 4-10 | | | | | | |
| | Cytokine levels in Plasma (picogram/ml) | | | | | |
| | IL6 | | | IFN-γ | | |
| Treatment details | Day 4 | Day 7 | Day 11 | Day 4 | Day 7 | Day 11 |
| TNBS intrarectally day 1 (Sacrificed | 81±^{b} | | | 31 | | |
| on day 4) | | | | | | |
| TNBS intrarectally day 1 + Control | | 86±^{b} | 111±^{c} | | 37^{b} | 38^{bc} |
| Vehicle (Distilled water) orally/thrice | | | | | | |
| daily from day 4-10 | | | | | | |
| Saline Intrarectally day 1 + Control | 67±^{a} | 69±^{a} | 73±^{a} | 22 | 27^{a} | 28^{a} |
| Vehicle (Distilled water) orally/thrice | | | | | | |
| daily from day 4-10 | | | | | | |
| TNBS intrarectally day 1 + Lysoprin + | | 87±^{b} | 99±^{b} | | 36^{b} | 32^{ab} |
| Marinol + lutein at 21.5 mg/kg from | | | | | | |
| day 4-10 orally/thrice daily from day 4-10 | | | | | | |
| TNBS intrarectally day 1 + Lysoprin + | | 88±^{b} | 110±^{bc} | | 35^{b} | 34^{bc} |
| Marinol + lutein at 43 mg/kg from | | | | | | |
| day 4-10 orally/thrice daily from day 4-10 | | | | | | |
| TNBS intrarectally day 1 + Mesacol | | 104±^{c} | 104±^{bc} | | 37^{b} | 40^{c} |
| 250 mg/Kg orally/once daily from | | | | | | |
| day 4-10 | | | | | | |

Values within the column are statistically significant (p < 0.05)

The major role proinflammatory cytokines (e.g. IL-1, -6, TNF-α) play in the pathogenesis of IBD is well established. A pro-inflammatory cytokine mediated inflammatory response is a cascade of gene products generally not evident in healthy individuals. Pro-inflammatory genes, including phospholipase A₂ (PLA₂), cyclooxygenase (COX)-2, and inducible NO synthase code for enzymes that increase the production of platelet-activating factor and prostanoids, leukotreines, and NO. These small mediator molecules are up-regulated during inflammation. IL-1β and TNF act synergistically in stimulating the expression of these genes. Anti-inflammatory cytokines can mediate effects that down-regulate inflammatory responses (Wahl, 1992; Cassatella et al., 1993), thereby suppressing the intensity of the cascade.

The changes in the pro inflammatory cytokines such as IFN-γ and IL10 are given in Table 19.

TABLE 19
Anti inflammatory cytokines in Plasma

| | Cytokine levels in Plasma (picogram/ml) | | | | | |
|---|---|---|---|---|---|---|
| | IL10 | | | TGF | | |
| Treatment details | Day 4 | Day 7 | Day 11 | Day 4 | Day 7 | Day 11 |
| TNBS intrarectally day 1 (Sacrificed on day 4) | 232 | | | 31 | | |
| TNBS intrarectally day 1 + Control Vehicle | | 200^{a} | 217^{a} | | 37^{b} | 38^{bc} |
| (Distilled water) orally/thrice daily from day | | | | | | |
| 4 up to and including day 10 | | | | | | |
| Saline Intrarectally day 1 + Control Vehicle | 229 | 222^{ab} | 264^{ab} | 22 | 27^{a} | 28^{a} |
| (Distilled water) orally/thrice daily from day | | | | | | |
| 4 up to and including day 10 | | | | | | |
| TNBS intrarectally day 1 + Lysoprin + | | 242^{b} | 334^{c} | | 36^{b} | 32^{ab} |
| Marinol + lutein at 21.5 mg/kg from day 4-10 | | | | | | |
| orally/thrice daily from day 4 up to and | | | | | | |
| including day 10 | | | | | | |
| TNBS intrarectally day 1 + Lysoprin + | | 242^{ab} | 288^{bc} | | 35^{b} | 34^{bc} |
| Marinol + lutein at 43 mg/kg from day 4-10 | | | | | | |
| orally/thrice daily from day 4 up to and | | | | | | |
| including day 10 | | | | | | |
| TNBS intrarectally day 1 + Mesacol 250 mg/Kg | | 231^{ab} | 454^{d} | | 37^{b} | 40^{c} |
| orally/once daily from day 4 up to | | | | | | |
| and including day 10 | | | | | | |

The plasma level of IL 10 was higher in saline instilled rats than TNBS induced control vehicle but of insignificance in day 7 and 11. The levels of plasma IL 10 was significantly higher in lower dosage combination of LPC, EPA and lutein at 21.63 mg/kg in both day 7 and 11 than TNBS induced control vehicle. However in day 11, the standard drug Mesacol® treated group showed highest value of IL10.

The plasma levels of IFN-γ were significantly high (p<0.05) in TNBS induced control vehicle than the saline instilled group in day 7 and 11. Among the low dose and high dose combinations and Mesacol®, no improvement was seen when compared to TNBS induced Control vehicle in day 7. In day 11, the Mesacol® treated group showed significantly higher plasma IFN-γ levels, but the low dose combination and high dose combination given rats no improvement in IFN-γ levels.

Cytokines such as IL-10 and transforming growth factor (TGF)-b restrain the production of IL-10 and TNF-α. Several studies have shown that elevated levels of TNF-α in particular, feature strongly in IBD (1, 2). Therapeutic agents are being developed to limit or neutralize the action of agents involved in the inflammatory process.

### The Role of Lysophospholipids, Omega 3 Fatty Acids and Lutein in the Inflammatory Path Way:

### Lysophospholipids:

The lyso-pl components of the drug have the ability to regulate the expression, formation, and provocation of PLA2, thus controlling the synthesis and/or stimulation of lipid mediators, including eicosanoids and cytokines. Lyso-pls prevent any further release of these inflammatory mediators by combining with free AA present at the site of inflammation, thus modifying the lipid membrane composition. The mechanism responsible for altering this composition is the deacylation/reacylation cycle (Lands cycle: Lands and Crawford, 1976). The levels of AA must be firmly regulated and maintained at low concentrations by cells due to their strong involvement in the production of eicosanoids. Unlike saturated fatty acids, AA does not generally enter cellular phospholipids via the de novo pathway but accesses it at a later stage, via direct acylation of pre-existing lyso-pl acceptors. The availability of lyso-pl acceptors, predominantly LPC, is essential for this incorporation of AA into phospholipids (Chilton et al., 1996). The incorporation of AA into LPC involves the action of CoA-dependent acyltransferases. Subsequently, AA is transferred to other lyso-pls, particularly LPE, through CoA-independent transacylase (CoA-IT) activity (Chen & Wang, 2002). During cellular activation, increased AA reacylation results in the proportion of AA being incorporated back into phospholipids and only a minor portion is converted into proinflammatory eicosanoids (Lands, 2000). Thus, the reacylation pathway, catalyzed by lysophosphatidylcholine:acyl-CoA acyltransferases, could be a fundamental regulatory mechanism controlling the total quantity of prostaglandins and leukotrienes, by decreasing the absolute concentration of free AA through conversion of LPC into arachidonate-containing phospholipids.

LPC, the most bioactive component of the compound, demonstrates a high degree of specificity for cPLA2, which is principally the primary enzyme mediating AA mobilization in activated cells (Balsinde et al., 2000). By controlling the levels of cPLA2, the production of AA can be consistently maintained, in addition to an optimum balance of eicosanoid metabolites. Furthermore, the mucosal cell membranes can absorb LPC and PC, leading to its involvement in signalling processes that inhibit proinflammatory signalling associated with inflammation (Anes et al., 2003). Treede and colleagues (2007) molecularly described the anti-inflammatory properties of LPC. This phospholipid strongly inhibited TNF-α-induced NF-kB activation in Caco-2 cells, and clearly delayed the upregulation of proinflammatory genes ICAM-1, MCP-1, IL-8 and IP-10.

Additional plausible mechanisms have been proposed for the protective use of LPC against inflammation. Among them comprise a reduction in circulating levels of HMGB1 (Chen et al., 2005), decreased expression of tissue factor by monocytes/macrophages (Engelmann et al., 1999), enhanced bacterial killing by neutrophils, increased bacterial clearance (Yan et al., 2004), and the increased activation of eNOS by upregulating transcription and/or decreasing mRNA degradation (Zembowicz et al., 1995; Cieslik et al., 1998). The therapeutic effects could also be attributed to the proposed conception that LPC is deficient in intestinal disease, namely UC. Analysis of rectoscopically acquired mucus aliquots exhibited a significantly diminished content of PC and LPC in patients with UC compared with controls or patients with CD (Ehehalt et al., 2004). As aforementioned, AA progresses at the expense of essential fatty acids. Thus, the decrease in LPC may be associated with the modifications of other lipids, or indeed of PC. Phospholipids encircle the entire gastrointestinal tract, consisting mainly of PC and LPC (Bernhard et al., 1995). The intestinal mucosal cells may be protected against injurious luminal products by a hydrophobic barrier formed by these phospholipids.

Numerous classes of LPC exist that have shown beneficial effects against inflammation. Natural LPC afforded protection against endotoxaemic animals (Murch et al., 2006). Synthetic LPC dose-dependently reduced organ injury and dysfunction caused by gram-negative shock, and was associated with reducing circulating levels of IL-1β (Murch et al., 2006). Stearoyl LPC (sLPC) is a potential therapeutic agent to controlling systemic inflammation that inhibits a late appearing high-mobility group box 1 (HMGB-1) protein secretion from activated macrophages (Chen et al., 2005). sLPC provides further protection against lethal sepsis in experimental animals (Yan et al., 2004; Chen et al., 2005) and demonstrates a reduction in circulating levels of TNF-α and IL-1β. Yan and colleagues (2004) reported protective effects of LPC against septic shock attributable to the ability of LPC to intensify bacterial clearance, diminish neutrophil deactivation and decrease the levels of TNF-α and IL-1β. Finally, LPC has also demonstrated an ability to reduce tissue factor release generated by LPS from monocytes (Engelmann et al., 1999), which may be of benefit in inflammatory conditions.

### Omega-3 Fatty Acids:

The interaction between EPA and AA on COX activity could also partially explain the reduction of inflammation. EPA competes with AA to produce less inflammatory and chemotactic derivatives. Consequently, the most critical mediators of inflammation, eicosanoids and cytokines, are hindered. The protection offered against mucosal damage correlates with the synthesis of anti-inflammatory derivates of ω3 fatty acids, down-regulation of pro-inflammatory cytokines, and up-regulation of protective factors in the colon (Babock et al., 2000). The capacity of monocytes to generate proinflammatory IL-1β and TNF-α is suppressed by ω3 fatty acids through the suppression of IL-1β mRNA and expression of COX2 mRNA that is induced by IL-1β mRNA. Additional immunomodulatory mechanisms posed for ω3 fatty acids in IBD include modifying cell signal transduction, intraluminal bacterial content, cell membrane fluidity and gene expression (Teitelbaum et al., 2001).

Experimental evidence from cell culture (Chu et al., 1999), rodent (Wallace et al., 2000) and human clinical (Belluzzi, 2002) studies support the robust immunomodulatory effects mediated through an apparent distortion of eicosanoid synthesis and eicosanoid-independent inhibitory effects on the proinflammatory cytokine IL-1. Geerling and colleagues (2000) revealed a significantly decreased proportion of AA in patients with CD following three weeks supplementation of ω3 fatty acids, which could result in the generation of eicosanoids with a lesser amount of proinflammatory activity. Thus, the ameliorating potential of these PUFAs towards intestinal inflammation is noteworthy. Data are insufficient to draw conclusions concerning the ability of ω3 PUFAs to solely suppress IBD (MacLean et al., 2005), however, many studies have concluded that a higher intake of ω3 PUFA might exercise anti-inflammatory effects (Belluzzi, 2002). Furthermore, a new area has surfaced for the use of ω3 fatty acids in potentiating the effects of drugs or decreasing their toxicity (Clarke et al., 2001; Simopoulos, 2002).

### Lutein:

An exceptionally enhanced quantity of unsaturated double bonds may cause further oxidative damage in tissue that is previously depleted of antioxidants (Belluzi et al., 1996). This can effectively be prevented by administration of antioxidant compounds. The scavenging abilities of lutein for oxidants, oxygen radicals and nitric oxide, surmount the lipid peroxidation by-products and cell injury associated with long-chain PUFA (Nieto et a., 1998), thus providing protection to cells, tissues and plasma lipoproteins.

Antioxidants may further act in the inhibition of NFkB activation, and the subsequent reduction in the synthesis of proinflammatory mediators and adhesion molecules. Evidence from experimental colitis demonstrated their ability to prevent tissue damage (Choudhary et al., 2001). Thus, maintaining adequate antioxidant status may enhance the attenuation of cellular injury.

### CONCLUSION

The rationale for conducting this study was to investigate the efficacy of a combination drug of lysophospholipids, omega-3 fatty acids and lutein at different concentrations on the amelioration of inflammation in the TNBS-rat model. The combination of LPC, EPA and lutein at a lower concentration (21.3 mg/kg) is effective in alleviating the symptoms associated with TNBS induced colitis in rats. The combination of LPC, EPA and lutein at a lower concentration (21.3 mg/kg) is found to decrease the body weight loss, colon wet weight and colon morphological score significantly (p<0.05 and 0.01) and this effect is comparable to a standard drug, Mesacol®. Empirical evidence indicates that IBD activity is strongly correlated with pro- and anti-inflammatory cytokine and our biochemical results also robustly signified this, The combination of LPC, EPA and lutein at a lower concentration (21.3 mg/kg) is known to significantly reduce (p<0.05) the pro inflammatory cytokines (TNF-α, IL-1β and IL6) and increase an anti-inflammatory cytokine, IL10. The combination of LPC, EPA and lutein at a lower concentration (21.3 mg/kg) restores the Th1-Th2 cytokines balance by inhibiting the PLA2-MAPKinase pathway.

### Example 5

### Inflammatory Effect of Lysophosphatidyl Choline

Lysophosophatidylcholine (LPC) is generated by hydrolysis of phosphotidylcholine by phospholipase A2. With higher hydrophilicity and lower critical micelle concentration, LPC is considered as a superior biosurfactant to phosphatidylcholine. In addition to its emulsifying activity, several inflammatory diseases such as ischemia and asthma are linked with increased level of LPC (Huang et al., 2005), which can be explained as LPC increases permeability of cell barriers.

Nuclear Factor κB (NFκB) is a pleiotropic transcription factor which plays an important role in regulation of expression of multiple inducible genes including some key genes involved in inflammatory diseases (Brand et al., 1996). Activation of NFκB indicates proinflammatory effect and inhibition of NFκB indicates anti-inflammatory effect. Previous research reported that low concentrations (5 to 15 μmol/L) of LPC significantly increased NF-κB activity whereas higher concentration (50 μmol/L) of LPC inhibited the activity (Sugiyama, 1998). In this study, we used HepG₂-NFκB stable cell line to examine whether LPC plays proinflammatory or anti-inflammatory roles by regulating NFκB activity. Luciferase was used as a reporter gene.

### Materials and Methods

### Materials

PBS, DMSO, 0.05% trypsin solution (Gibco), TNF-α (BD), FBS, 0.4% Trypan Blue, 0.5% MTT, AlamarBlue, 1 mg/ml G418 (Merck), Lipofectamine 2000 (Invitrogen), Luciferase bifocal detection kit (Promega), Luciferase Steady Glo bifocal detection kit (Promega), DMEM media (Hyclone), RPI1640 media (Hyclone), Opti-MEM media (Invitrogen).

Pure lysophosphatidylcholine was purchased from Sigma. HepG₂ cell, HepG₂/NFκB-Luc cell lines were constructed by Chinese Academy of Science. Lysoprin and Bolec were obtained from warehouse of Kemin Agrifoods China. Emulfluid was sent from Kemin Agrifoods Europa. The LPC levels were 99%, 2.44%, 3.53% and 5.71% for LPC standard, Lysoprin, Bolec and Emulfluid, respectively. Since LPC molecules have a wide range of different molar masses and the composition of different LPC molecules in LPC samples are not clear, we determined to take the average of 550 dalton and adjusted the final molar concentrations as used in the assays using solution of 50% PBS+50% DMSO.

### Cytotoxicity Test

An overnight culture of HepG2/NFκB-Luc cells were trypsinized and transferred to 96-well microtiter plate, with 1.5×10⁴ cells/well. The 96-well microtiter plates were incubated for 24 hours at 37° C. with 5% CO₂. Then different concentrations of LPC samples were added into plate wells together with AlamarBlue. The cells were incubated for 4 hours at 37° C. with 5% CO₂ before the OD570 and OD600 readings were measured. Culture medium with no LPC added was used as a control. The cytotoxicity was reflected by the amount of AlamarBlue being reduced (reduction rate (RR)) as determined by the following formula:

RR %={[(εoxλ2)(Aλ1)−(εoxλ1)(Aλ2)]}/{[(εredλ1)(A′λ2)−(εredλ2)(A′λ1)]}×100%

where, εredλ1=155677 (absorption coefficient of reduced AlamarBlue at 570 nm)
Εredλ2=14652 (absorption coefficient of reduced AlamarBlue at 600 nm)
εoxλ1=80586 (absorption coefficient of oxidized AlamarBlue at 570 nm)
εoxλ2=117216 (absorption coefficient of oxidized AlamarBlue at 600 nm)
A′λ1=OD570 of the blank control (wells with AlamarBlue but no cells)
A′λ2=OD600 of the blank control (wells with AlamarBlue but no cells).

### Inflammation Test Without TNF-α

An overnight culture of HepG2/NFκB-Luc cells were trypsinized and transferred to 96-well microtiter plate, with 1.5×10⁴ cells/well. The 96-well microtiter plates were incubated for 24 hours at 37° C. with 5% CO₂. The supernatant was removed and 100 μl of cell culture medium containing different LPC levels (5, 15 and 50 μM) were added into each well and the microtiter plate was incubated for 6 hours at 37° C. with 5% CO₂ Each treatment was done in triplicates. After incubation, the supernatant was removed, and 100 μl of luciferase substrate was added into each well and incubated for 5 minutes. Then 80 μl of the solution was transferred to a clean microtiter plate to determine the florescence unit by Steady-Glo® Luciferase Assay System. The LPC-treated cells without substrate added were used as control.

Inflammation Test with TNF-α

The inflammation test with TNF-α was performed in the same way as the inflammation test without TNF-α, except that 30 ng/ml of TNF-α was added into each well at the same time when LPC samples were added.

### Results

### Cytotoxicity Test

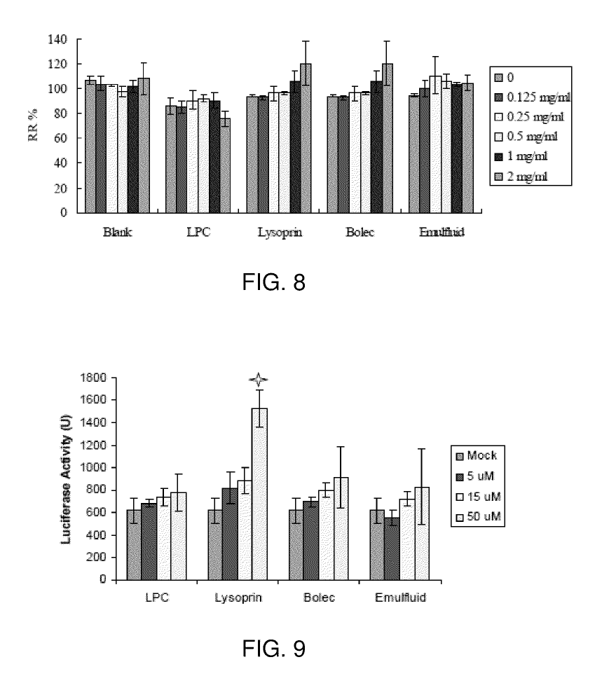

The result of cytotoxicity test was summarized in FIG. 8. The samples used were normalized by the LPC content. The cells were cultured with LPC for 4 hours before the reduced AlamarBlue was determined. If there is no cytotoxicity, then 100% of RR would be expected. In FIG. 8, it is clear that no cytotoxicity was observed for the blank control. Certain inhibition was observed for pure LPC when 2 mg/ml (˜3636 μM) of LPC was used, which is consistent with previous report. Contrary to expectation, at high level (2 mg/ml) Lysoprin and Bolec somehow showed growth promotion instead of cytotoxicity as observed for pure LPC, suggesting that some molecule(s) in Lysoprin and Bolec has growth promotion activity. No cytotoxicity or growth promotion was found for Emulfluid at all the concentrations tested. Considering LPC has slight cytotoxicity at 2 mg/ml, no inhibition was found in Emulfluid at this concentration of LPC suggesting that the growth promotion molecule(s) may also exist in Emulfluid.

### Inflammation Test Without TNF-α

HepG₂/NFκB-Luc is a stable cell line to measure the activity of the transcriptional factor NFκB which is actively involved in the activation of multiple genes involved in inflammation. The reporter gene is luciferase which can be easily determined by florescence measurement. Previously one paper reported that LPC has proinflammatory effect at 5-15 μM but anti-inflammatory effect at 50 μM in HUVEC cells. Moreover, this proinflammatory or anti-inflammatory effect was linked by NF-κB (Sugiyama et al, 1997). Therefore in this experiment 5 μM, 15 μM and 50 μM of LPC was used.

The result of inflammation test is presented in FIG. 9. Pure LPC demonstrated an inflammatory effect at all the three concentrations tested. However, no anti-inflammatory effect was observed at the high concentration. Instead, the inflammatory effect was more pronounced at higher concentration of LPC.

Bolec and Emulfluid showed similar patterns to that of pure LPC. The inflammatory effect became more pronounced when higher levels of LPC were used. Even at the highest concentration tested (50 μM of LPC), no statistical difference was observed for pure LPC, Bolec or Emulfluid compared to the mock treatment.

Lysoprin, in contrast, showed strong inflammatory effect at 50 μM of LPC. An apparent peak of luciferase activity was observed at 50 μM LPC, suggesting a very strong proinflammatory effect. This suggests that proinflammatory molecule(s) other than LPC exists in Lysoprin, but not in Bolec or Emulfluid.

Inflammation Test with TNF-α

The purpose of this experiment is to examine the anti-inflammatory effect of different sources of LPC. As a strong inflammatory factor, TNF-α activates the nuclear translocation of NF-κB and binds to the cis-elements of the inducible genes to activate transcription, and thus trigger the expression of the reporter gene, which is luciferase in our assay. Anti-inflammatory factor can alleviate the transactivation and lead to reduced luciferase activity.

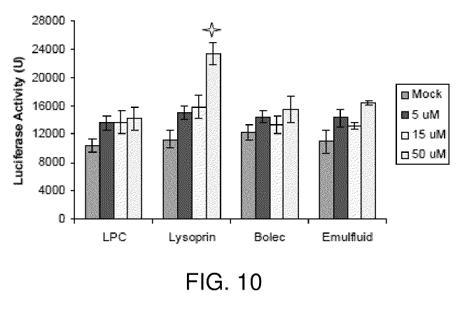

The result is presented in FIG. 10. No anti-inflammatory effect was observed for any of the treatments. Similar to what was seen in FIG. 9, LPC showed moderate proinflammatory effect, and both Bolec and Emulfluid showed the similar pattern as pure LPC. Lysoprin again demonstrated strong proinflammatory effect, suggesting an unidentified molecule(s) in Lysoprin has strong proinflammatory activity.

### DISCUSSION AND CONCLUSIONS

Previously it was reported that 50 μM of LPC showed anti-inflammatory effect in HUVEC/NFκB cells while lower concentrations (5 and 15 μM) of LPC had proinflammatory effect. Here we found that at all three concentrations tested (5, 15 and 50 μM), LPC demonstrated only proinflammatory but no anti-inflammatory effect. This discrepancy from the previous report may be explained by three reasons. First of all, different cell lines may have different sensitivities to LPC. It is possible that HepG₂ cell line is less sensitive so that higher concentration of LPC may be required to trigger anti-inflammatory effect. The second explanation is the complexity of LPC molecules. LPC is a mixture of different lysophosphatidylcholine molecules (e.g., α-palmitoyl-LPC, α-stearoyl-LPC, etc.), with different lengths of fatty acids and different levels of saturation of fatty acids in the C1 position. In our study, when the molar concentration was calculated, the average molar mass of 550 dalton was used, which very likely was inaccurate. Moreover, different compositions of LPC molecules with different lengths or saturation of fatty acids may play different roles in anti-inflammatory/proinflammatory pathways. Therefore simply normalizing LPC content did not take into consideration of the different compositions of different LPC molecules.

In both inflammation tests with or without TNF-α, Bolec and Emulfluid showed similar response as pure LPC, suggesting that the major proinflammatory effect in Bolec and Emulfluid attributes to LPC. However Lysoprin consistently showed strong proinflammatory effect, suggesting there exists proinflammatory molecule(s) other than LPC. The Lysoforte product in Kemin Agrifoods China generally contains ˜1% of LPC and is applied at 500 g of Lysoforte per ton of complete feed, which is around 10 μM if 550 dalton is used as molar mass. At 10 μM, Lysoprin already showed higher proinflammatory activity than that of Bolec and Emulfluid. Therefore it is recommended that Lysoprin not used for producing Lysoforte.

In summary we compared three sources of LPC (Lysoprin, Bolec and Emulfluid) in cytotoxicity and inflammation. Our results showed that none of the three sources of LPC had cytotoxicity when applied at very high level. In addition, Bolec and Emulfluid showed moderate proinflammatory effect, in a similar extent to pure LPC, but Lysoprin showed strong proinflammatory effect in NFκB pathways.

### REFERENCES

Brand K., Page S., Rogler G., Bartsch A., Brandl, R., Knuechel R., Page M., Kalschmidt C., Baeuerle P. A. and Neumeier D., 1996, Activated transcriptional factor nuclear factor-kappa B is present in the atherosclerotic lesion. J.Clin. Invest. 97:1715-1722
Huang F., Subbaniah, P. V., Holian, O., Zhang J., Johnson, A., Gertzberg N. and Lum H., 2005, Lysophosphatidylcholine increases endothelial permeability: role of PKCa and RhoA cross talk. Am J. Physiol. Lung Cell Mol Physiol. 289:176-185.
Sugiyama S., Kugiyama K., Ogata N., Doi H., Ota Y., Ohgushi M., Matsumura T., Oka Hi., Yasue H., 1998, Biphasic regulation of transcriptiona factor nuclear factor kB activity in human endotelial cells by lysophosphatidylcholine through protein kinase C-mediated pathway. Arterioscler Thromb Vasc Biol. 18:568-576

### Example 6

### Effect of Lysophospholipids, More Particular Lysophosphatidylcholine (LPC) on Asthma and Arthritis in Humans

Oxidative stress has been well described in asthma. Oxidative stress describes the damage that occurs when reactive oxygen species (ROS) overwhelm the antioxidant defenses of the host. In asthma, a variety of triggers, including allergens and viruses, lead to the recruitment and activation of airway inflammatory cells, which produce excess ROS, resulting in oxidative damage. Oxidative damage causes many detrimental effects on airway function, including airway smooth muscle contraction, airway hyper-responsiveness epithelial shedding and vascular exudation. Each of these effects contributes to the airway obstruction that is characteristic of asthma.

Host defense against ROS is provided by a range of antioxidants. The respiratory tract lining fluid (RTLF) forms an interface between the respiratory tract epithelial cells and the external environment, and thus forms the first line of defense against oxidative damage. The RTLF contains a range of antioxidants, including antioxidant enzymes (superoxide dismutase, glutathione peroxidase, catalase), metal-binding proteins (lactoferrin, transferrin, ceruloplasmin), and a range of low molecular weight antioxidants such as ascorbate, urate and glutathione. Carotenoids are another group of low molecular weight antioxidants that are likely to play an important role as antioxidants in the respiratory tract.

Epidemiological evidence has indicated that carotenoids may be important to respiratory health. There is a growing body of evidence linking carotenoid-rich foods to respiratory endpoints. Fresh fruit intake has been shown to be inversely associated with wheeze, chronic lung disease onset and was positively associated with % predicted forced expiratory volume in 1 second (% FEV1). Total fruit and vegetable intake has been inversely related to asthma prevalence but not related to % FEV1 or airway obstruction. Tomato products (uice, sauce, pizza) have been shown to be protective against asthma onset in a large longitudinal study, but no relationship was seen in a smaller case-control study. Vegetables have been shown to be protective against chronic bronchitis, bronchial asthma and wheeze. Studies examining dietary intake of specific carotenoids have focused on β-carotene. Several studies have shown a protective effect of β-carotene intake on one or more respiratory endpoints, while others have shown no effect. One recent study examined intake of a range of key carotenoids and found intake of lutein/zeaxanthin was positively associated with lung function. These data suggest carotenoids may play an important role in respiratory health and reinforce the need to characterize the range of key carotenoids when studying antioxidant defenses in asthma.

To date, our understanding of the role of carotenoids in asthma is limited by the absence of data on airway carotenoid levels.

The relationship between airway and circulating bio-markers of antioxidant defense is unknown. In fact, a recent report has indicated that for some biomarkers, blood levels do not represent what is happening at the airway surface (Improved antioxidant and fatty acid status of patients with cystic fibrosis after antioxidant supplementation is linked to improved lung function; Lisa G. Wood et al; Am J Clin Nutr 2003; 77:150-9). Thus analysis of oxidative stress and antioxidants in asthma should examine both circulating and airway biomarkers. The study was done to characterize circulating and airway levels of carotenoids in asthma compared to healthy controls, in relation to dietary intake. The study also tested whether airway lutein levels in healthy controls can be improved using oral supplements.

It was found that there were reduced levels of total carotenoids and all individual carotenoids measured, including lycopene, lutein, β-cryptoxanthin, α-carotene and β-carotene, in the whole blood of asthmatics versus healthy controls. It is uncertain why this deficiency was only observed in whole blood and not plasma or induced sputum. It is possible that this is related to the role of carotenoids in protecting erythrocyte membranes from oxidation. This is the first study to report levels of carotenoids in induced sputum. Induced sputum carotenoid levels were significantly lower than the concentrations in whole blood and plasma. Data presented from this study confirms that carotenoid status is disturbed in asthma. This study also demonstrated that while bioavailability is a significant issue when using carotenoids as supplements, improvements in plasma levels are reflected in the airways, where lutein has the most potential to achieve a biochemical and clinical effect.

Lysolecithins have been shown to have variety of biological actions, all centered around modification of cell permeability such effects include transfer of both cations and large molecules across cell membrane in cultured cell lines (Protective effects of D,L-camitine against arrhythmias induced by lyso phosphatidylcholine or reperfusion; Duan J, Moffat M P; Eur J Pharmacol. Jan. 17, 1991;192(3):355-63).

It was later concluded that lysophosphatidylcholine induces expression of adhesion molecules; however, the underlying molecular mechanisms of this are not well elucidated (Upregulation of endothelial adhesion molecules by lysophosphatidylcholine: Involvement of G protein-coupled receptor GPR4 ; Yani Zou; FEBS Journal, Volume 274, Number 10, March 2007, pp. 2573-2584(12)). In this study, the intracellular signaling by which lysophosphatidylcholine was shown to up-regulate vascular cell adhesion molecule-1and P-selectin was delineated using YPEN-1 and HEK293T cells. The results showed that lysophosphatidylcholine dose-dependently induced expression of vascular cell adhesion molecule-1 and P-selectin, accompanied by the activation of transcription factor nuclear factor κB. However, the nuclear factor κB inhibitor caffeic acid phenethyl ester (CAPE) and the antioxidant N-acetylcysteine only partially blocked lysophosphatidylcholine-induced adhesion molecules. Subsequently, it was found that the lysophosphatidylcholine receptor G protein-coupled receptor 4 (GPK4) was expressed in YPEN-1 cells and triggered the cAMP/protein kinase A/cAMP response element-binding protein pathway, resulting in upregulation of adhesion molecules. Further evidence showed that overexpression of human GPK4 enhanced lysophosphatidylcholine-induced expression of adhesion molecules in YPEN-1 cells, and enabled HEK293T cells to express adhesion molecules in response to lysophosphatidylcholine. In conclusion, the current study suggested two pathways by which lysophosphatidylcholine regulates the expression of adhesion molecules, the lysophosphatidylcholine/nuclear factor-κB/adhesion molecule and lysophosphatidylcholine/GPK4/cAMP/protein kinase A/cAMP response element-binding protein/adhesion molecule pathways, emphasizing the importance of the lysophosphatidylcholine receptor in regulating endothelial cell.

To attempt to enhance the bioavailability of carotenoids an in vitro study was conducted on Caco-2 Human Intestinal cells. The results suggest that pancreatic PLA2 and lysophosphatidylcholine are important in regulating the absorption of carotenoids in the digestive tract and support a simple diffusion mechanism for carotenoid absorption by intestinal epithelium (Lysophosphatidylcholine Enhances Carotenoid Uptake from Mixed Micelles by Caco-2 Human Intestinal Cells; Tatsuya Sugawara; 2001 American Society for Nutritional Sciences). Further, specifically to lutein, an in vivo single and repeat dose study was conducted in rats and demonstrated enhanced lutein bioavailability by lysophosphatidylcholine in plasma, liver and eyes (Enhanced lutein bioavailability by lyso-phosphatidylcholine in rats; R. Lakshminarayana; Molecular and Cellular Biochemistry 281: 103-110, 2006). It was also suggested that the luminal hydrolysis of phosphotidylcholine to lysophosphatidylcholine is necessary for intestinal uptake of lutein solubilized in mixed micelles.

Data presented from another study characterized the disposition of lutein after i.v. administration to rats and confirmed that lutein is preferentially distributed to the liver, spleen and lungs (Characterization of the Disposition of Lutein after i.v. Administration to Rats; Shirou Itagaki; Biol. Pharm. Bull. 29(10) 2123-2125 (2006)). Intravenous administration of lutein may provide effective antioxidant activities in these tissues, not only the eyes.

Another study showed that antioxidants may protect against the loss of lung function over time (Serum Carotenoids, a -Tocopherol, and Lung Function among Dutch Elderly; Linda Grievink et al.; Am J Respir Crit Care Med 2000;161:790-795). Reduced pulmonary function is an important predictor of mortality in the general population, and antioxidant are positively influence pulmonary function and it was found that lutein having a positive effect on FEV1 and FVC.

The results of another study (Epidemiologic Evidence Linking Antioxidant Vitamins to Pulmonary Function and Airway Obstruction; Holger J. Schunemann; Epidemiol Rev Vol. 23, No. 2, 2001) provided valuable data for lutein clinical development potential in fixed dose combination with known role of omega-3 essential fatty acids in asthma patients for synergistic effects.

Omega-3 polyunsaturated essential fatty acids such as fish oils these compounds have an important role in the treatment of certain inflammatory disorders as they modulate the arachidonic cascade in the direction of the anti-inflammatory series 5-leukotrienes. As such it was anticipated that fish oils would have an important role to play in the treatment of asthma. For unclear reasons, however the literature, including a Cochrane Collaboration analysis on this question, does not demonstrate any significant improvement in asthma patients using the omega-3 essential fatty acids.

Open clinical trials were conducted to test the effects of the combination of lysophospholipids, omega-3 fatty acids and carotenoids in the treatment of inflammation associated with intrinsic asthma and rheumatoid arthritis, at SSG Hospital, Vadodara. Patients who had met the inclusion criteria were enrolled in the trial after obtaining their written consent. Twenty patients suffering from intrinsic asthma and 20 patients suffering from rheumatoid arthritis were enrolled in the two trials respectively. The patients were administered one capsule twice a day for a period of two months. The complete blood hematology, X-ray, spirometry, and feedback were obtained at the start and at the end of two months, for the patients suffering from asthma. The complete hematology, X-ray, visual analogue scale and feedback recording was carried out at the start and at the end of two months for the patients suffering from rheumatoid arthritis. Results showed that there was an improvement in the FEV1% (>80%), erythrocyte sedimentation rate (ESR) (>50%) and C-reactive protein (CRP) (>70%) levels in the patients suffering from chronic intrinsic asthma. This was suggestive of an improvement in the lung function and an alleviation of the inflammatory condition. Also, the X-ray suggested an improvement in the chest congestion of 8 out of 20 patients enrolled in the asthma trial. The intake of concomitant drugs was also brought down in the case of patients suffering from asthma. In case of the patients suffering from arthritis, there was no measurable change in the hematology or CRP levels. However, from the visual analogue scales rated by the patients, it was found that in several patients, the incidence of nocturnal awakening, morning stiffness, and other motor activities had improved. However, as this response was not quantifiable, the effect could not be reported clearly. In both the clinical conditions, the patients expressed their willingness to continue with the medication as they had reported to feel better while under medication. Further trials will have to be conducted in a larger population so as to eliminate the placebo effects by running a double blind placebo controlled (DBPC) trial.

### Materials and Methods

### Materials

Carotenoids were obtained in the form of FloraGLO® brand lutein from Kemin Agrifoods North America. Omega-3 fatty acids were obtained as Marinol® C-38 from Loder's Croklaan. Lysophosphatidylcholine (LPC) including other lysophospholipids was obtained in the form of Lysoprin™ from Kemin Agrifoods Europe. Monopropylene glycol, food grade, was obtained from Manali Petrochemicals.

### Preparation of the Test Dose

The dose of administration was arrived at based on the standard usage of commercial grade of EPA and lysophospholipids and the dose of lutein was the same as in FloraGLO® brand lutein Kemin Industries (Table 20).Several test formulations were prepared in lab and checked for the consistency of the composition. Accelerated stability studies were conducted using the Rancimat and the shelf life of the composition was determined. The composition was prepared manually and encapsulated at the premises of an external manufacturer.

TABLE 20
Composition per capsule: (Soft gel: size 250 mg: fill volume: 230 mg)

| S. No. | Ingredients | Inclusion rate per capsule |
|---|---|---|
| 1 | Lysoprin ™ | 100 mg |
| 2 | Marinol ® C-38 | 100 mg |
| 3 | FloraGLO ® | 10 mg |
| 4 | Monopropylene Glycol | 20 mg |

### Experimental Trial Protocol

The trials were conducted under Good Clinical Practices compliance (8). The trials were carried out as per the described protocol at the Department of Medicine and Department of Orthopedics, The Government Medical College and S G Hospital, Vadodara after obtaining the approval from the ethics committee of this government authorized trial center. Enrolment of volunteers was done after ensuring that he/she met the inclusion/exclusion criteria for the trials (Table 21). A written informed consent of the volunteer was obtained prior to his enrolment in the trial. All the laboratory parameters were analyzed at the laboratory at SSG Hospital as per their authentic protocols. The visual analogue scale used for the quantification of pain, is a 10 point scale of 100 mm with markings for the patient to rate his perception of pain and other symptoms (7). The X-ray analysis was done at Urmi Labs, Vadodara. The data from the laboratory parameters analyzed and the vital signs was duly filled in by the investigators specific to each patient enrolled. The statistical analysis was carried out using the SAS package version 8.2.

TABLE 21
Trial overview

| S. No | | Asthma | Rheumatoid arthritis |
|---|---|---|---|
| 1 | Inclusion | Age (15-65)/male/female: persistent | Age (15-65)/male/female: |
| | criteria | chronic asthma for at least 1 year and | suffering from RA for at least 1 |
| | | FEV1 between 50-85% of predicted | year: meeting the RA criteria of |
| | | value with an increase of at least 15% in | the American college of |
| | | absolute FEV1 20-30 mins after | Rheumatology: arthritis |
| | | inhalation of short acting bronchodilator | patients of hand joints |
| | | on two clinical visits before initiation of | |
| | | active treatment | |
| 2 | Exclusion | Hospitalization with/without steroids | Hospitalization with/without |
| | criteria | within one month before enrolment: | steroids within one month |
| | | pregnant women, patients with renal or | before enrolment: pregnant |
| | | hepatic dysfunction: patients with serious | women, patients with renal or |
| | | or unstable cardiac dysfunction | hepatic dysfunction: patients |
| | | | with serious or unstable cardiac |
| | | | dysfunction |
| 3 | Duration of | 2 months | 2 months |
| | trial | | |
| 4 | No. Of | 20 | 20 |
| | patients | | |
| 5 | Parameters | Hemoglobin count, Hematocrit, Total | Hemoglobin count, Hematocrit, |
| | studied | cell count, Differential cell count, ESR, | Total cell count, Differential |
| | | CRP, | cell count, CRP, X-Ray, RA |
| | | X-Ray, Spirometry, concomitant | factor, concomitant medication, |
| | | medication. | Visual Analogue scale |
| 6 | Test | One capsule twice a day for two months | One capsule twice a day for |
| | medication | | two months |
| 7 | Follow up | One visit in between start and end of trial | One visit in between start and |
| | visits | | end of trial |

### Results and Discussion

Results of the Trial Done in Patients with Intrinsic Asthma

Table 22 depicts the average values obtained in the spirometry test done at the start and at the end of the two-month trial, in all the twenty patients. The values indicated a significant difference in the FEV₁ (forced expiratory volume in one second) and FEV₁% (the ratio of FEV₁ to forced vital capacity (FCV)) after the supplementation of the study medication for 2 months in the subjects. There was an improvement in the FEV values in 85% of the patients under study medication. The spirometry test was done to check the lung function. Improvement in FEV values were indicative of a positive response towards the study medication (Enright, et al. 1994; Gulledge).

TABLE 22
Paired t-test of pulmonary function test (pft)

| | PFT | Mean ± SD | | | |
|---|---|---|---|---|---|
| S. No | Parameters | PRE | POST | t-value | p-value |
| 1. | FVC | 1.458 ± 1.063 | 1.338 ± 0.750 | 0.47 | 0.645 |
| 2. | FEV_{I} | 0.797 ± 0.287 | 1.071 ± 0.591 | −2.53 | 0.021* |
| 3. | FEV_{I} % | 70.67 ± 17.74 | 87.80 ± 14.10 | −3.84 | 0.001* |
| 4. | PEF^{a} | 1.36 ± 0.629 | 2.04 ± 1.624 | −1.79 | 0.089 |
| 5. | VC^{b} | 2.13 ± 1.578 | 2.07 ± 1.489 | 0.09 | 0.935 |
| 6. | MVV^{c} | 44.64 ± 74.73 | 51.39 ± 74.37 | −0.27 | 0.792 |

*Indicates significant difference at p ≦ 0.05
^{a}peak Expiratory Flow
^{b}Vital Capacity
^{c}Maximum Voluntary Ventilation

Table 23 depicts the results of hematology carried out in outside laboratory. It could be observed that there was no significant difference in the values, pre and post trial in the subjects.

TABLE 23
Paired t-test of lab investigations of pre and post trial data of 20 subjects

| | | Mean ± SD | | | p- |
|---|---|---|---|---|---|
| Sr No | Lab parameters | PRE | POST | t-value | value |
| 1. | HB | 12.79 ± 1.154 | 12.73 ± 0.932 | 0.55 | 0.588 |
| 2. | Hematocrit | 41.20 ± 2.587 | 39.85 ± 2.346 | 3.94 | 0.001* |
| 3. | ESR | 18.05 ± 3.953 | 18.65 ± 3.815 | −0.95 | 0.353 |
| 4. | Tc | 7710 ± 1415.29 | 7485 ± 1004.87 | 1.08 | 0.292 |
| 5. | Dc P | 68.30 ± 7.794 | 66.50 ± 3.380 | 1.37 | 0.187 |
| 6. | L | 27.60 ± 6.435 | 30.85 ± 3.345 | −3.36 | 0.003* |
| | E | 4.85 ± 4.511 | 2.65 ± 2.477 | 2.40 | 0.027* |
| | B | 0.10 ± 0.447 | 0.00 ± 0.000 | 1.00 | 0.330 |
| | M | 0.15 ± 0.366 | 0.00 ± 0.000 | 1.83 | 0.083 |
| 7. | CRP | 10.95 ± 3.069 | 9.95 ± 3.187 | 1.67 | 0.111 |

Indicates significant difference at p ≦ 0.05

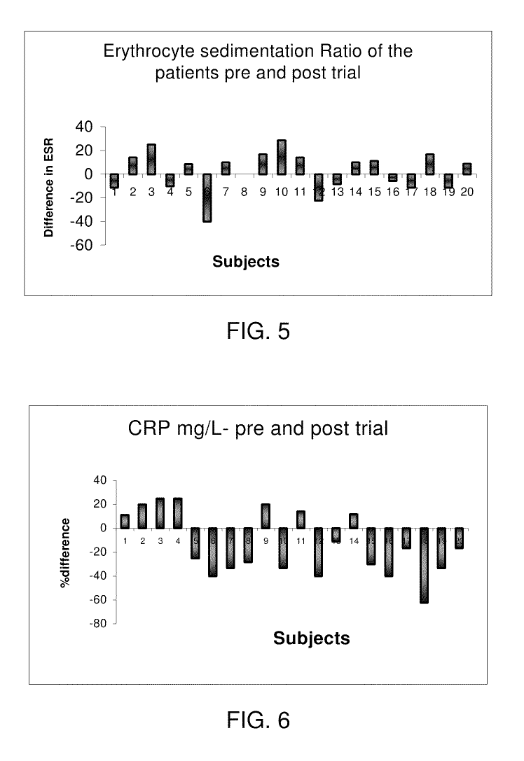

Although there were no significant changes in the blood counts, it was observed that the Erythrocyte Sedimentation Rate (ESR) was found to be lowered in almost 50% of the cases under study medication (FIG. 5). A decrease in the ESR in patients with inflammatory conditions is indicative of an improvement in the blood consistency which can be correlated with an improvement in the inflammatory condition (Reichmuth 2002).

Another marker of inflammatory conditions is the CRP (C-Reactive protein). CRP is an acute phase protein, which is found to be elevated under inflammatory conditions. The CRP values recorded pre and post trial in all the twenty patients. From the values obtained, it was observed that the CRP levels had been brought down in almost 70% of the cases enrolled in the trial (FIG. 6). This was indicative of alleviation in the inflammatory stress.

Table 24 shows the results of the X-ray interpretations of the 20 patients taken pre and post trial. It could be observed from the findings that during the 8 weeks of the trial period, over 50% of the patients showed an improvement in the lung congestion. It could be observed that at the start of the trial, 15 patients had bilateral congestion spread all over the chest. However, at the end of 8 weeks, there was an improvement in the chest congestion in over 9 cases. This was noted from the X-ray reports obtained pre and post trial.

TABLE 24
Findings from the X-ray analysis pre and post trial in the 20 patients

| | | Treatment t | | | | | |
|---|---|---|---|---|---|---|---|
| | | At time of | | After | | After | |
| S. | | Enrolment | | 4 week | | 8 week | |
| No | Clinical findings | No. | % | No. | % | No. | % |
| a. | Bilateral | 5 | 25.0 | 6 | 30.0 | 7 | 35.0 |
| b. | Bilateral all over chest | 15 | 75.0 | 11 | 55.0 | 6 | 30.0 |
| c. | Clear chest | — | — | 1 | 5.0 | 3 | 15.0 |
| d. | Bilateral few | — | — | 1 | 5.0 | 1 | 5.0 |
| e. | Bilateral scattered | — | — | 1 | 5.0 | 3 | 15.0 |

The overall tolerability and acceptability of the patients was recorded during the follow up visits. The study medication was found to be well tolerated and very well accepted by all the 20 patients. No adverse event was reported during the trial period.

The concomitant medication taken by the patients during the trial period was also recorded. Table 25 shows the number of doses of other drugs consumed on an average per patient at the start of the trial versus that consumed by the patients at the end of the trial. The data was subjected to Paired-t test analysis. It could be observed that there was a significant reduction in the number of doses of other drugs or concomitant medication consumed by the patients by the end of the trial. The significant reduction was observed after 8 weeks of medication, and the effect was found to sustain over the subsequent 4 weeks of the trial period.

TABLE 25
Paired t-test application to the data on the severity,
frequency of asthma attacks and the
doses of concomitant medicines used pre and post trial periods.

| | | At | After | After |
|---|---|---|---|---|
| S No | Parameters | Enrolment | 4 weeks | 8 weeks |
| 1 | Severity of attacks | 2.60 ± 1.14 | 2.30 ± 1.03 | 2.05 ± 1.19 |
| | based on markings in | | | |
| | scale | | | |
| 2 | Frequency of attacks | 2.65 ± 0.67 | 2.00 ± 0.85 | 1.75 ± 0.63 |
| 3 | Medication (number | 7.65 ± 2.05 | 5.70 ± 2.73 | 5.40 ± 1.84 |
| | of doses per week) | | | |

It was also noted that there were no drug—drug interactions during the trial period indicating the safety of the study medication when used even as an adjuvant along with any Beta agonist, or steroids, which were the most used concomitant drugs like deriphyllin, asthalin, salbutamol. The other symptoms that were recorded through verbal interactions and using rating scales were incidences of nocturnal awakening, ability to perform routine activities, and frequency of episodes of asthma attacks during the trial period. From the feedback obtained, it was found that there was an improvement in the ability to perform routine activity and also a reduction in the episodes of nocturnal awakening.

Results of the Trial Conducted in Patients Suffering from Rheumatoid Arthritis

Twenty patients suffering from rheumatoid arthritis were enrolled in the trial. The results of the hematology showed no difference as compared to the pre and post trial status (Table 26). The improvement in the clinical conditions associated with arthritis in the patients could be correlated only through the Visual Analogue scales, where the patients rated the pain intensity on a 10 point scale and correlated it to their ability to perform various activities with ease. The results of the scores obtained are as given in Table 27.

TABLE 26
Results of the laboratory investigations on patients with RA

| | Mean | | SD | | | |
|---|---|---|---|---|---|---|
| Parameters | Pre | Post | Pre | Post | t-value | p-value |
| Hemoglobin | 12.69 | 12.70 | 1.156 | 1.172 | −0.21 | 0.837 |
| Hematocrit | 40.15 | 40.40 | 0.933 | 0.681 | −1.31 | 0.204 |
| ESR | 33.10 | 31.40 | 6.464 | 5.295 | 3.13 | 0.006* |
| Total Count | 6205.0 | 6205.0 | 1012.28 | 945.613 | 0.00 | 1.000 |
| Platelets | 65.60 | 66.30 | 4.489 | 3.614 | −0.80 | 0.433 |
| Leucocytes | 31.05 | 31.6 | 3.818 | 3.966 | −0.57 | 0.576 |
| Eosinophils | 2.000 | 1.600 | 1.257 | 0.995 | 1.16 | 0.258 |
| Basophils | 0.000 | 0.000 | 0.000 | 0.000 | no variance | |
| Mast cells | 1.350 | 0.500 | 1.309 | 0.827 | 2.38 | 0.028* |

Indicates significant difference at p < 0.005

TABLE 27
Visual analogue scale scores

| | Mean | | SD | | | |
|---|---|---|---|---|---|---|
| OVERALL PAIN | Pre | Post | Pre | Post | t-value | p-value |
| OP | 28.00 | 26.00 | 7.327 | 7.182 | 2.99 | 0.008* |

SCALE QUESTIONS

| | | | | | | |
|---|---|---|---|---|---|---|
| 1. | 3.000 | 3.200 | 0.795 | 1.152 | −1.45 | 0.163 |
| 2. | 8.250 | 8.250 | 0.910 | 1.020 | 0.00 | 1.000 |
| 3. | 8.600 | 8.500 | 0.681 | 0.761 | 1.00 | 0.330 |
| 4. | 7.950 | 7.800 | 0.999 | 1.196 | 1.83 | 0.083 |
| 5. | 7.000 | 6.550 | 0.858 | 0.887 | 2.93 | 0.009* |
| 6. | 6.750 | 6.500 | 1.070 | 1.235 | 2.03 | 0.056* |
| Overall points | 41.55 | 40.80 | 3.103 | 3.665 | 2.07 | 0.052* |

Indicates significant difference at p < 0.005

The overall tolerability and acceptability by the patients was reported to be good and excellent by over 80% of the patients. The investigators had also rated it similarly. From the report, it appears that there is a reduction in the overall pain. From the trial data for individual patients there has been found to be a significant difference between the pre and post trial values marked on the visual analogue scales to perceive pain. The other benefits reported by the patients included freshness in the morning, reduction in pain complaints, slight increase in appetite and decrease in fatigue.

From the results obtained there appears to be an improvement in the clinical symptoms associated with both asthma and arthritis. However, the results are purely indicative and not conclusive in demonstrating the anti-inflammatory effects of the drug under study. The improvement in motor activity and reduction in the joint stiffness as reported by the patients suffering from arthritis is suggestive of the beneficial effects of the drug in alleviating inflammation at the synovial joints. The improvement in the chest congestion as recorded in the patients suffering from asthma and the improvement in the FEV values is a good indication of the beneficial effects of the drug for its anti-inflammatory effects. It could be that as a proposed mode of action, the inhibition of free arachidonic acid synthesis could further reduce the production of the leukotrienes that are synthesized from arachidonic acid. The mode of action of the drug has to be validated through animal experiments.

### CONCLUSION

From the open trial conducted to study the effect of the combination of lysophospholipids, more particular lysophosphatidylcholine (LPC), carotenoids and omega three fatty acids, in alleviating the clinical symptoms associated with intrinsic asthma and rheumatoid arthritis, it was reported that the combination indeed had some beneficial effects. There has been some improvement in the lung function of the patients suffering from asthma and also a decrease in the CRP levels has been recorded. However, in the patients suffering from rheumatoid arthritis, no such clinical parameter was definitive of the effect although the visual analogue scoring reported improvement.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

### REFERENCES

1. Jesus Balsinde, Maria A. Balboa, Paul A. Insel, and Edward A. Dennis, Regulation and Inhibition of Phospholipase A2 Annu. Rev. Pharmacol. Toxicol. 1999. 39:175-89
2. Li Zhou and Ake Nilsson, Sources of eicosanoid precursor fatty acid pools in tissues J. Lipid Res. Volume 42, 2001, 1521-1542
3. Enright P L, Lbowitz M D, Cockroft D W, Physiologic measures: pulmonary function tests. Asthma outcome, Am J Respir Crit Care Med, February 1994; 149(2 Pt 2): S9-18;
4. Daniel Reichmuth, M D, Uses of Erythrocyte Sedimentation Rate in Allergy/Immunology, Pediatric Asthma, Allergy & Immunology, June 2002, Vol. 15, No. 3: 167-171
5. Jo Gulledge Asthma Management: Clinical Pathways, Guidelines, and Patient Education
6. Bonnie Bruce and James F Fries The Stanford Health Assessment Questionnaire: Dimensions and Practical Applications, Health Qual Life Outcomes. 2003; 1: 20.
7. Good Clinical Practice in FDA-Regulated Clinical Trials, U.S Food and Drug Administration.

We claim:

1. A method of treating or ameliorating the effects of inflammation, comprising the step of administering a therapeutically effective amount of a lysophospholipid.

2. The method of claim 1, further comprising the co-administration of a phospolipid which, in combination, provide a therapeutically effective amount of the compounds.

3. The method of claim 1, further comprising the co-administration of a compound selected from the group consisting of phospholipids, omega-3 fatty acids, prebiotics, probiotics, and lutein, which, in combination, provide a therapeutically effective amount of the compounds.

4. The method of claim 1, further comprising the co-administration of an anti-inflammatory compound which, in combination, provide a therapeutically effective amount of the compounds.

5. The method of claim 1, wherein the effects of inflammation are selected from the group consisting of juvenile diabetes, atherosclerosis, hypothyroidism, rheumatoid arthritis, psoriasis, asthma, colitis, multiple sclerosis, and systemic lupus erythematosus.

6. The method of claim 1, wherein the administration is selected from the group consisting of oral, parenteral, intravenous, topical, nasal, inhaled, ocular, subcutaneous and intrarectal.

7. The method of claim 1, wherein the lysophospholipid is lysophosphatidylcholine.

8. A method of preventing the effects of inflammation, comprising the step of administering a therapeutically effective amount of a lysophospholipid.

9. The method of claim 8, further comprising the co-administration of a phospolipid which, in combination, provide a therapeutically effective amount of the compounds.

10. The method of claim 8, further comprising the co-administration of a compound selected from the group consisting of phospholipids, omega-3 fatty acids, prebiotics, probiotics, and lutein, which, in combination, provide a therapeutically effective amount of the compounds.

11. The method of claim 8, further comprising the co-administration of an anti-inflammatory compound which, in combination, provide a therapeutically effective amount of the compounds.

12. The method of claim 8, wherein the effects of inflammation are selected from the group consisting of juvenile diabetes, atherosclerosis, hypothyroidism, rheumatoid arthritis, psoriasis, asthma, colitis, multiple sclerosis, and systemic lupus erythematosus.

13. The method of claim 8, wherein the administration is selected from the group consisting of oral, parenteral, intravenous, topical, nasal, inhaled, ocular, subcutaneous and intrarectal.

14. The method of claim 8, wherein the lysophospholipid is lysophosphatidylcholine.

15. A composition for treating the effects of inflammation, comprising a therapeutically effective amount of a lysophospholipid.

16. The composition of claim 15, further comprising a phospolipid which, in combination, provide a therapeutically effective amount of the compounds.

17. The composition of claim 15, further comprising the co-administration of a compound selected from the group consisting of phospholipids, omega-3 fatty acids, prebiotics, probiotics, and lutein, which, in combination, provide a therapeutically effective amount of the compounds.

18. The composition of claim 15, further comprising an anti-inflammatory compound which, in combination, provide a therapeutically effective amount of the compounds.

19. The composition of claim 15, wherein the effects of inflammation are selected from the group consisting of juvenile diabetes, atherosclerosis, hypothyroidism, rheumatoid arthritis, psoriasis, asthma, colitis, multiple sclerosis, and systemic lupus erythematosus.

20. The composition of claim 15, wherein the lysophospholipid is lysophosphatidylcholine
